(19) United States
(12) Patent Application Publication
(10) Pub. No.: US 2022/0332786 A1
(43) Pub. Date: Oct. 20, 2022

(54) CHIMERIC ANTIGEN RECEPTOR AND APPLICATION THEREOF

(71) Applicant: BEIJING ZHUTIAN TECHNOLOGY DEVELOPMENT CO., LTD, Beijing (CN)

(72) Inventor: Xiaodong LI, Beijing (CN)

(73) Assignee: BEIJING ZHUTIAN TECHNOLOGY DEVELOPMENT CO., LTD, Beijing (CN)

(21) Appl. No.: 17/628,577

(22) PCT Filed: Sep. 22, 2020
(86) PCT No.: PCT/CN2020/116721

§ 371 Date: Jul. 3, 2022

(30) Foreign Application Priority Data
Jul. 22, 2019 (CN) 2019 10659680.0

(51) Int. Cl. C07K 14/725; C07K 14/705; C07K 16/28; A61K 38/21; A61K 38/20; A61K 38/17; A61K 39/395

(52) U.S. Cl. CPC C07K 14/7051; C07K 14/70521; C07K 16/2818; C07K 16/2827; A61K 38/217; A61K 38/20; A61K 38/1774; A61K 39/3955; C07K 2319/02; C07K 2319/03

(57) ABSTRACT

A chimeric antigen receptor includes a) an extracellular target molecule combination domain, used for a specific-binding target molecule; b) an intracellular signaling domain including at least one intracellular activation signaling domain and/or at least one intracellular detection signaling domain; and c) a transmembrane domain, used to connect the extracellular target molecule combination domain and the intracellular signaling domain, and fix the two domains on a cell membrane. Activation of the intracellular signaling domain at least relies on combination of the extracellular target molecule combination domain with the target molecule, and the intracellular activation signaling domain contains a molecule or a fragment having a catalytic functional group. The present chimeric antigen receptor combines various means to create and apply an artificial molecular machine, thus having the strengths of an immune checkpoint inhibitor and of cell therapy at the same time, and providing a solution for improving treatment of solid tumors.

### CROSS REFERENCE TO THE RELATED APPLICATIONS

This application is the national phase entry of International Application No. PCT/CN2020/116721, filed on Sep. 22, 2020, which is based upon and claims priority to Chinese Patent Application No. 201910659680.0, filed on Jul. 22, 2019, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBYZL003-PKG_Sequence Listing.txt, created on 04/17/2022, and is 62,258 bytes in size.

### TECHNICAL FIELD

The present application relates to chimeric antigen receptor and application thereof, belonging to the field of biomedicine.

### BACKGROUND

Cancer is one of the most important public health burdens in the world, accounting for about 1 in every 6 deaths in the world, and most cancer types, especially malignant tumors with low survival rates, frequently occur in the elderly population (World Health Organization, WHO report on cancer, 2020). For example, in the United States, cancer was the second leading cause of death in 2019 after heart disease, with more than a hundred types of cancer due to many different causes and up to 80% of people over 55 years of age diagnosed with cancer (Siegel R L et al., CA: a cancer journal for clinicians. 2020 January; 70(1):7-30.). In China, with more public health problems arising from the aging of the Chinese population, the acceleration of industrial urbanization, and the widespread prevalence of unhealthy lifestyles, the Chinese government's cancer prevention and control situation is unprecedentedly severe. Statistics from the recent cancer report GLOBOCAN 2018 show that the number of new cancer cases and cancer deaths in China account for 23.7% and 30% of the world's respectively, and the standardized incidence and standardized mortality ranks 68th and 12th in the world, respectively. Cancer has become one of main diseases threatening the public health of Chinese residents (Cao Maomao et al., Chinese Journal of Clinical Oncology, 2019, 46, 145-149; Bray F et al., CA: a cancer journal for clinicians. 2018 November; 68(6):394-424.). It can be seen that how to overcome the low survival rate of cancer has become the focus of cancer prevention and control in human society at this stage.

One of the reasons why cancer is very deadly is that cancer cells grow and divide very rapidly, and become malignant tumors at the late stage of cancer. If the malignant proliferation of cancer cells is not effectively controlled, it will lead to the death of patients. In addition, cancer cells can break the boundaries of normal tissues through the blood circulatory system or the lymphatic system to invade adjacent tissues and even spread to various parts of the body, which is a process known as cancer metastasis and spread and further reduces the ability to treat and eradicate cancer cells (Gupta G P et al. Cell. 2006 Nov. 17; 127(4):679-95.). In addition, solid tumors differ from hematological cancers in that they have complex dynamic ecosystems, tumor microenvironments, and high tumor heterogeneity, involving interactions among many kinds of cells, such as active interactions between cancer cells and stromal cells. Cancer cells can affect the development of tumors and the response of tumors to therapy by regulating the body's adaptive immune response and innate immune response, which further increases the difficulty of treating solid tumors.

While the immune system constantly protects body from various diseases, there are times when it doesn't provide the protection our bodies need, such as when the immune system is fighting cancer. One of the main reasons currently known is that tumors can evade the surveillance of the immune system in order to improve their survival rate, i.e., tumor immune evasion, which suppresses related functions of the immune system including such as adaptive immune system and innate immune system, by interfering with the anticancer immune response of human immune cells. From the perspective of basic immunology research, people are beginning to discover the important role of T lymphocytes, one of the main components of the adaptive immune system, in the adaptive immune response and how the function and behavior of T cells are systematically controlled. T cell receptor signaling mediated by co-stimulatory and co-inhibitory receptors controls T cell fate decisions such as activation, proliferation, differentiation, potency, and survival (Smith-Garvin J E et al., Annual review of immunology. 2009 Apr. 23; 27:591-619.). However, solid tumors precisely use certain immune checkpoint signaling pathways in their tumor microenvironment to inhibit and shut down the immune cell functions of the adaptive immune system against cancer cells, especially the T lymphocyte function, resulting in cancer cells producing resistance to immune recognition and killing, thereby making T lymphocytes no longer able to function normally (Pardoll D M, Nature Reviews Cancer. 2012 April; 12(4):252-64.; Pardoll D M, Nature immunology. 2012 December; 13(12):1129-32). For example, cancer cells express a large number of immune checkpoint-related signaling molecule ligands (such as programmed death receptor ligand 1, PD-L1) in the immunosuppressive tumor microenvironment, and by binding with the corresponding immune checkpoint receptor molecules (such as programmed death receptor 1, PD-1) expressed on the surface of T lymphocytes to achieve inhibitory functions against T lymphocytes, including inhibiting the secretion and release of pro-inflammatory or anti-tumor cytokines, limiting the proliferation and differentiation of T cells, inhibiting the function of effector T cells, promoting the dysfunction and exhaustion of T cells and even death by apoptosis, so as to realize the limitation of the functions of T lymphocytes that recognize cancer cells to inhibit cancer cells and kill cancer cells.

In addition, the innate immune system also plays an extremely important role in the solid tumor microenvironment. For example, inflammatory cells are an important part of the tumor ecosystem, and a type of phagocyte called tumor-associated macrophage represents one of the most abundant stromal components in the tumor microenvironment and is therefore highly evident stromal target cells in many solid tumors. Tumor-associated macrophages are the most numerous leukocytes in some solid tumors, and in most human tumor types, infiltration of tumor-associated macrophages or enrichment of tumor-associated macrophage-associated gene signatures or phenotypes is highly correlated with poor prognosis and disease outcomes. A large number of tumor-associated macrophages accumulate in the tumor microenvironment and are often closely associated with specific pathological features after cancer development, such as immunosuppression, neovascularization, infiltration, metastasis, and adverse response to therapy, which strongly implies that tumor-associated macrophages promote tumor growth. As the main branch of human immune defense, the innate immune system acts as the first line of non-specific defense against foreign bodies, microbial infections, dying cells, dead cells, diseased cells, malignant cell metastasis and the like. Professional phagocytes in the innate immune system include many different types of white blood cells, such as neutrophils, macrophages, monocytes, mast cells, dendritic cells, and B cells, and play an important role during the immune response involved in fighting infection and maintaining tissue health. Macrophages are not only one of the constituent cells of the innate immune system, but also belong to professional antigen-presenting cells, which rely on cell surface molecules such as germline-encoded pattern recognition receptors to rapidly identify and respond to foreign bodies, dying cells, dead cells, pathological cells, structural components of microorganisms, or cancer cell-related molecules and the like, to coordinate downstream inflammatory responses or anti-tumor responses. Professional antigen-presenting cells include macrophages, B cells, Langerhans cells, and dendritic cells. The process of cross-presentation between innate and adaptive immune cells is also critical for activating the adaptive immune system. For example, antigen-presenting cells mediate the processing and cross-presentation of antigens to naive T cells, leading to T cell activation. The key to the interaction between the innate and adaptive immune systems is the ability of antigen-presenting cells to phagocytose target microorganisms or target cells (such as bacteria, infected cells, or cancer cells) through phagocytosis. Phagocytosis is a multi-step cell process, involving steps such as recognition, phagocytosis of cells and lysosomal digestion of target microorganisms or target cells, which are regulated by receptor-ligand interactions between the target microorganisms or target cells and phagocytes. The phagocytosis of the body is divided into two categories: one phagocytosis refers to resisting foreign bodies or microorganisms to remove and degrade foreign bodies or microorganisms that cause disease, inducing pro-inflammatory signaling through cytokine secretion, and recruiting immune phagocytes to produce effective inflammatory response; the other phagocytosis refers to the followings: phagocytes perform specific clearance of apoptotic cells, dead cells, diseased cells, malignant cells and even tumor cells, which does not cause damage to surrounding tissues or induce pro-inflammatory immune responses. Though healthy tissue cells express anti-phagocytosis molecules to avoid being subjected to phagocytosis and clearance by phagocytes, cancer cells cunningly rely on similar mechanisms to evade immune system-mediated recognition, killing, and clearance. One of the important mechanisms is that cancer cells rely on the expression of “don't eat me” signaling molecules, including molecules such as PD-L1, to strengthen phagocyte-related inhibitory signals and thus inhibit phagocytic phagocytosis and clearance; the above signal molecules bind to receptor molecules such as the immune checkpoint PD-1 on the surface of phagocytes to inhibit the phagocytosis and clearance of cancer cells by phagocytes, thereby achieving immune escape.

Therefore, people are increasingly aware of the importance of making better use of the power of the immune system to fight against disease. In recent years, with the deepening comprehensions and understanding of cancer and the immune system in modern science, cancer immunotherapy as one of the weather vanes leading the global biopharmaceutical industry, has developed rapidly, and has made breakthroughs, opening up a new way for the next generation of cancer immunotherapy. Cancer immunotherapy, by which a patient's own immune system regains the ability to fight against cancer, is similar to the way the immune system fights disease-causing viruses or bacteria. Such treatment modulates the ability of the patient's own immune system and increases the durability of the therapy. Of course, different cancer immunotherapies work on a patient's immune system by different ways. For example, some therapies promote and enhance the immune response against cancer, while others allow the immune system to better identify, target and kill cancer cells.

One of the most revolutionary cancer immunotherapies refers to immune checkpoint modulator, especially immune checkpoint inhibitor. The human immune system requires many checks and balances to protect itself from pathogens while avoiding attacks on its normal cells. To do this, the immune system employs proteins called “immune checkpoints” (such as PD-1) to suppress the immune response. Unexpectedly, years of research have shown that certain tumors express a large number of immune checkpoint-related signaling molecule ligands (such as PD-L1, which is the above-mentioned “don't eat me” signal) to suppress or even prevent the immune response, thereby preventing being attacked from the immune system. It is as if cancer cells have stepped on the brakes against the immune system to achieve the purpose of immune escape. Among the immune checkpoint inhibitors that have been discovered, the inhibitors targeting the immune checkpoint PD-1 and its ligand PD-L1 are the most representative and have therapeutic prospects. Such inhibitors can target the tumor molecular marker PD-L1 and its receptor PD-1, blocking the inhibition of immune cells by cancer cells, as if cancer cells releases the brakes against the immune system, allowing the immune system to re-identify and kill the corresponding tumors. In 2014, the FDA pre-approved the first tumor immune drug in history, Merck's PD-1 monoclonal antibody inhibitor Keytruda. Long-term data published in 2016 shows that Keytruda significantly improves survival time of patients with advanced melanoma: 40% of treated patients (655 in total) survives for more than 3 years, in stark contrast to pre-immunotherapy treatments that allow patients to survive only for several months. Former US President Jimmy Carter with 95 years old, is a long-term user of the drug. In May 2017, Keytruda received rapid approval from the FDA again, becoming the first FDA-approved anticancer drug based on tumor biomarkers without distinguishing tumor origin. It can be called a broad-spectrum anticancer drug for various types of solid tumors. At the end of 2018 and the first half of 2019, the domestic PD-1 antibody drugs of Chinese pharmaceutical companies TopAlliance Biosciences Inc., Innovent Biologics, Inc. and Hengrui Pharmaceuticals Co., Ltd. were also approved for marketing.

Another promising cancer immunotherapy is cell therapy, especially Chimeric Antigen Receptor (CAR) T cell therapy, namely CAR-T cell therapy, which uses genetic engineering and synthetic biology to modify cells to further realize the recognition and killing of specific cancer cells. The successful advent of such therapy is a milestone, representing a new change in terms of cancer treatment, significantly increasing the choice and grasp of human tumor treatment. In recent years, CAR-T cell therapy has achieved good results in the clinical treatment of blood cancers (including lymphoma and lymphocytic leukemia). Especially CAR-T cell therapy targeting CD19 is the most successful. At present, the composition of CAR molecules mainly includes: the extracellular antigen recognition domain from antigen-specific single-chain antibody fragment, the spacer domain between the antigen recognition domain and the transmembrane domain from molecular linker fragments such as IgG family proteins, transmembrane domain from molecular transmembrane fragment such as CD28 or CD8, the intracellular co-stimulatory signaling domain and the intracellular activation signaling domain. The CAR molecule based on the above design can enable the modified T cells to realize the function of identifying specific cancer cells and activating their intracellular T cell signaling independent of classical HLA. Novartis CAR-T cell therapy Kymriah, which was unanimously approved by the FDA in 2017, is the first gene therapy approved by the FDA in human history for the treatment of B-cell precursor acute lymphoblastic leukemia. Data published in 2017 shows that patients who receives the therapy could achieve an overall remission rate of up to 83%, which is unprecedented.

Though CAR-T and other cell therapies have achieved exciting results in the treatment of blood cancers, cell therapy faces many challenges in the treatment of solid tumors such as the complex immunosuppressive tumor microenvironment and high tumor heterogeneity of solid tumors, and thus still needs to be further explored and developed. In the solid tumor microenvironment, cancer cells rely on the expression of “don't eat me” signaling molecules such as PD-L1 to inhibit the function of killing cancer cells of T cells and phagocytes and thus achieve immune escape, which seriously hinders the progress of the cell therapy in terms of treatment of solid tumors. Therefore, based on the understanding of the multi-faceted functional roles of T lymphocytes and phagocytes in the occurrence and development of tumor tissues, it is very attractive treatment strategies to selectively strengthen and enhance the anti-tumor effects of T lymphocytes and phagocytes in the tumor microenvironment.

In addition, it is known that immunosuppressive signals are highly involved in infections, inflammatory diseases, immune diseases, neurological diseases and other diseases. Thus, the cell therapy based on modification of immunosuppressive signal in the present application, is also applicable to treat diseases such as infections, inflammatory diseases, immune diseases and neurological diseases.

The methods and compositions disclosed in the present application can enhance the body to clear the corresponding disease focus in the treatment of diseases such as various cancers, infections, inflammatory diseases, immune diseases, and nervous system diseases, and especially in terms of solid tumors, can kill and clear solid cancer cells to meet the above needs.

### SUMMARY

According to one aspect of the present application, there is provided a chimeric antigen receptor. Such technology combines various means such as tumor immunology, synthetic biology, and molecular cell engineering to create an artificial molecular machine for regulating immune cell function, thus having the strengths of an immune checkpoint inhibitor and of cell therapy at the same time, and providing a solution for improving treatment of solid tumors.

A chimeric antigen receptor comprising:
a) an extracellular target molecule binding domain for specifically binding to a target molecule;
b) an intracellular signaling domain comprising at least one intracellular activation signaling domain and/or at least one intracellular detection signaling domain; and
c) a transmembrane domain, used to connect the extracellular target molecule binding domain and the intracellular signaling domain, and anchor these two domains on a cell membrane;
wherein, an activation of the intracellular activation signaling domain at least relies on the binding of the extracellular target molecule binding domain to the target molecule; and the intracellular activation signaling domain contains a molecule or a fragment having a catalytic domain.

A chimeric antigen receptor comprising:
a) an extracellular target molecule binding domain for specifically binding to a target molecule;
b) an intracellular signaling domain comprising at least one intracellular activation signaling domain; and
c) a transmembrane domain, used to connect the extracellular target molecule binding domain and the intracellular signaling domain, and anchor these two domains on a cell membrane;
wherein, an activation of the intracellular activation signaling domain at least relies on the binding of the extracellular target molecule binding domain to the target molecule; and the intracellular activation signaling domain contains a molecule or a fragment having a catalytic domain.

A chimeric antigen receptor comprising:
a) an extracellular target molecule binding domain for specifically binding to a target molecule;
b) an intracellular signaling domain comprising at least one intracellular activation signaling domain and at least one intracellular detection signaling domain; and
c) a transmembrane domain, used to connect the extracellular target molecule binding domain and the intracellular signaling domain, and anchor these two domains on a cell membrane;
wherein, an activation of the intracellular activation signaling domain at least relies on the binding of the extracellular target molecule binding domain to the target molecule; and the intracellular activation signaling domain contains a molecule or a fragment having a catalytic domain.

A chimeric antigen receptor comprising:
a) an extracellular target molecule binding domain for specifically binding to a target molecule;
b) an intracellular signaling domain comprising at least one intracellular detection signaling domain; and
c) a transmembrane domain, used to connect the extracellular target molecule binding domain and the intracellular signaling domain, and anchoring these two domains on a cell membrane.

A chimeric antigen receptor comprising:
a) an extracellular target molecule binding domain for specifically binding to a target molecule;
b) an intracellular signaling domain comprising at least one intracellular detection signaling domain; and
c) a transmembrane domain, used to connect the extracellular target molecule binding domain and the intracellular signaling domain, and anchor these two domains on a cell membrane;
wherein, the intracellular detection signaling domain is at least one of CD3ζ ITAM1 fragment, CD3ζ ITAM2 fragment, CD3ζ ITAM3 fragment, FcRIIA ITAM fragment, FcRγ ITAM fragment, DAP12 ITAM fragment, and CD3ε ITAM fragment.

Optionally, the intracellular signaling domain further comprises at least one intracellular activation signaling domain.

Optionally, an activation of the intracellular activation signaling domain at least relies on the binding of the extracellular target molecule binding domain to the target molecule; and the intracellular activation signaling domain contains a molecule or a fragment having a catalytic domain.

Optionally, the intracellular activation signaling domain comprises at least one of receptor-type tyrosine kinase, receptor-type tyrosine kinase fragment, non-receptor-type tyrosine kinase and non-receptor-type tyrosine kinase fragment.

Optionally, the receptor-type tyrosine kinase is at least one of EGFR, HER2, HER3, HER4, InsR, IGF1R, IRR, PDGFRα, PDGFRβ, Kit, CSFR, FLT3, VEGFR-1, VEGFR-2, VEGFR-3, FGFR1, FGFR2, FGFR3, FGFR4, CCK4, trkA, trkB, trkC, ROR1, ROR2, MuSK, MET, Ron, Axl, Tyro3, Mer, TIE1, TIE2, EphA1, EphA2, EphA3, EphA4, EphA5, EphA6, EphA7, EphA8, EphA10, EphB1, EphB2, EphB3, EphB4, EphB6, Ret, RYK, DDR1, DDR2, ROS, Lmr1, Lmr2, Lmr3, LTK, ALK and STYK1, and the non-receptor tyrosine kinase is at least one of Abl, Arg, Tnk1, Ack, CSK, CTK, FAK, Pyk2, Fer, Fes, JAK1, JAK2, JAK3, Tyk2, Blk, Fgr, FRK, Fyn, Hck, Lck, Lyn, Brk, Src, Srm, Yes, Syk, ZAP70, Etk, Btk, ITK, TEC and TXK.

Optionally, the intracellular activation signaling domain comprises at least one of an amino acid sequence comprising SEQ ID NO:042, an amino acid sequence comprising SEQ ID NO:044, an amino acid sequence comprising SEQ ID NO:046, an amino acid sequence comprising SEQ ID NO:048, an amino acid sequence comprising SEQ ID NO: 050, and an amino acid sequence comprising SEQ ID NO: 052.

Optionally, the chimeric antigen receptor further comprises an intracellular detection signaling domain, and the intracellular detection signaling domain is connected to the intracellular activation signaling domain.

Optionally, the intracellular detection signaling domain comprises at least one immunoreceptor tyrosine-based activation motif ITAM.

Optionally, the intracellular detection signaling domain is at least one of CD3ζ ITAM1 fragment, CD3ζ ITAM2 fragment, CD3ζ ITAM3 fragment, FcRIIA ITAM fragment, FcRγ ITAM fragment, DAP12 ITAM fragment and CD3ε ITAM fragment.

Optionally, the intracellular detection signaling domain comprises at least one signaling domain of a molecule selected from 2B4, CD244, BTLA, CD3δ, CD3γ, CD3ε, CD3ζ, CD5, CD28, CD31, CD72, CD84, CD229, CD300a, CD300f, CEACAM-1, CEACAM-3, CEACAM-4, CEACAM-19, CEACAM-20, CLEC-1, CLEC-2, CRACC, CTLA-4, DAP10, DAP12, DCAR, DCIR, Dectin-1, DNAM-1, FcεRIα, FcεRIβ, FcγRIB, FcγRI, FcγRIIA, FcγRIIB, FcγRIIC, FcγRIIIA, FCRL1, FCRL2, FCRL3, FCRL4, FCRL5, FCRL6, G6b, KIR, KIR2DL1, KIR2DL2, KIR2DL3, KIR2DL4, KIR2DL5, KIR2DL5B, KIR2DS1, KIR2DS3, KIR2DS4, KIR2DS5, KIR3DL1, KIR3DL2, KIR3DL3, KIR3DS1, KLRG1, LAIR1, LILRB1, LILRB2, LILRB3, LILRB4, LILRB5, MICL, NKG2A, NKp44, NKp65, NKp80, NTB-A, PD-1, PDCD6, PILR-α, Siglec-2, Siglec-3, Siglec-5, Siglec-6, Siglec-7, Siglec-8, Siglec-9, Siglec-10, Siglec-11, Siglec-12, Siglec-14. Siglec-15, Siglec-16, SIRPα, SLAM, TIGIT, TREML1 and TREML2.

Optionally, the intracellular detection signaling domain comprises at least one of an amino acid sequence comprising SEQ ID NO:020, an amino acid sequence comprising SEQ ID NO:022, an amino acid sequence comprising SEQ ID NO:024, an amino acid sequence comprising SEQ ID NO:026, an amino acid sequence comprising SEQ ID NO:028, an amino acid sequence comprising SEQ ID NO:030, an amino acid sequence comprising SEQ ID NO:032, an amino acid sequence comprising SEQ ID NO:034, an amino acid sequence comprising SEQ ID NO:036, an amino acid sequence comprising SEQ ID NO:038, and an amino acid sequence comprising SEQ ID NO:040.

Optionally, the target molecule bound by the extracellular target molecule binding domain comprises at least one of the following molecules: immunosuppressive signal-related molecule, tumor surface antigen molecular marker, and specific antigen peptide-histocompatibility complex molecule on cell surface.

Optionally, the extracellular target molecule binding domain is at least one of molecules that can identify and bind target molecules such as immunosuppressive signal-related molecules or tumor surface antigen molecular markers, or can be common monoclonal antibody or single-chain variable fragment and antigen-recognition-binding fragment thereof in existing chimeric antigen receptor, anti-immunosuppressive signal-related molecule monoclonal antibody and the antigen-recognition-binding fragment thereof, or anti-tumor surface antigen molecular marker monoclonal antibody and the antigen-recognition-binding fragment thereof. Preferably, the extracellular target molecule binding domain is at least one of molecules that can identify and bind to immunosuppressive signal-related molecules or tumor surface antigen molecular markers.

Optionally, the extracellular target molecule binding domain comprises at least one of target molecule binding domain of a molecule selected from PD-1, Truncated PD-1, PD-1 variant, antibody against PD-L1, PD-L1-binding fragment, monoclonal antibody binding to PD-L1, polyclonal antibody binding to PD-L1, synthetic antibody binding to PD-L1, human antibody binding to PD-L1, humanized antibody binding to PD-L1, single-domain antibody binding to PD-L1, nanobody binding to PD-L1, single-chain variable fragment antibody binding to PD-L1 and the binding fragment antibody thereof.

Optionally, the extracellular target molecule binding domain comprises at least one of an amino acid sequence comprising SEQ ID NO:001, an amino acid sequence comprising SEQ ID NO:003, an amino acid sequence comprising SEQ ID NO:005, an amino acid sequence comprising SEQ ID NO:007, an amino acid sequence comprising SEQ ID NO:009 and an amino acid sequence comprising SEQ ID NO:011.

Optionally, the transmembrane domain is at least one transmembrane domain of the transmembrane protein selected from PD-1, PD-L1, PD-L2, 4-1BB, 4-1BBL, ICOS, GITR, GITRL, OX40, OX40L, CD40, CD40L, CD86, CD80, CD2, CD28, B7-DC, B7-H2, B7-H3, B7-H4, B7-H5, B7-H6, B7-H7, VSIG-3, VISTA, SIRPα, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, Siglec-15, Siglec-16, DAP10, DAP12, NKG2A, NKG2C, NKG2D, LIR1, KIR, KIR2DL1, KIR2DL2, KIR2DL3, KIR2DL4, KIR2DL5A, KIR2DL5B, KIR2DS1, KIR2DS3, KIR2DS4, KIR2DS5, KIR3DL1, KIR3DL2, KIR3DL3, KIR3DS1, KLRG1, KLRG2, LAIR1, LAIR2, LILRA1, LILRA2, LILRA3, LILRA4, LILRA5, LILRB1, LILRB2, LILRB3, LILRB4, LILRB5, 2B4, BTLA, CD160, LAG-3, CTLA-4, CD155, CD112, CD113, TIGIT, CD96, CD226, TIM-1, TIM-3, TIM-4, Galectin-9, CEACAM-1, CD8a, CD8b, CD4, MERTK, AXL, Tyro3, BAIL MRC1, MRC2, FcγR1, FcγR2A, FcγR2B1, FcγR2B2, FcγR2C, FcγR3A, FcγR3B, FcεR2, FcεR1, FcRn, Fcα/μR and FcαR1.

Optionally, the transmembrane domain comprises at least one of an amino acid sequence comprising SEQ ID NO:012 and an amino acid sequence comprising SEQ ID NO:014.

Optionally, the intracellular detection signaling domain is connected to the intracellular activation signaling domain, and the intracellular detection signaling domain is located between the transmembrane domain and the intracellular activation signaling domain.

Optionally, an extracellular spacer domain is further included between the extracellular target molecule binding domain and the transmembrane domain.

Optionally, the extracellular spacer domain comprises at least one of an amino acid sequence comprising SEQ ID NO:016 and an amino acid sequence comprising SEQ ID NO:018.

Optionally, the chimeric antigen receptor further comprises an intracellular spacer domain, and the intracellular spacer domain is located between the transmembrane domain and the intracellular signaling domain and connects these two domains together.

Optionally, the intracellular spacer domain is an extension of the transmembrane domain, comprising at least one molecule selected from PD-1, PD-L1, PD-L2, 4-1BB, 4-1BBL, ICOS, GITR, GITRL, OX40, OX40L, CD40, CD40L, CD86, CD80, CD2, CD28, B7-DC, B7-H2, B7-H3, B7-H4, B7-H5, B7-H6, B7-H7, VSIG-3, VISTA, SIRPα, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, Siglec-15, Siglec-16, DAP10, DAP12, NKG2A, NKG2C, NKG2D, LIR1, KIR, KIR2DL1, KIR2DL2, KIR2DL3, KIR2DL4, KIR2DL5A, KIR2DL5B, KIR2DS1, KIR2DS3, KIR2DS4, KIR2DS5, KIR3DL1, KIR3DL2, KIR3DL3, KIR3DS1, KLRG1, KLRG2, LAIR1, LAIR2, LILRA1, LILRA2, LILRA3, LILRA4, LILRA5, LILRB1, LILRB2, LILRB3, LILRB4, LILRB5, 2B4, BTLA, CD160, LAG-3, CTLA-4, CD155, CD112, CD113, TIGIT, CD96, CD226, TIM-1, TIM-3, TIM-4, Galectin-9, CEACAM-1, CD8a, CD8b, CD4, MERTK, AXL, Tyro3, BAI1, MRC1, MRC2, FcγR1, FcγR2A, FcγR2B1, FcγR2B2, FcγR2C, FcγR3A, FcγR3B, FcεR2, FcεR1, FcRn, Fcα/μR and FcαR1.

Optionally, the intracellular spacer domain comprises at least one of an amino acid sequence comprising SEQ ID NO:054 and an amino acid sequence comprising SEQ ID NO:056.

Optionally, the chimeric antigen receptor further comprises an intracellular linker domain, and the intracellular linker domain connects the intracellular detection signaling domain with the intracellular activation signaling domain.

Optionally, the intracellular linker domain can provide the desired flexibility to allow for the desired expression, activity and/or conformational positioning of the chimeric antigen receptor. The intracellular linker domain can be of any suitable length to connect at least two domains of interest, and is preferably designed to be flexible enough to allow proper folding and/or function and/or activity of one or both domains to which it connects. The length of the intracellular linker domain is at least 3, 5, 10, 15, 20, 25, 30, 35, 40, 45, 50, 55, 60, 65, 70, 75, 80, 85, 90, 95, 90, 95 or 100 amino acids. In some embodiments, the length of peptide linker is about 0 to 200 amino acids, about 10 to 190 amino acids, about 20 to 180 amino acids, about 30 to 170 amino acids, about 40 to 160 amino acids, about 50 to 150 amino acids, about 60 to 140 amino acids, about 70 to 130 amino acids, about 80 to 120 amino acids, or about 90 to 110 amino acids. In some embodiments, the intracellular linker domain can comprise endogenous protein sequence. In some embodiments, the intracellular linker domain comprises glycine, alanine and/or serine residues. In some embodiments, the linker can comprise a motif, such as multiple or repeated motifs of GS, GGS, GGGGS, GGSG or SGGG. The intracellular linker domain can include any natural amino acid, non-natural amino acid, or a combination thereof.

Optionally, the intracellular linker domain comprises at least one of an amino acid sequence comprising SEQ ID NO:058, an amino acid sequence comprising SEQ ID NO:060, an amino acid sequence comprising SEQ ID NO:062, an amino acid sequence comprising SEQ ID NO:064, and an amino acid sequence comprising SEQ ID NO:066.

Optionally, the chimeric antigen receptor is an immune cell chimeric antigen receptor.

Optionally, the immune cell comprises T lymphocyte.

Optionally, the T lymphocyte comprises at least one of inflammatory T lymphocyte, cytotoxic T lymphocyte, regulatory T lymphocyte and helper T lymphocyte.

Optionally, the T lymphocyte comprises at least one of CD4⁺ T lymphocyte, CD8⁺ T lymphocyte, γδ T lymphocyte and NKT lymphocyte.

Optionally, the immune cell comprises phagocyte.

Optionally, the phagocyte comprises at least one of macrophage, monocyte, neutrophil, mast cell, dendritic cell and B cell.

Optionally, the chimeric antigen receptor comprising:
a) the extracellular target molecule binding domain comprising at least one of an amino acid sequence comprising SEQ ID NO:001, an amino acid sequence comprising SEQ ID NO:003, an amino acid sequence comprising SEQ ID NO:005, an amino acid sequence comprising SEQ ID NO:007, an amino acid sequence comprising SEQ ID NO:009, and an amino acid sequence comprising SEQ ID NO:011;
b) the transmembrane domain comprising at least one of an amino acid sequence comprising SEQ ID NO:012 and an amino acid sequence comprising SEQ ID NO:014;
c) an extracellular spacer domain through which the extracellular target molecule binding domain and the transmembrane domain are connected, wherein the extracellular spacer domain comprises at least one of an amino acid sequence comprising SEQ ID NO:016 and an amino acid sequence comprising SEQ ID NO:018; and
d) the intracellular signaling domain comprising at least one of an amino acid sequence comprising SEQ ID NO:020, an amino acid sequence comprising SEQ ID NO:022, an amino acid sequence comprising SEQ ID NO:024, an amino acid sequence comprising SEQ ID NO:026, an amino acid sequence comprising SEQ ID NO:028, an amino acid sequence comprising SEQ ID NO:030, an amino acid sequence comprising SEQ ID NO:032, an amino acid sequence comprising SEQ ID NO:034, an amino acid sequence comprising SEQ ID NO:036, an amino acid sequence comprising SEQ ID NO:038, an amino acid sequence comprising SEQ ID NO:040, an amino acid sequence comprising SEQ ID NO:042, an amino acid sequence comprising SEQ ID NO:044, an amino acid sequence comprising SEQ ID NO:046, an amino acid sequence comprising SEQ ID NO:048, an amino acid sequence comprising SEQ ID NO:050, and an amino acid sequence comprising SEQ ID NO:052.

Optionally, the chimeric antigen receptor comprising:
a) the extracellular target molecule binding domain comprising at least one of an amino acid sequence comprising SEQ ID NO:001, an amino acid sequence comprising SEQ ID NO:003, an amino acid sequence comprising SEQ ID NO:005, an amino acid sequence comprising SEQ ID NO:007, an amino acid sequence comprising SEQ ID NO:009, and an amino acid sequence comprising SEQ ID NO:011;
b) the transmembrane domain comprising at least one of an amino acid sequence comprising SEQ ID NO:012 and an amino acid sequence comprising SEQ ID NO:014;
c) an extracellular spacer domain through which the extracellular target molecule binding domain and the transmembrane domain are connected, wherein the extracellular spacer domain comprises at least one of an amino acid sequence comprising SEQ ID NO:016 and an amino acid sequence comprising SEQ ID NO:018; and
d) an intracellular activation signaling domain comprising at least one of an amino acid sequence comprising SEQ ID NO:042, an amino acid sequence comprising SEQ ID NO:044, an amino acid sequence comprising SEQ ID NO:046, an amino acid sequence comprising SEQ ID NO:048, an amino acid sequence comprising SEQ ID NO:050 and an amino acid sequence comprising SEQ ID NO:052.

Optionally, the chimeric antigen receptor comprising:
a) the extracellular target molecule binding domain comprising at least one of an amino acid sequence comprising SEQ ID NO:001, an amino acid sequence comprising SEQ ID NO:003, an amino acid sequence comprising SEQ ID NO:005, an amino acid sequence comprising SEQ ID NO:007, an amino acid sequence comprising SEQ ID NO:009, and an amino acid sequence comprising SEQ ID NO:011;
b) the transmembrane domain comprising at least one of an amino acid sequence comprising SEQ ID NO:012 and an amino acid sequence comprising SEQ ID NO:014;
c) an extracellular spacer domain through which the extracellular target molecule binding domain and the transmembrane domain are connected, wherein the extracellular spacer domain comprises at least one of an amino acid sequence comprising SEQ ID NO:016 and an amino acid sequence comprising SEQ ID NO:018;
d) an intracellular detection signaling domain comprising at least one of an amino acid sequence comprising SEQ ID NO:020, an amino acid sequence comprising SEQ ID NO:022, an amino acid sequence comprising SEQ ID NO:024, an amino acid sequence comprising SEQ ID NO:026, an amino acid sequence comprising SEQ ID NO:028, an amino acid sequence comprising SEQ ID NO:030, an amino acid sequence comprising SEQ ID NO:032, an amino acid sequence comprising SEQ ID NO:034, an amino acid sequence comprising SEQ ID NO:036, an amino acid sequence comprising SEQ ID NO:038, and an amino acid sequence comprising SEQ ID NO:040; and
e) an intracellular activation signaling domain comprising at least one of an amino acid sequence comprising SEQ ID NO:042, an amino acid sequence comprising SEQ ID NO:044, an amino acid sequence comprising SEQ ID NO:046, an amino acid sequence comprising SEQ ID NO:048, an amino acid sequence comprising SEQ ID NO:050 and an amino acid sequence comprising SEQ ID NO:052.

Optionally, the chimeric antigen receptor comprising:
a) the extracellular target molecule binding domain comprising at least one of an amino acid sequence comprising SEQ ID NO:001, an amino acid sequence comprising SEQ ID NO:003, an amino acid sequence comprising SEQ ID NO:005, an amino acid sequence comprising SEQ ID NO:007, an amino acid sequence comprising SEQ ID NO:009, and an amino acid sequence comprising SEQ ID NO:011;
b) the transmembrane domain comprising at least one of an amino acid sequence comprising SEQ ID NO:012 and an amino acid sequence comprising SEQ ID NO:014;
c) an extracellular spacer domain through which the extracellular target molecule binding domain and the transmembrane domain are connected, wherein the extracellular spacer domain comprises at least one of an amino acid sequence comprising SEQ ID NO:016 and an amino acid sequence comprising SEQ ID NO:018;
d) the intracellular detection signaling domain comprising at least one of an amino acid sequence comprising SEQ ID NO:020, an amino acid sequence comprising SEQ ID NO:022, an amino acid sequence comprising SEQ ID NO:024, an amino acid sequence comprising SEQ ID NO:026, an amino acid sequence comprising SEQ ID NO:028, an amino acid sequence comprising SEQ ID NO:030, an amino acid sequence comprising SEQ ID NO:032, an amino acid sequence comprising SEQ ID NO:034, an amino acid sequence comprising SEQ ID NO:036, an amino acid sequence comprising SEQ ID NO:038 and an amino acid sequence comprising SEQ ID NO:040;
e) an intracellular activation signaling domain comprising at least one of an amino acid sequence comprising SEQ ID NO:042, an amino acid sequence comprising SEQ ID NO:044, an amino acid sequence comprising SEQ ID NO:046, an amino acid sequence comprising SEQ ID NO:048, an amino acid sequence comprising SEQ ID NO:050 and an amino acid sequence comprising SEQ ID NO:052; and
f) an intracellular linker domain through which the intracellular detection signaling domain and the intracellular activation signaling domain are connected, wherein the intracellular linker domain comprises at least one of an amino acid sequence comprising SEQ ID NO: 058, an amino acid sequence comprising SEQ ID NO: 060, an amino acid sequence comprising SEQ ID NO: 062, an amino acid sequence comprising SEQ ID NO: 064 and an amino acid sequence comprising SEQ ID NO: 066.

Optionally, the chimeric antigen receptor comprising:
a) the extracellular target molecule binding domain comprising at least one of an amino acid sequence comprising SEQ ID NO:001, an amino acid sequence comprising SEQ ID NO:003, an amino acid sequence comprising SEQ ID NO:005, an amino acid sequence comprising SEQ ID NO:007, an amino acid sequence comprising SEQ ID NO:009, and an amino acid sequence comprising SEQ ID NO:011;
b) the transmembrane domain comprising at least one of an amino acid sequence comprising SEQ ID NO:012 and an amino acid sequence comprising SEQ ID NO:014;
c) an extracellular spacer domain through which the extracellular target molecule binding domain and the transmembrane domain are connected, wherein the extracellular spacer domain comprises at least one of an amino acid sequence comprising SEQ ID NO:016 and an amino acid sequence comprising SEQ ID NO:018;
d) an intracellular signaling domain comprising at least one of an amino acid sequence comprising SEQ ID NO:020, an amino acid sequence comprising SEQ ID NO:022, an amino acid sequence comprising SEQ ID NO:024, an amino acid sequence comprising SEQ ID NO:026, an amino acid sequence comprising SEQ ID NO:028, an amino acid sequence comprising SEQ ID NO:030, an amino acid sequence comprising SEQ ID NO:032, an amino acid sequence comprising SEQ ID NO:034, an amino acid sequence comprising SEQ ID NO:036, an amino acid sequence comprising SEQ ID NO:038, an amino acid sequence comprising SEQ ID NO:040, an amino acid sequence comprising SEQ ID NO:042, an amino acid sequence comprising SEQ ID NO:044, an amino acid sequence comprising SEQ ID NO:046, an amino acid sequence comprising SEQ ID NO:048, an amino acid sequence comprising SEQ ID NO:050 and an amino acid sequence comprising SEQ ID NO:052; and
e) an intracellular spacer domain through which the transmembrane domain and the intracellular signaling domain are connected, wherein the intracellular spacer domain comprises at least one of an amino acid sequence comprising SEQ ID NO:054 and an amino acid sequence comprising SEQ ID NO:056.

Optionally, the chimeric antigen receptor comprising:
a) an extracellular target molecule binding domain comprising at least one of an amino acid sequence comprising SEQ ID NO: 001, an amino acid sequence comprising SEQ ID NO: 003, an amino acid sequence comprising SEQ ID NO:005, an amino acid sequence comprising SEQ ID NO:007, at least one of an amino acid sequence comprising SEQ ID NO:009 and an amino acid sequence comprising SEQ ID NO:011;
b) the transmembrane domain comprising at least one of an amino acid sequence comprising SEQ ID NO:012 and an amino acid sequence comprising SEQ ID NO:014;
c) an extracellular spacer domain through which the extracellular target molecule binding domain and the transmembrane domain are connected, wherein the extracellular spacer domain comprises at least one of an amino acid sequence comprising SEQ ID NO:016 and an amino acid sequence comprising SEQ ID NO:018; and
d) an intracellular activation signaling domain comprising at least one of an amino acid sequence comprising SEQ ID NO:042, an amino acid sequence comprising SEQ ID NO:044, an amino acid sequence comprising SEQ ID NO:046, an amino acid sequence comprising SEQ ID NO:048, an amino acid sequence comprising SEQ ID NO:050 and an amino acid sequence comprising SEQ ID NO:052; and
e) an intracellular spacer domain through which the transmembrane domain and the intracellular activation signaling domain are connected, wherein the intracellular spacer domain comprises at least one of an amino acid sequence comprising SEQ ID NO:054 and an amino acid sequence comprising SEQ ID NO:056.

Optionally, the chimeric antigen receptor comprising:
a) the extracellular target molecule binding domain comprising at least one of an amino acid sequence comprising SEQ ID NO:001, an amino acid sequence comprising SEQ ID NO:003, an amino acid sequence comprising SEQ ID NO:005, an amino acid sequence comprising SEQ ID NO:007, an amino acid sequence comprising SEQ ID NO:009, and an amino acid sequence comprising SEQ ID NO:011;
b) the transmembrane domain comprising at least one of an amino acid sequence comprising SEQ ID NO:012 and an amino acid sequence comprising SEQ ID NO:014;
c) an extracellular spacer domain through which the extracellular target molecule binding domain and the transmembrane domain are connected, wherein the extracellular spacer domain comprises at least one of an amino acid sequence comprising SEQ ID NO:016 and an amino acid sequence comprising SEQ ID NO:018;
d) the intracellular detection signaling domain comprising at least one of an amino acid sequence comprising SEQ ID NO:020, an amino acid sequence comprising SEQ ID NO:022, an amino acid sequence comprising SEQ ID NO:024, an amino acid sequence comprising SEQ ID NO:026, an amino acid sequence comprising SEQ ID NO:028, an amino acid sequence comprising SEQ ID NO:030, an amino acid sequence comprising SEQ ID NO:032, an amino acid sequence comprising SEQ ID NO:034, an amino acid sequence comprising SEQ ID NO:036, an amino acid sequence comprising SEQ ID NO:038 and an amino acid sequence comprising SEQ ID NO: 040;
e) an intracellular activation signaling domain comprising at least one of an amino acid sequence comprising SEQ ID NO:042, an amino acid sequence comprising SEQ ID NO:044, an amino acid sequence comprising SEQ ID NO:046, an amino acid sequence comprising SEQ ID NO:048, an amino acid sequence comprising SEQ ID NO:050 and an amino acid sequence comprising SEQ ID NO:052; and
f) an intracellular spacer domain through which the transmembrane domain and the intracellular detection signaling domain are connected, wherein the intracellular spacer domain comprises at least one of an amino acid sequence comprising SEQ ID NO:054 and an amino acid sequence comprising SEQ ID NO:056.

Optionally, the chimeric antigen receptor comprising:
a) the extracellular target molecule binding domain comprising at least one of an amino acid sequence comprising SEQ ID NO:001, an amino acid sequence comprising SEQ ID NO:003, an amino acid sequence comprising SEQ ID NO:005, an amino acid sequence comprising SEQ ID NO:007, an amino acid sequence comprising SEQ ID NO:009, and an amino acid sequence comprising SEQ ID NO:011;
b) the transmembrane domain comprising at least one of an amino acid sequence comprising SEQ ID NO:012 and an amino acid sequence comprising SEQ ID NO:014;
c) an extracellular spacer domain through which the extracellular target molecule binding domain and the transmembrane domain are connected, wherein the extracellular spacer domain comprises at least one of an amino acid sequence comprising SEQ ID NO:016 and an amino acid sequence comprising SEQ ID NO:018;
d) the intracellular detection signaling domain comprising at least one of an amino acid sequence comprising SEQ ID NO:020, an amino acid sequence comprising SEQ ID NO:022, an amino acid sequence comprising SEQ ID NO:024, an amino acid sequence comprising SEQ ID NO:026, an amino acid sequence comprising SEQ ID NO:028, an amino acid sequence comprising SEQ ID NO:030, an amino acid sequence comprising SEQ ID NO:032, an amino acid sequence comprising SEQ ID NO:034, an amino acid sequence comprising SEQ ID NO:036, an amino acid sequence comprising SEQ ID NO:038 and an amino acid sequence comprising SEQ ID NO:040;
e) an intracellular activation signaling domain comprising at least one of an amino acid sequence comprising SEQ ID NO:042, an amino acid sequence comprising SEQ ID NO:044, an amino acid sequence comprising SEQ ID NO:046, an amino acid sequence comprising SEQ ID NO:048, an amino acid sequence comprising SEQ ID NO:050 and an amino acid sequence comprising SEQ ID NO:052;
f) an intracellular spacer domain through which the transmembrane domain and the intracellular detection signaling domain are connected, wherein the intracellular spacer domain comprises at least one of an amino acid sequence comprising SEQ ID NO:054 and an amino acid sequence comprising SEQ ID NO:056; and
g) an intracellular linker domain through which the intracellular detection signaling domain and the intracellular activation signaling domain are connected, wherein the intracellular linker domain comprises at least one of an amino acid sequence comprising SEQ ID NO:058, an amino acid sequence comprising SEQ ID NO:060, an amino acid sequence comprising SEQ ID NO:062, an amino acid sequence comprising SEQ ID NO:064 and an amino acid sequence comprising SEQ ID NO:066.

As an embodiment, the chimeric antigen receptor comprising:
a) an extracellular target molecule binding domain for specifically binding to a target molecule;
b) an intracellular detection signaling domain which is at least one of CD3ζ ITAM1 fragment, CD3ζ ITAM2 fragment, CD3ζ ITAM3 fragment, FcRIIA ITAM fragment, FcRγ ITAM fragment, DAP12 ITAM fragment and CD3ε ITAM fragment;
c) an intracellular signaling domain connected to the intracellular detection signaling domain; and
d) a transmembrane domain, used to connect the extracellular target molecule binding domain and the intracellular signaling domain, and anchor these two domains on a cell membrane.

Optionally, the intracellular signaling domain comprises at least one intracellular activation signaling domain, and an activation of the intracellular activation signaling domain at least relies on the binding of the extracellular target molecule binding domain to the target molecule, and the intracellular activation signaling domain contains a molecule having a catalytic domain.

Optionally, the chimeric antigen receptor is an immune cell chimeric antigen receptor.

Optionally, the immune cell comprises T lymphocyte.

Optionally, the T lymphocyte comprises at least one of inflammatory T lymphocyte, cytotoxic T lymphocyte, regulatory T lymphocyte and helper T lymphocyte.

Optionally, the T lymphocyte comprises at least one of CD4⁺ T lymphocyte, CD8⁺ T lymphocyte, γδ T lymphocyte and NKT lymphocyte.

Optionally, the immune cell comprises phagocyte.

Optionally, the phagocyte comprises at least one of macrophage, monocyte, neutrophil, mast cell, dendritic cell and B cell.

Table 1 refers to the amino acid sequence and nucleic acid sequence

TABLE 1

| Numbering | Nucleic acid sequence | Name | Amino acid sequence |
|---|---|---|---|
| SEQ ID | | Extracellular target | PPTFSPALLVVTEGDNATFTCSFSNTSES |
| NO: 001 | | molecule binding | FVLNWYRMSPSNQTDKLAAFPEDRSQ |
| | | domain (“Extracellular | PGQDCRFRVTQLPNGRDFHMSVVRAR |
| | | target molecule | RNDSGTYLCGAISLAPKAQIKESLRAEL |
| | | binding domain in | RVTERRAE |
| | | Truncated PD-1” in | |
| | | the Examples and | |
| | | “Extracellular target | |
| | | molecule binding | |
| | | domain in Full-length | |
| | | PD-1” in the | |
| | | Examples) | |
| SEQ ID | CCCCCCACCTTCTCCCCAGCCCTGCTCGTGGTGACCGAAGGGGACAACGCCACCTTCACC | Extracellular target | / |
| NO: 002 | TGCAGCTTCTCCAACACATCGGAGAGCTTCGTGCTAAACTGGTACCGCATGAGCCCCAGC | molecule binding | |
| | AACCAGACGGACAAGCTGGCCGCCTTCCCCGAGGACCGCAGCCAGCCCGGCCAGGACTG | domain (“Extracellular | |
| | CCGCTTCCGTGTCACACAACTGCCCAACGGGCGTGACTTCCACATGAGCGTGGTCAGGGC | target molecule | |
| | CCGGCGCAATGACAGCGGCACCTACCTCTGTGGGGCCATCTCCCTGGCCCCCAAGGCGCA | binding domain in | |
| | GATCAAAGAGAGCCTGCGGGCAGAGCTCAGGGTGACAGAGAGAAGGGCAGAA | Truncated PD-1” in | |
| | | the Examples and | |
| | | “Extracellular target | |
| | | molecule binding | |
| | | domain in Full-length | |
| | | PD-1” in the | |
| | | Examples) | |
| SEQ ID | | Extracellular target | PTFSPALLVVTEGDNATFTCSFSNTSESF |
| NO: 003 | | molecule binding | VLNWYRMSPSNQTDKLAAFPEDRSQP |
| | | domain | GQDCRFRVTQLPNGRDFHMSVVRARR |
| | | | NDSGTYLCGAISLAPKAQIKESLRAELR |
| | | | V |
| SEQ ID | CCCACCTTCTCCCCAGCCCTGCTCGTGGTGACCGAAGGGGACAACGCCACCTTCACCTGC | Extracellular target | / |
| NO: 004 | AGCTTCTCCAACACATCGGAGAGCTTCGTGCTAAACTGGTACCGCATGAGCCCCAGCAAC | molecule binding | |
| | CAGACGGACAAGCTGGCCGCCTTCCCCGAGGACCGCAGCCAGCCCGGCCAGGACTGCCG | domain | |
| | CTTCCGTGTCACACAACTGCCCAACGGGCGTGACTTCCACATGAGCGTGGTCAGGGCCCG | | |
| | GCGCAATGACAGCGGCACCTACCTCTGTGGGGCCATCTCCCTGGCCCCCAAGGCGCAGAT | | |
| | CAAAGAGAGCCTGCGGGCAGAGCTCAGGGTG | | |
| SEQ ID | | Extracellular target | FLDSPDRPWNPPTFSPALLVVTEGDNAT |
| NO: 005 | | molecule binding | FTCSFSNTSESFVLNWYRMSPSNQTDK |
| | | domain | LAAFPEDRSQPGQDCRFRVTQLPNGRD |
| | | | FHMSVVRARRNDSGTYLCGAISLAPKA |
| | | | QIKESLRAELRV |
| SEQ ID | TTCTTAGACTCCCCAGACAGGCCCTGGAACCCCCCCACCTTCTCCCCAGCCCTGCTCGTG | Extracellular target | / |
| NO: 006 | GTGACCGAAGGGGACAACGCCACCTTCACCTGCAGCTTCTCCAACACATCGGAGAGCTT | molecule binding | |
| | CGTGCTAAACTGGTACCGCATGAGCCCCAGCAACCAGACGGACAAGCTGGCCGCCTTCC | domain | |
| | CCGAGGACCGCAGCCAGCCCGGCCAGGACTGCCGCTTCCGTGTCACACAACTGCCCAAC | | |
| | GGGCGTGACTTCCACATGAGCGTGGTCAGGGCCCGGCGCAATGACAGCGGCACCTACCT | | |
| | CTGTGGGGCCATCTCCCTGGCCCCCAAGGCGCAGATCAAAGAGAGCCTGCGGGCAGAGC | | |
| | TCAGGGTG | | |
| SEQ ID | | Extracellular target | QVQLQQSGPGLVKPSQTLSLTCAISGDS |
| NO: 007 | | molecule binding | VSSTKAAWYWIRQSPSRGLEWLGRTYF |
| | | domain | RSKWYNDYADSVKSRLTINPDTSKNQF |
| | | | SLQLKSVSPEDTAVYYCARGQYTAFDI |
| | | | WGQGTMVTVSSGILGSGGGGSGGGGS |
| | | | GGGGSQSALIQPASVSGSPGQSITISCTG |
| | | | TSSDVGGYDLVSWYQQYPGQAPRLIIY |
| | | | EVIKRPSGISDRFSGSKSGNTASLTISGL |
| | | | QAEDEADYYCCSYAGRRLHGVFGGGT |
| | | | QLTVL |
| SEQ ID | CAGGTACAGCTGCAGCAGTCAGGTCCAGGACTGGTGAAGCCCTCGCAGACCCTCTCACT | Extracellular target | / |
| NO: 008 | CACCTGTGCCATCTCCGGGGACAGTGTCTCTAGCACCAAGGCTGCTTGGTACTGGATCAG | molecule binding | |
| | GCAGTCCCCTTCGAGAGGCCTTGAGTGGCTGGGAAGGACATACTTCCGGTCCAAGTGGTA | domain | |
| | TAATGACTATGCCGACTCTGTGAAAAGTCGATTAACCATCAACCCAGACACATCCAAGAA | | |
| | CCAGTTCTCCCTGCAACTTAAGTCTGTGAGTCCCGAGGACACGGCTGTGTATTACTGTGC | | |
| | AAGAGGGCAATACACTGCTTTTGATATCTGGGGCCAAGGGACAATGGTCACCGTCTCTTC | | |
| | AGGAATTCTAGGATCCGGTGGCGGTGGCAGCGGCGGTGGTGGTTCCGGAGGCGGCGGTT | | |
| | CTCAGTCTGCTCTGATTCAGCCTGCCTCCGTGTCTGGGTCCCCTGGACAGTCGATCACTAT | | |
| | CTCCTGTACTGGCACCAGTAGTGATGTTGGAGGTTATGACCTTGTCTCCTGGTACCAACAG | | |
| | TACCCGGGCCAAGCCCCCAGACTCATCATTTATGAGGTCATTAAGCGGCCCTCAGGGATTT | | |
| | CTGATCGCTTCTCTGGTTCCAAGTCTGGCAACACGGCCTCCCTGACAATCTCTGGGCTCCA | | |
| | GGCTGAGGACGAGGCTGATTATTATTGCTGCTCATATGCAGGTAGACGTCTTCATGGTGTG | | |
| | TTCGGAGGAGGCACCCAGCTGACCGTCCTC | | |
| SEQ ID | | Extracellular target | QSALTQPASVSGSPGQSITISCTGTSSDV |
| NO: 009 | | molecule binding | GGYNYVSWYQQHPGKAPKLMIYDVS |
| | | domain | NRPSGVSNRFSGSKSGNTASLTISGLQA |
| | | | EDEADYYCSSYTSSSTRVFGTGTKVTV |
| | | | LGGGGSGGGGSGGGGSEVQLLESGGG |
| | | | LVQPGGSLRLSCAASGFTFSSYIMMWV |
| | | | RQAPGKGLEWVSSIYPSGGITFYADTV |
| | | | KGRFTISRDNSKNTLYLQMNSLRAEDT |
| | | | AVYYCARIKLGTVTTVDYWGQGTLVT |
| | | | VSS |
| SEQ ID | CAGTCCGCCCTGACCCAGCCTGCCTCCGTGTCTGGCTCCCCTGGCCAGTCCATCACCATC | Extracellular target | / |
| NO: 010 | AGCTGCACCGGCACCTCCAGCGACGTGGGCGGCTACAACTACGTGTCCTGGTATCAGCAG | molecule binding | |
| | CACCCCGGCAAGGCCCCCAAGCTGATGATCTACGACGTGTCCAACCGGCCCTCCGGCGTG | domain | |
| | TCCAACAGATTCTCCGGCTCCAAGTCCGGCAACACCGCCTCCCTGACCATCAGCGGACTG | | |
| | CAGGCAGAGGACGAGGCCGACTACTACTGCTCCTCCTACACCTCCTCCAGCACCAGAGTG | | |
| | TTCGGCACCGGCACAAAAGTGACCGTGCTGGGAGGGGGCGGTTCCGGAGGAGGCGGCA | | |
| | GCGGGGGAGGAGGTAGCGAGGTGCAGCTGCTGGAATCCGGCGGAGGACTGGTGCAGCC | | |
| | TGGCGGCTCCCTGAGACTGTCTTGCGCCGCCTCCGGCTTCACCTTCTCCAGCTACATCATG | | |
| | ATGTGGGTGCGACAGGCCCCTGGCAAGGGCCTGGAATGGGTGTCCTCCATCTACCCCTCC | | |
| | GGCGGCATCACCTTCTACGCCGACACCGTGAAGGGCCGGTTCACCATCTCCCGGGACAAC | | |
| | TCCAAGAACACCCTGTACCTGCAGATGAACTCCCTGCGGGCCGAGGACACCGCCGTGTA | | |
| | CTACTGCGCCCGGATCAAGCTGGGCACCGTGACCACCGTGGACTACTGGGGCCAGGGCA | | |
| | CCCTGGTGACAGTGTCCTCC | | |
| SEQ ID | | Extracellular target | EIVMTQSPSTLSASVGDRVIITCQASEDI |
| NO: 011 | | molecule binding | YSLLAWYQQKPGKAPKLLIYDASDLAS |
| | | domain | GVPSRFSGSGSGAEFTLTISSLQPDDFAT |
| | | | YYCQGNYGSSSSSSYGAVFGQGTKLTV |
| | | | LGGGGGSGGGGSGGGGSGGGGSEVQL |
| | | | VESGGGLVQPGGSLRLSCTVSGIDLSSY |
| | | | TMGWVRQAPGKGLEWVGIISSGGRTY |
| | | | YASWAKGRFTISRDTSKNTVYLQMNSL |
| | | | RAEDTAVYYCARGRYTGYPYYFALWG |
| | | | QGTLVTVSS |
| SEQ ID | | Transmembrane | VGVVGGLLGSLVLLVWVLAVI |
| NO: 012 | | domain | |
| | | (“Transmembrane | |
| | | domain in Truncated | |
| | | PD-1” in the Examples | |
| | | and “Transmembrane | |
| | | domain in Full-length | |
| | | PD-1” in the | |
| | | Examples) | |
| SEQ ID | GTTGGTGTCGTGGGCGGCCTGCTGGGCAGCCTGGTGCTGCTAGTCTGGGTCCTGGCCGTC | Transmembrane | / |
| NO: 013 | ATC | domain | |
| | | (“Transmembrane | |
| | | domain in Truncated | |
| | | PD-1” in the Examples | |
| | | and “Transmembrane | |
| | | domain in Full-length | |
| | | PD-1” in the | |
| | | Examples) | |
| SEQ ID | | Transmembrane | TLVVGVVGGLLGSLVLLVWVLAVI |
| NO: 014 | | domain | |
| SEQ ID | ACCCTGGTGGTTGGTGTCGTGGGCGGCCTGCTGGGCAGCCTGGTGCTGCTAGTCTGGGTC | Transmembrane | / |
| NO: 015 | CTGGCCGTCATC | domain | |
| SEQ ID | | Extracellular spacer | VPTAHPSPSPRPAGQFQTLV |
| NO: 016 | | domain (“Extracellular | |
| | | spacer domain in | |
| | | Truncated PD-1” in | |
| | | the Examples and | |
| | | “Extracellular spacer | |
| | | domain in Full-length | |
| | | PD-1” in the | |
| | | Examples) | |
| SEQ ID | GTGCCCACAGCCCACCCCAGCCCCTCACCCAGGCCAGCCGGCCAGTTCCAAACCCTGGT | Extracellular spacer | / |
| NO: 017 | G | domain (“Extracellular | |
| | | spacer domain in | |
| | | Truncated PD-1” in | |
| | | the Examples and | |
| | | “Extracellular spacer | |
| | | domain in Full-length | |
| | | PD-1” in the | |
| | | Examples) | |
| SEQ ID | | Extracellular spacer | TERRAEVPTAHPSPSPRPAGQFQ |
| NO: 018 | | domain | |
| SEQ ID | ACAGAGAGAAGGGCAGAAGTGCCCACAGCCCACCCCAGCCCCTCACCCAGGCCAGCCG | Extracellular spacer | / |
| NO: 019 | GCCAGTTCCAA | domain | |
| SEQ ID | | Intracellular detection | NQLYNELNLGRREEYDVLDK |
| NO: 020 | | signaling domain | |
| | | (“Sub1” in the | |
| | | examples) | |
| SEQ ID | AACCAGCTCTATAACGAGCTCAATCTAGGACGAAGAGAGGAGTACGATGTTTTGGACAAG | Intracellular detection | / |
| NO: 021 | | signaling domain | |
| | | (“Sub1” in the | |
| | | examples) | |
| SEQ ID | | Intracellular detection | NQLFNELNLGRREEFDVLDK |
| NO: 022 | | signaling domain | |
| | | (“Sub1FF” in the | |
| | | examples) | |
| SEQ ID | AACCAGCTCTTTAACGAGCTCAATCTAGGACGAAGAGAGGAGTTCGATGTTTTGGACAAG | Intracellular detection | / |
| NO: 023 | | signaling domain | |
| | | (“Sub1FF” in the | |
| | | examples) | |
| SEQ ID | | Intracellular detection | EGLYNELQKDKMAEAYSEIGM |
| NO: 024 | | signaling domain | |
| | | (“Sub2” in the | |
| | | examples) | |
| SEQ ID | GAAGGCCTGTACAATGAACTGCAGAAAGATAAGATGGCGGAGGCCTACAGTGAGATTGG | Intracellular detection | / |
| NO: 025 | GATG | signaling domain | |
| | | (“Sub2” in the | |
| | | examples) | |
| SEQ ID | | Intracellular detection | EGLFNELQKDKMAEAFSEIGM |
| NO: 026 | | signaling domain | |
| | | (“Sub2FF” in the | |
| | | examples) | |
| SEQ ID | GAAGGCCTGTTCAATGAACTGCAGAAAGATAAGATGGCGGAGGCCTTCAGTGAGATTGG | Intracellular detection | / |
| NO: 027 | GATG | signaling domain | |
| | | (“Sub2FF” in the | |
| | | examples) | |
| SEQ ID | | Intracellular detection | DGLYQGLSTATKDTYDALHM |
| NO: 028 | | signaling domain | |
| | | (“Sub3” in the | |
| | | examples) | |
| SEQ ID | GATGGCCTTTACCAGGGACTCAGTACAGCCACCAAGGACACCTACGACGCCCTTCACATG | Intracellular detection | / |
| NO: 029 | | signaling domain | |
| | | (“Sub3” in the | |
| | | examples) | |
| SEQ ID | | Intracellular detection | DGLFQGLSTATKDTFDALHM |
| NO: 030 | | signaling domain | |
| | | (“Sub3FF” in the | |
| | | examples) | |
| SEQ ID | GATGGCCTTTTCCAGGGACTCAGTACAGCCACCAAGGACACCTTCGACGCCCTTCACATG | Intracellular detection | / |
| NO: 031 | | signaling domain | |
| | | (“Sub3FF” in the | |
| | | examples) | |
| SEQ ID | | Intracellular detection | PDYEPIRKGQRDLYSGLNQR |
| NO: 032 | | signaling domain | |
| | | (“Sub4” in the | |
| | | examples) | |
| SEQ ID | CCAGACTATGAGCCCATCCGGAAAGGCCAGCGGGACCTGTATTCTGGCCTGAATCAGAGA | Intracellular detection | / |
| NO: 033 | | signaling domain | |
| | | (“Sub4” in the | |
| | | examples) | |
| SEQ ID | | Intracellular detection | PDFEPIRKGQRDLFSGLNQR |
| NO: 034 | | signaling domain | |
| | | (“Sub4FF” in the | |
| | | examples) | |
| SEQ ID | CCAGACTTTGAGCCCATCCGGAAAGGCCAGCGGGACCTGTTTTCTGGCCTGAATCAGAGA | Intracellular detection | / |
| NO: 035 | | signaling domain | |
| | | (“Sub4FF” in the | |
| | | examples) | |
| SEQ ID | | Intracellular detection | GGYMTLNPRAPTDDDKNIYLTLPPNGT |
| NO: 036 | | signaling domain | |
| | | (“Sub5” in the | |
| | | examples) | |
| SEQ ID | GGCGGCTACATGACTCTGAACCCCAGGGCACCTACTGACGATGATAAAAACATCTACCTG | Intracellular detection | / |
| NO: 037 | ACTCTTCCTCCCAACGGTACC | signaling domain | |
| | | (“Sub5” in the | |
| | | examples) | |
| SEQ ID | | Intracellular detection | GVYTGLSTRNQETYETLKHE |
| NO: 038 | | signaling domain | |
| | | (“Sub6” in the | |
| | | examples) | |
| SEQ ID | GGTGTTTACACGGGCCTGAGCACCAGGAACCAGGAGACTTACGAGACTCTGAAGCATGA | Intracellular detection | / |
| NO: 039 | G | signaling domain | |
| | | (“Sub6” in the | |
| | | examples) | |
| SEQ ID | | Intracellular detection | SPYQELQGQRSDVYSDLNTQGT |
| NO: 040 | | signaling domain | |
| | | (“Sub7” in the | |
| | | examples) | |
| SEQ ID | TCGCCTTATCAGGAACTCCAGGGTCAGAGGTCGGATGTCTACAGCGACCTCAACACACAG | Intracellular detection | / |
| NO: 041 | GGTACC | signaling domain | |
| | | (“Sub7” in the | |
| | | examples) | |
| SEQ ID | | Intracellular activation | PDPAAHLPFFYGSISRAEAEEHLKLAG |
| NO: 042 | | signaling domain | MADGLFLLRQCLRSLGGYVLSLVHDV |
| | | (“ZAP70” in the | RFHHFPIERQLNGTYAIAGGKAHCGPA |
| | | examples) | ELCEFYSRDPDGLPCNLRKPCNRPSGLE |
| | | | PQPGVFDCLRDAMVRDYVRQTWKLE |
| | | | GEALEQAIISQAPQVEKLIATTAHERMP |
| | | | WYHSSLTREEAERKLYSGAQTDGKFLL |
| | | | RPRKEQGTYALSLIYGKTVYHYLISQD |
| | | | KAGKYCIPEGTKFDTLWQLVEYLKLKA |
| | | | DGLIYCLKEACPNSSASNASGAAAPTLP |
| | | | AHPSTLTHPQRRIDTLNSDGYTPEPARIT |
| | | | SPDKPRPMPMDTSVYESPYSDPEELKD |
| | | | KKLFLKRDNLLIADIELGCGNFGSVRQ |
| | | | GVYRMRKKQIDVAIKVLKQGTEKADT |
| | | | EEMMREAQIMHQLDNPYIVRLIGVCQA |
| | | | EALMLVMEMAGGGPLHKFLVGKREEI |
| | | | PVSNVAELLHQVSMGMKYLEEKNFVH |
| | | | RDLAARNVLLVNRHYAKISDFGLSKAL |
| | | | GADDSYYTARSAGKWPLKWYAPECIN |
| | | | FRKFSSRSDVWSYGVTMWEALSYGQK |
| | | | PYKKMKGPEVMAFIEQGKRMECPPEC |
| | | | PPELYALMSDCWIYKWEDRPDFLTVEQ |
| | | | RMRACYYSLASKVEGPPGSTQKAEAA |
| | | | CA |
| SEQ ID | CCAGACCCCGCGGCGCACCTGCCCTTCTTCTACGGCAGCATCTCGCGTGCCGAGGCCGAG | Intracellular activation | / |
| NO: 043 | GAGCACCTGAAGCTGGCGGGCATGGCGGACGGGCTCTTCCTGCTGCGCCAGTGCCTGCG | signaling domain | |
| | CTCGCTGGGCGGCTATGTGCTGTCGCTCGTGCACGATGTGCGCTTCCACCACTTTCCCATC | (“ZAP70” in the | |
| | GAGCGCCAGCTCAACGGCACCTACGCCATTGCCGGCGGCAAAGCGCACTGTGGACCGGC | examples) | |
| | AGAGCTCTGCGAGTTCTACTCGCGCGACCCCGACGGGCTGCCCTGCAACCTGCGCAAGC | | |
| | CGTGCAACCGGCCGTCGGGCCTCGAGCCGCAGCCGGGGGTCTTCGACTGCCTGCGAGAC | | |
| | GCCATGGTGCGTGACTACGTGCGCCAGACGTGGAAGCTGGAGGGCGAGGCCCTGGAGCA | | |
| | GGCCATCATCAGCCAGGCCCCGCAGGTGGAGAAGCTCATTGCTACGACGGCCCACGAGC | | |
| | GGATGCCCTGGTACCACAGCAGCCTGACGCGTGAGGAGGCCGAGCGCAAACTTTACTCT | | |
| | GGGGCGCAGACCGACGGCAAGTTCCTGCTGAGGCCGCGGAAGGAGCAGGGCACATACG | | |
| | CCCTGTCCCTCATCTATGGGAAGACGGTGTACCACTACCTCATCAGCCAAGACAAGGCGG | | |
| | GCAAGTACTGCATTCCCGAGGGCACCAAGTTTGACACGCTCTGGCAGCTGGTGGAGTATC | | |
| | TGAAGCTGAAGGCGGACGGGCTCATCTACTGCCTGAAGGAGGCCTGCCCCAACAGCAGT | | |
| | GCCAGCAACGCCTCAGGGGCTGCTGCTCCCACACTCCCAGCCCACCCATCCACGTTGACT | | |
| | CATCCTCAGAGACGAATCGACACCCTCAACTCAGATGGATACACCCCTGAGCCAGCACGC | | |
| | ATAACGTCCCCAGACAAACCGCGGCCGATGCCCATGGACACGAGCGTGTATGAGAGCCCC | | |
| | TACAGCGACCCAGAGGAGCTCAAGGACAAGAAGCTCTTCCTGAAGCGCGATAACCTCCT | | |
| | CATAGCTGACATTGAACTTGGCTGCGGCAACTTTGGCTCAGTGCGCCAGGGCGTGTACCG | | |
| | CATGCGCAAGAAGCAGATCGACGTGGCCATCAAGGTGCTGAAGCAGGGCACGGAGAAG | | |
| | GCAGACACGGAAGAGATGATGCGCGAGGCGCAGATCATGCACCAGCTGGACAACCCCTA | | |
| | CATCGTGCGGCTCATTGGCGTCTGCCAGGCCGAGGCCCTCATGCTGGTCATGGAGATGGC | | |
| | TGGGGGCGGGCCGCTGCACAAGTTCCTGGTCGGCAAGAGGGAGGAGATCCCTGTGAGCA | | |
| | ATGTGGCCGAGCTGCTGCACCAGGTGTCCATGGGGATGAAGTACCTGGAGGAGAAGAAC | | |
| | TTTGTGCACCGTGACCTGGCGGCCCGCAACGTCCTGCTGGTTAACCGGCACTACGCCAAG | | |
| | ATCAGCGACTTTGGCCTCTCCAAAGCACTGGGTGCCGACGACAGCTACTACACTGCCCGC | | |
| | TCAGCAGGGAAGTGGCCGCTCAAGTGGTACGCACCCGAATGCATCAACTTCCGCAAGTTC | | |
| | TCCAGCCGCAGCGATGTCTGGAGCTATGGGGTCACCATGTGGGAGGCCTTGTCCTACGGC | | |
| | CAGAAGCCCTACAAGAAGATGAAAGGGCCGGAGGTCATGGCCTTCATCGAGCAGGGCAA | | |
| | GCGGATGGAGTGCCCACCAGAGTGTCCACCCGAACTGTACGCACTCATGAGTGACTGCTG | | |
| | GATCTACAAGTGGGAGGATCGCCCCGACTTCCTGACCGTGGAGCAGCGCATGCGAGCCTG | | |
| | TTACTACAGCCTGGCCAGCAAGGTGGAAGGGCCCCCAGGCAGCACACAGAAGGCTGAG | | |
| | GCTGCCTGTGCC | | |
| | | Intracellular activation | ASSGMADSANHLPFFFGNITREEAEDY |
| SEQ ID | | signaling domain | LVQGGMSDGLYLLRQSRNYLGGFALSV |
| NO: 044 | | (“SYK” in the | AHGRKAHHYTIERELNGTYAIAGGRTH |
| | | examples) | ASPADLCHYHSQESDGLVCLLKKPFNR |
| | | | PQGVQPKTGPFEDLKENLIREYVKQTW |
| | | | NLQGQALEQAIISQKPQLEKLIATTAHE |
| | | | KMPWFHGKISREESEQIVLIGSKTNGKF |
| | | | LIRARDNNGSYALCLLHEGKVLHYRID |
| | | | KDKTGKLSIPEGKKFDTLWQLVEHYSY |
| | | | KADGLLRVLTVPCQKIGTQGNVNFGGR |
| | | | PQLPGSHPATWSAGGIISRIKSYSFPKPG |
| | | | HRKSSPAQGNRQESTVSFNPYEPELAP |
| | | | WAADKGPQREALPMDTEVYESPYADP |
| | | | EEIRPKEVYLDRKLLTLEDKELGSGNFG |
| | | | TVKKGYYQMKKVVKTVAVKILKNEAN |
| | | | DPALKDELLAEANVMQQLDNPYIVRMI |
| | | | GICEAESWMLVMEMAELGPLNKYLQQ |
| | | | NRHVKDKNIIELVHQVSMGMKYLEES |
| | | | NFVHRDLAARNVLLVTQHYAKISDFGL |
| | | | SKALRADENYYKAQTHGKWPVKWYA |
| | | | PECINYYKFSSKSDVWSFGVLMWEAFS |
| | | | YGQKPYRGMKGSEVTAMLEKGERMG |
| | | | CPAGCPREMYDLMNLCWTYDVENRPG |
| | | | FAAVELRLRNYYYDVVN |
| SEQ ID | GCCAGCAGCGGCATGGCTGACAGCGCCAACCACCTGCCCTTCTTTTTCGGCAACATCACC | Intracellular activation | / |
| NO: 045 | CGGGAGGAGGCAGAAGATTACCTGGTCCAGGGGGGCATGAGTGATGGGCTTTATTTGCTG | signaling domain | |
| | CGCCAGAGCCGCAACTACCTGGGTGGCTTCGCCCTGTCCGTGGCCCACGGGAGGAAGGC | (“SYK” in the | |
| | ACACCACTACACCATCGAGCGGGAGCTGAATGGCACCTACGCCATCGCCGGTGGCAGGA | examples) | |
| | CCCATGCCAGCCCCGCCGACCTCTGCCACTACCACTCCCAGGAGTCTGATGGCCTGGTCT | | |
| | GCCTCCTCAAGAAGCCCTTCAACCGGCCCCAAGGGGTGCAGCCCAAGACTGGGCCCTTT | | |
| | GAGGATTTGAAGGAAAACCTCATCAGGGAATATGTGAAGCAGACATGGAACCTGCAGGG | | |
| | TCAGGCTCTGGAGCAGGCCATCATCAGTCAGAAGCCTCAGCTGGAGAAGCTGATCGCTAC | | |
| | CACAGCCCATGAAAAAATGCCTTGGTTCCATGGAAAAATCTCTCGGGAAGAATCTGAGCA | | |
| | AATTGTCCTGATAGGATCAAAGACAAATGGAAAGTTCCTGATCCGAGCCAGAGACAACAA | | |
| | CGGCTCCTACGCCCTGTGCCTGCTGCACGAAGGGAAGGTGCTGCACTATCGCATCGACAA | | |
| | AGACAAGACAGGGAAGCTCTCCATCCCCGAGGGAAAGAAGTTCGACACGCTCTGGCAGC | | |
| | TAGTCGAGCATTATTCTTATAAAGCAGATGGTTTGTTAAGAGTTCTTACTGTCCCATGTCAA | | |
| | AAAATCGGCACACAGGGAAATGTTAATTTTGGAGGCCGTCCACAACTTCCAGGTTCCCAT | | |
| | CCTGCGACTTGGTCAGCGGGTGGAATAATCTCAAGAATCAAATCATACTCCTTCCCAAAG | | |
| | CCTGGCCACAGAAAGTCCTCCCCTGCCCAAGGGAACCGGCAAGAGAGTACTGTGTCATT | | |
| | CAATCCGTATGAGCCAGAACTTGCACCCTGGGCTGCAGACAAAGGCCCCCAGAGAGAAG | | |
| | CCCTACCCATGGACACAGAGGTGTACGAGAGCCCCTACGCGGACCCCGAGGAGATCAGG | | |
| | CCCAAGGAGGTTTACCTGGACCGAAAGCTGCTGACGCTGGAAGACAAAGAACTGGGCTC | | |
| | TGGTAATTTTGGAACTGTGAAAAAGGGCTACTACCAAATGAAAAAAGTTGTGAAAACCGT | | |
| | GGCTGTGAAAATACTGAAAAACGAGGCCAATGACCCCGCTCTTAAAGATGAGTTATTAGC | | |
| | AGAAGCAAATGTCATGCAGCAGCTGGACAACCCGTACATCGTGCGGATGATCGGGATATG | | |
| | CGAGGCCGAGTCCTGGATGCTGGTTATGGAGATGGCAGAACTTGGTCCCCTCAATAAGTA | | |
| | TTTGCAGCAGAACAGACATGTCAAGGATAAGAACATCATAGAACTGGTTCATCAGGTTTC | | |
| | CATGGGCATGAAGTACTTGGAGGAGAGCAATTTTGTGCACAGAGATCTGGCTGCAAGAAA | | |
| | TGTGTTGCTAGTTACCCAACATTACGCCAAGATCAGTGATTTCGGACTTTCCAAAGCACTG | | |
| | CGTGCTGATGAAAACTACTACAAGGCCCAGACCCATGGAAAGTGGCCTGTCAAGTGGTAC | | |
| | GCTCCGGAATGCATCAACTACTACAAGTTCTCCAGCAAAAGCGATGTCTGGAGCTTTGGA | | |
| | GTGTTGATGTGGGAAGCATTCTCCTATGGGCAGAAGCCATATCGAGGGATGAAAGGAAGT | | |
| | GAAGTCACCGCTATGTTAGAGAAAGGAGAGCGGATGGGGTGCCCTGCAGGGTGTCCAAG | | |
| | AGAGATGTACGATCTCATGAATCTGTGCTGGACATACGATGTGGAAAACAGGCCCGGATT | | |
| | CGCAGCAGTGGAACTGCGGCTGCGCAATTACTACTATGACGTGGTGAAC | | |
| SEQ ID | | Intracellular activation | PDPAAHLPFFYGSISRAEAEEHLKLAG |
| NO: 046 | | signaling domain | MADGLFLLRQCLRSLGGYVLSLVHDV |
| | | (“ZAP70-ΔKD” in the | RFHHFPIERQLNGTYAIAGGKAHCGPA |
| | | examples) | ELCEFYSRDPDGLPCNLRKPCNRPSGLE |
| | | | PQPGVFDCLRDAMVRDYVRQTWKLE |
| | | | GEALEQAIISQAPQVEKLIATTAHERMP |
| | | | WYHSSLTREEAERKLYSGAQTDGKFLL |
| | | | RPRKEQGTYALSLIYGKTVYHYLISQD |
| | | | KAGKYCIPEGTKFDTLWQLVEYLKLKA |
| | | | DGLIYCLKEACPNSSASNASGAAAPTLP |
| | | | AHPSTLTHPQRRIDTLNSDGYTPEPARIT |
| | | | SPDKPRPMPMDTSVYESPYSDPEELKD |
| | | | KKLFLKRDNL |
| SEQ ID | CCAGACCCCGCGGCGCACCTGCCCTTCTTCTACGGCAGCATCTCGCGTGCCGAGGCCGAG | Intracellular activation | / |
| NO: 047 | GAGCACCTGAAGCTGGCGGGCATGGCGGACGGGCTCTTCCTGCTGCGCCAGTGCCTGCG | signaling domain | |
| | CTCGCTGGGCGGCTATGTGCTGTCGCTCGTGCACGATGTGCGCTTCCACCACTTTCCCATC | (“ZAP70-ΔKD” in the | |
| | GAGCGCCAGCTCAACGGCACCTACGCCATTGCCGGCGGCAAAGCGCACTGTGGACCGGC | examples) | |
| | AGAGCTCTGCGAGTTCTACTCGCGCGACCCCGACGGGCTGCCCTGCAACCTGCGCAAGC | | |
| | CGTGCAACCGGCCGTCGGGCCTCGAGCCGCAGCCGGGGGTCTTCGACTGCCTGCGAGAC | | |
| | GCCATGGTGCGTGACTACGTGCGCCAGACGTGGAAGCTGGAGGGCGAGGCCCTGGAGCA | | |
| | GGCCATCATCAGCCAGGCCCCGCAGGTGGAGAAGCTCATTGCTACGACGGCCCACGAGC | | |
| | GGATGCCCTGGTACCACAGCAGCCTGACGCGTGAGGAGGCCGAGCGCAAACTTTACTCT | | |
| | GGGGCGCAGACCGACGGCAAGTTCCTGCTGAGGCCGCGGAAGGAGCAGGGCACATACG | | |
| | CCCTGTCCCTCATCTATGGGAAGACGGTGTACCACTACCTCATCAGCCAAGACAAGGCGG | | |
| | GCAAGTACTGCATTCCCGAGGGCACCAAGTTTGACACGCTCTGGCAGCTGGTGGAGTATC | | |
| | TGAAGCTGAAGGCGGACGGGCTCATCTACTGCCTGAAGGAGGCCTGCCCCAACAGCAGT | | |
| | GCCAGCAACGCCTCAGGGGCTGCTGCTCCCACACTCCCAGCCCACCCATCCACGTTGACT | | |
| | CATCCTCAGAGACGAATCGACACCCTCAACTCAGATGGATACACCCCTGAGCCAGCACGC | | |
| | ATAACGTCCCCAGACAAACCGCGGCCGATGCCCATGGACACGAGCGTGTATGAGAGCCCC | | |
| | TACAGCGACCCAGAGGAGCTCAAGGACAAGAAGCTCTTCCTGAAGCGCGATAACCTC | | |
| SEQ ID | | Intracellular activation | ASSGMADSANHLPFFFGNITREEAEDY |
| NO: 048 | | signaling domain | LVQGGMSDGLYLLRQSRNYLGGFALSV |
| | | | AHGRKAHHYTIERELNGTYAIAGGRTH |
| | | | ASPADLCHYHSQESDGLVCLLKKPFNR |
| | | | PQGVQPKTGPFEDLKENLIREYVKQTW |
| | | | NLQGQALEQAIISQKPQLEKLIATTAHE |
| | | | KMPWFHGKISREESEQIVLIGSKTNGKF |
| | | | LIRARDNNGSYALCLLHEGKVLHYRID |
| | | | KDKTGKLSIPEGKKFDTLWQLVEHYSY |
| | | | KADGLLRVLTVPCQKIGTQGNVNFGGR |
| | | | PQLPGSHPATWSAGGIISRIKSYSFPKPG |
| | | | HRKSSPAQGNRQESTVSFNPYEPELAP |
| | | | WAADKGPQREALPMDTEVYESPYADP |
| | | | EEIRPKEVYLDRKLL |
| SEQ ID | GCCAGCAGCGGCATGGCTGACAGCGCCAACCACCTGCCCTTCTTTTTCGGCAACATCACC | Intracellular activation | / |
| NO: 049 | CGGGAGGAGGCAGAAGATTACCTGGTCCAGGGGGGCATGAGTGATGGGCTTTATTTGCTG | signaling domain | |
| | CGCCAGAGCCGCAACTACCTGGGTGGCTTCGCCCTGTCCGTGGCCCACGGGAGGAAGGC | | |
| | ACACCACTACACCATCGAGCGGGAGCTGAATGGCACCTACGCCATCGCCGGTGGCAGGA | | |
| | CCCATGCCAGCCCCGCCGACCTCTGCCACTACCACTCCCAGGAGTCTGATGGCCTGGTCT | | |
| | GCCTCCTCAAGAAGCCCTTCAACCGGCCCCAAGGGGTGCAGCCCAAGACTGGGCCCTTT | | |
| | GAGGATTTGAAGGAAAACCTCATCAGGGAATATGTGAAGCAGACATGGAACCTGCAGGG | | |
| | TCAGGCTCTGGAGCAGGCCATCATCAGTCAGAAGCCTCAGCTGGAGAAGCTGATCGCTAC | | |
| | CACAGCCCATGAAAAAATGCCTTGGTTCCATGGAAAAATCTCTCGGGAAGAATCTGAGCA | | |
| | AATTGTCCTGATAGGATCAAAGACAAATGGAAAGTTCCTGATCCGAGCCAGAGACAACAA | | |
| | CGGCTCCTACGCCCTGTGCCTGCTGCACGAAGGGAAGGTGCTGCACTATCGCATCGACAA | | |
| | AGACAAGACAGGGAAGCTCTCCATCCCCGAGGGAAAGAAGTTCGACACGCTCTGGCAGC | | |
| | TAGTCGAGCATTATTCTTATAAAGCAGATGGTTTGTTAAGAGTTCTTACTGTCCCATGTCAA | | |
| | AAAATCGGCACACAGGGAAATGTTAATTTTGGAGGCCGTCCACAACTTCCAGGTTCCCAT | | |
| | CCTGCGACTTGGTCAGCGGGTGGAATAATCTCAAGAATCAAATCATACTCCTTCCCAAAG | | |
| | CCTGGCCACAGAAAGTCCTCCCCTGCCCAAGGGAACCGGCAAGAGAGTACTGTGTCATT | | |
| | CAATCCGTATGAGCCAGAACTTGCACCCTGGGCTGCAGACAAAGGCCCCCAGAGAGAAG | | |
| | CCCTACCCATGGACACAGAGGTGTACGAGAGCCCCTACGCGGACCCCGAGGAGATCAGG | | |
| | CCCAAGGAGGTTTACCTGGACCGAAAGCTGCTG | | |
| SEQ ID | | Intracellular activation | LIADIELGCGNFGSVRQGVYRMRKKQI |
| NO: 050 | | signaling domain | DVAIKVLKQGTEKADTEEMMREAQIM |
| | | | HQLDNPYIVRLIGVCQAEALMLVMEM |
| | | | AGGGPLHKFLVGKREEIPVSNVAELLH |
| | | | QVSMGMKYLEEKNFVHRDLAARNVLL |
| | | | VNRHYAKISDFGLSKALGADDSYYTAR |
| | | | SAGKWPLKWYAPECINFRKFSSRSDVW |
| | | | SYGVTMWEALSYGQKPYKKMKGPEV |
| | | | MAFIEQGKRMECPPECPPELYALMSDC |
| | | | WIYKWEDRPDFLTVEQRMRACYYSL |
| SEQ ID | CTCATAGCTGACATTGAACTTGGCTGCGGCAACTTTGGCTCAGTGCGCCAGGGCGTGTAC | Intracellular activation | / |
| NO: 051 | CGCATGCGCAAGAAGCAGATCGACGTGGCCATCAAGGTGCTGAAGCAGGGCACGGAGAA | signaling domain | |
| | GGCAGACACGGAAGAGATGATGCGCGAGGCGCAGATCATGCACCAGCTGGACAACCCCT | | |
| | ACATCGTGCGGCTCATTGGCGTCTGCCAGGCCGAGGCCCTCATGCTGGTCATGGAGATGG | | |
| | CTGGGGGCGGGCCGCTGCACAAGTTCCTGGTCGGCAAGAGGGAGGAGATCCCTGTGAGC | | |
| | AATGTGGCCGAGCTGCTGCACCAGGTGTCCATGGGGATGAAGTACCTGGAGGAGAAGAA | | |
| | CTTTGTGCACCGTGACCTGGCGGCCCGCAACGTCCTGCTGGTTAACCGGCACTACGCCAA | | |
| | GATCAGCGACTTTGGCCTCTCCAAAGCACTGGGTGCCGACGACAGCTACTACACTGCCCG | | |
| | CTCAGCAGGGAAGTGGCCGCTCAAGTGGTACGCACCCGAATGCATCAACTTCCGCAAGTT | | |
| | CTCCAGCCGCAGCGATGTCTGGAGCTATGGGGTCACCATGTGGGAGGCCTTGTCCTACGG | | |
| | CCAGAAGCCCTACAAGAAGATGAAAGGGCCGGAGGTCATGGCCTTCATCGAGCAGGGCA | | |
| | AGCGGATGGAGTGCCCACCAGAGTGTCCACCCGAACTGTACGCACTCATGAGTGACTGCT | | |
| | GGATCTACAAGTGGGAGGATCGCCCCGACTTCCTGACCGTGGAGCAGCGCATGCGAGCCT | | |
| | GTTACTACAGCCTG | | |
| SEQ ID | | Intracellular activation | TLEDKELGSGNFGTVKKGYYQMKKVV |
| NO: 052 | | signaling domain | KTVAVKILKNEANDPALKDELLAEANV |
| | | | MQQLDNPYIVRMIGICEAESWMLVME |
| | | | MAELGPLNKYLQQNRHVKDKNIIELVH |
| | | | QVSMGMKYLEESNFVHRDLAARNVLL |
| | | | VTQHYAKISDFGLSKALRADENYYKA |
| | | | QTHGKWPVKWYAPECINYYKFSSKSD |
| | | | VWSFGVLMWEAFSYGQKPYRGMKGS |
| | | | EVTAMLEKGERMGCPAGCPREMYDLM |
| | | | NLCWTYDVENRPGFAAVELRLRNYYY |
| SEQ ID | ACGCTGGAAGACAAAGAACTGGGCTCTGGTAATTTTGGAACTGTGAAAAAGGGCTACTA | Intracellular activation | / |
| NO: 053 | CCAAATGAAAAAAGTTGTGAAAACCGTGGCTGTGAAAATACTGAAAAACGAGGCCAATG | signaling domain | |
| | ACCCCGCTCTTAAAGATGAGTTATTAGCAGAAGCAAATGTCATGCAGCAGCTGGACAACC | | |
| | CGTACATCGTGCGGATGATCGGGATATGCGAGGCCGAGTCCTGGATGCTGGTTATGGAGAT | | |
| | GGCAGAACTTGGTCCCCTCAATAAGTATTTGCAGCAGAACAGACATGTCAAGGATAAGAA | | |
| | CATCATAGAACTGGTTCATCAGGTTTCCATGGGCATGAAGTACTTGGAGGAGAGCAATTTT | | |
| | GTGCACAGAGATCTGGCTGCAAGAAATGTGTTGCTAGTTACCCAACATTACGCCAAGATC | | |
| | AGTGATTTCGGACTTTCCAAAGCACTGCGTGCTGATGAAAACTACTACAAGGCCCAGACC | | |
| | CATGGAAAGTGGCCTGTCAAGTGGTACGCTCCGGAATGCATCAACTACTACAAGTTCTCC | | |
| | AGCAAAAGCGATGTCTGGAGCTTTGGAGTGTTGATGTGGGAAGCATTCTCCTATGGGCAG | | |
| | AAGCCATATCGAGGGATGAAAGGAAGTGAAGTCACCGCTATGTTAGAGAAAGGAGAGCG | | |
| | GATGGGGTGCCCTGCAGGGTGTCCAAGAGAGATGTACGATCTCATGAATCTGTGCTGGAC | | |
| | ATACGATGTGGAAAACAGGCCCGGATTCGCAGCAGTGGAACTGCGGCTGCGCAATTACTA | | |
| | CTAT | | |
| SEQ ID | | Intracellular spacer | CSRAARGTIGARRTGQPLKEDPSAVPVF |
| NO: 054 | | domain (“Intracellular | S |
| | | spacer domain in | |
| | | Truncated PD-1” in | |
| | | the Examples) | |
| SEQ ID | TGCTCCCGGGCCGCACGAGGGACAATAGGAGCCAGGCGCACCGGCCAGCCCCTGAAGGA | Intracellular spacer | / |
| NO: 055 | GGACCCCTCAGCCGTGCCTGTGTTCTCT | domain (“Intracellular | |
| | | spacer domain in | |
| | | Truncated PD-1” in | |
| | | the Examples) | |
| SEQ ID | | Intracellular spacer | CSRAARGTIGARRTGQPLKEDPSAVPVF |
| NO: 056 | | domain (“Intracellular | SVDYGELDFQWREKTPEPPVPCVPEQT |
| | | spacer domain in Full- | EYATIVFPSGMGTSSPARRGSADGPRSA |
| | | length PD-1” in the | QPLRPEDGHCSWPL |
| | | Examples) | |
| SEQ ID | TGCTCCCGGGCCGCACGAGGGACAATAGGAGCCAGGCGCACCGGCCAGCCCCTGAAGGA | Intracellular spacer | / |
| NO: 057 | GGACCCCTCAGCCGTGCCTGTGTTCTCTGTGGACTATGGGGAGCTGGATTTCCAGTGGCG | domain (“Intracellular | |
| | AGAGAAGACCCCGGAGCCCCCCGTGCCCTGTGTCCCTGAGCAGACGGAGTATGCCACCA | spacer domain in Full- | |
| | TTGTCTTTCCTAGCGGAATGGGCACCTCATCCCCCGCCCGCAGGGGCTCAGCTGACGGCC | length PD-1” in the | |
| | CTCGGAGTGCCCAGCCACTGAGGCCTGAGGATGGACACTGCTCTTGGCCCCTC | Examples) | |
| SEQ ID | | Intracellular linker | GGSGGTGGSGGT |
| NO: 058 | | domain (“SL” in the | |
| | | examples) | |
| SEQ ID | GGCGGGTCTGGCGGGACAGGAGGTTCAGGTGGCACA | Intracellular linker | / |
| NO: 059 | | domain (“SL” in the | |
| | | examples) | |
| SEQ ID | | Intracellular linker | GSTSGSGKPGSGEGSTKGSTSGSGKPGS |
| NO: 060 | | domain (“ML” in the | GEGSTK |
| | | examples) | |
| SEQ ID | GGTTCAACTTCTGGCTCTGGGAAACCAGGAAGCGGCGAAGGGTCCACCAAGGGAAGCA | Intracellular linker | / |
| NO: 061 | CCAGTGGTTCAGGTAAGCCTGGTTCTGGTGAAGGTAGCACTAAA | domain (“ML” in the | |
| | | examples) | |
| SEQ ID | | Intracellular linker | SAGGSAGGSAGGSAGGSAGGSGSAGG |
| NO: 062 | | domain (“LL1” in the | SAGGSTSAGGSAGGSAGGSAGGSAGG |
| | | examples) | SGSAGGSAGGSTSAGGSAGGSAGGSA |
| | | | GGSAGGSGSAGGSAGGSTSAGGSAGG |
| | | | SAGGSAGGSAGGGGSGGTGGSGGT |
| SEQ ID | AGCGCAGGCGGATCAGCTGGAGGGTCTGCAGGGGGTAGTGCAGGTGGCTCAGCTGGCGG | Intracellular linker | / |
| NO: 063 | GAGCGGCTCAGCTGGGGGATCTGCTGGTGGCAGTACCTCAGCAGGCGGTAGCGCCGGAG | domain (“LLI” in the | |
| | GTTCTGCTGGTGGCTCCGCAGGAGGGTCTGCAGGCGGTTCCGGGAGTGCAGGTGGATCT | examples) | |
| | GCAGGTGGGTCAACAAGTGCTGGTGGATCCGCAGGAGGTTCAGCAGGCGGGAGTGCTGG | | |
| | AGGCTCTGCAGGCGGTAGCGGGAGTGCCGGTGGCAGCGCAGGGGGAAGCACTAGTGCT | | |
| | GGAGGCAGTGCAGGTGGCAGCGCAGGAGGCTCTGCCGGGGGAAGCGCCGGGGGCggcgG | | |
| | GTCTGGCGGGACAGGAGGTTCAGGTGGCACA | | |
| SEQ ID | | Intracellular linker | SAGGSAGGSAGGSAGGSAGGSGSAGG |
| NO: 064 | | domain (“LL2” in the | SAGGSTSAGGSAGGSAGGSAGGSAGG |
| | | examples) | SGSAGGSAGGSTSAGGSAGGSAGGSA |
| | | | GGSAGGSGSAGGSAGGSTSAGGSAGG |
| | | | SAGGSAGGSAGG |
| SEQ ID | AGCGCAGGCGGATCAGCTGGAGGGTCTGCAGGGGGTAGTGCAGGTGGCTCAGCTGGCGG | Intracellular linker | / |
| NO: 065 | GAGCGGCTCAGCTGGGGGATCTGCTGGTGGCAGTACCTCAGCAGGCGGTAGCGCCGGAG | domain (“LL2” in the | |
| | GTTCTGCTGGTGGCTCCGCAGGAGGGTCTGCAGGCGGTTCCGGGAGTGCAGGTGGATCT | examples) | |
| | GCAGGTGGGTCAACAAGTGCTGGTGGATCCGCAGGAGGTTCAGCAGGCGGGAGTGCTGG | | |
| | AGGCTCTGCAGGCGGTAGCGGGAGTGCCGGTGGCAGCGCAGGGGGAAGCACTAGTGCT | | |
| | GGAGGCAGTGCAGGTGGCAGCGCAGGAGGCTCTGCCGGGGGAAGCGCCGGGGGC | | |
| SEQ ID | | Intracellular linker | GGSGGT |
| NO: 066 | | domain | |

According to another aspect of the present application, there is provided a nucleic acid molecule encoding any chimeric antigen receptor described above.

Optionally, the nucleic acid molecule comprises an extracellular target molecule binding domain nucleic acid fragment, a transmembrane domain nucleic acid fragment, an intracellular activation signaling domain nucleic acid fragment, an extracellular spacer domain nucleic acid fragment, an intracellular detection signaling nucleic acid fragment, an intracellular spacer domain nucleic acid fragment, and an intracellular linker domain fragment.

Optionally, the extracellular target molecule binding domain nucleic acid fragment comprises at least one of a nucleic acid sequence comprising SEQ ID NO:002, a nucleic acid sequence comprising SEQ ID NO:004, a nucleic acid sequence comprising SEQ ID NO:006, a nucleic acid sequence comprising SEQ ID NO:008 and a nucleic acid sequence comprising SEQ ID NO:010.

Optionally, the transmembrane domain nucleic acid fragment comprises at least one of a nucleic acid sequence comprising SEQ ID NO:013 and a nucleic acid sequence comprising SEQ ID NO:015.

Optionally, the intracellular activation signaling domain nucleic acid fragment comprises at least one of a nucleic acid sequence comprising SEQ ID NO:043, a nucleic acid sequence comprising SEQ ID NO:045, a nucleic acid sequence comprising SEQ ID NO:047, a nucleic acid sequence comprising SEQ ID NO:049, a nucleic acid sequence comprising SEQ ID NO:051 and a nucleic acid sequence comprising SEQ ID NO:053.

Optionally, the extracellular spacer domain nucleic acid fragment comprises at least one of a nucleic acid sequence comprising SEQ ID NO:017 and a nucleic acid sequence comprising SEQ ID NO:019.

Optionally, the intracellular detection signaling domain nucleic acid fragment comprises at least one of a nucleic acid sequence comprising SEQ ID NO:021, a nucleic acid sequence comprising SEQ ID NO:023, a nucleic acid sequence comprising SEQ ID NO:025, a nucleic acid sequence comprising SEQ ID NO:027, a nucleic acid sequence comprising SEQ ID NO:029, a nucleic acid sequence comprising SEQ ID NO:031, a nucleic acid sequence comprising SEQ ID NO:033, a nucleic acid sequence comprising SEQ ID NO:035, a nucleic acid sequence comprising SEQ ID NO:037, a nucleic acid sequence comprising SEQ ID NO:039 and a nucleic acid sequence comprising SEQ ID NO:041.

Optionally, the intracellular spacer domain nucleic acid fragment comprises at least one of a nucleic acid sequence comprising SEQ ID NO:055 and a nucleic acid sequence comprising SEQ ID NO:057.

Optionally, the intracellular linker domain fragment comprises at least one of a nucleic acid sequence comprising SEQ ID NO:059, a nucleic acid sequence comprising SEQ ID NO:061, a nucleic acid sequence comprising SEQ ID NO:063 and a nucleic acid sequence comprising SEQ ID NO: 065.

With respect to the sequences in the present application including amino acid sequence and nucleic acid sequence, homologous sequences are all within the protection scope of the present application.

Sequence Homology: the term “sequence homology” used in the present application is defined as the significant similarity in terms of coding sequence among two or more nucleic acid molecules or among two or more protein sequences, for example having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or at least 100% sequence coding identity.

According to another aspect of the present application, there is provided a vector comprising the above-mentioned nucleic acid molecule.

Optionally, the vector is at least one of a viral vector, a modified mRNA vector and a transposon-mediated gene transfer vector.

According to another aspect of the present application, there is provided a host cell comprising any chimeric antigen receptor described above, any nucleic acid molecule described above, or any vector described above.

According to another aspect of the present application, there is provided a host cell population comprising at least one of the above-mentioned host cells.

According to another aspect of the present application, there is provided a pharmaceutical composition comprising at least one of any antigen chimeric receptor described above, any nucleic acid molecule described above, any nucleic acid molecule described above, any vector described above, any host cell described above, and any host cell population described above.

Optionally, the pharmaceutical composition further comprises cytokine.

Optionally, the cytokine is at least one of interferon-γ and interleukin.

Optionally, the pharmaceutical composition further comprises monoclonal antibody.

Optionally, the monoclonal antibody is at least one of cetuximab, alemtuzumab, ipilimumab and ofatumumab.

According to another aspect of the present application, there is also provided a method for using any pharmaceutical composition mentioned above, which comprises following steps:
1) obtaining an human immune cell;
2) modifying the human immune cell to obtain modified immune cell;
wherein, the modified immune cell comprise at least one of any chimeric antigen receptor, any nucleic acid molecule described above, any vector described above, any host cell described above and any host cell population described above; and
3) infusing the modified immune cell back to the human body.

Optionally, step 3) further comprises following steps:
3-1) applying at least one of cytokine and monoclonal antibody to the whole or part of the human body; and
3-2) infusing the modified immune cell back to the human body.

According to another aspect of the present application, there is provided use of at least one of any antigen chimeric receptor described above, any nucleic acid molecule described above, any vector described above, any host cell described above, any host cell population described above and any pharmaceutical composition described above in the preparation of a drug for treating PD-L1-positive tumor or tumor that up-regulates PD-L1 expression levels in response to interferon-γ.

According to another aspect of the present application, there is provided use of at least one of any antigen chimeric receptor described above, any nucleic acid molecule described above, any vector described above, any host cell described above, any host cell population described above and any pharmaceutical composition described above in the treatment of PD-L1-positive tumor or tumor that up-regulates PD-L1 expression levels in response to interferon-γ.

According to another aspect of the present application, there is provided use of at least one of any antigen chimeric receptor described above, any nucleic acid molecule described above, any vector described above, any host cell described above, any host cell population described above and any pharmaceutical composition described above in the preparation of a drug for treating solid tumor and/or blood cancer.

According to another aspect of the present application, there is provided use of at least one of any antigen chimeric receptor described above, any nucleic acid molecule described above, any vector described above, any host cell described above, any host cell population described above and any pharmaceutical composition described above in the preparation of a drug for treating following tumors: examples of various cancers include, but are not limited to, breast cancer, rectal cancer, skin cancer, colon cancer, pancreatic cancer, liver cancer, ovarian cancer, prostate cancer, brain cancer, kidney cancer, lung cancer, lymphoma and melanoma.

According to another aspect of the present application, there is provided use of at least one of any antigen chimeric receptor described above, any nucleic acid molecule described above, any vector described above, any host cell described above, any host cell population described above and any pharmaceutical composition described above in the treatment of solid tumor and/or blood cancer.

According to another aspect of the present application, there is provided use of at least one of any antigen chimeric receptor described above, any nucleic acid molecule described above, any vector described above, any host cell described above, any host cell population described above and any pharmaceutical composition described above in the treatment of following tumors: examples of various cancers include, but are not limited to, breast cancer, rectal cancer, skin cancer, colon cancer, pancreatic cancer, liver cancer, ovarian cancer, prostate cancer, brain cancer, kidney cancer, lung cancer, lymphoma and melanoma.

According to another aspect of the present application, there is provided use of at least one of any antigen chimeric receptor described above, any nucleic acid molecule described above, any vector described above, any host cell described above, any host cell population described above and any pharmaceutical composition described above in the preparation of a drug for treating following diseases: infection, inflammatory disease, immune disease and neurological disease.

According to another aspect of the present application, there is provided use of at least one of any antigen chimeric receptor described above, any nucleic acid molecule described above, any vector described above, any host cell described above, any host cell population described above and any pharmaceutical composition described above in the treatment of following diseases: infection, inflammatory disease, immune disease and neurological disease.

According to another aspect of the present application, there is provided a method for treating tumor comprising:
1) obtaining an human immune cell;
2) modifying the human immune cell to obtain modified immune cell;
wherein the modified immune cell comprises at least one of any chimeric antigen receptor described above, any nucleic acid molecule described above, any vector described above, any host cell described above and any host cell population mentioned above; and
3) infusing the modified immune cell back to the human body.

Optionally, step 3) further comprises following steps:
3-1) applying at least one of cytokine and monoclonal antibody to the whole or part of the human body; and
3-2) infusing the modified immune cell back to the human body.

According to another aspect of the present application, there is provided a method for treating a disease, which comprises following steps:
1) obtaining an human immune cell;
2) modifying the human immune cell to obtain modified immune cell;
wherein the modified immune cell comprises at least one of any chimeric antigen receptor described above, any nucleic acid molecule described above, any vector described above, any host cell described above and any host cell population mentioned above; and
3) infusing the modified immune cell back to the human body;
wherein, the disease comprises infection, inflammatory disease, immune disease, neurological disease.

Optionally, step 3) further comprises following steps:
3-1) applying at least one of cytokine and monoclonal antibody to the whole or part of the human body; and
3-2) infusing the modified immune cell back to the human body.

The beneficial effects that the present application can achieve comprise:
1) With respect to the chimeric antigen receptor provided in the present application, the design of its intracellular signaling domain enhances the activation of host immune cell and the killing effect aganist cancer cell, and expands the adaptability of chimeric antigen receptor to the modification of a variety of immune cells.
2) The chimeric antigen receptor provided in the present application is preferably based on the modification of the PD-1/PD-L1 signaling pathway of the immune checkpoint, and re-encodes and modifies a variety of cells (such as immune T cell or phagocyte and the like) to better identify and kill specific cancer cells. When the cancer cells expressing the immune checkpoint inhibitory signal PD-L1 as PD-1 molecule ligand try to inhibit the function of immune cells (such as immune T cells or phagocytes and the like) by means of brake blocking mechanism in the immune checkpoint PD-1/PD-L1 signaling pathway, the immune cells (such as immune T cells or phagocytes and the like) modified by re-encoding with this new generation of immune checkpoint PD-1-based chimeric antigen receptor molecular machine will not be inhibited by cancer cells, but will be further activated, generating specific immune response against the corresponding cancer cells, thereby identifying and killing the corresponding cancer cells.
3) The chimeric antigen receptor provided in the present application can better identify and kill specific cancer cells, including breast cancer, rectal cancer, skin cancer, colon cancer, pancreatic cancer, liver cancer, ovarian cancer, prostate cancer, brain cancer, kidney cancer, lung cancer, lymphoma and melanoma and the like.

### BRIEF DESCRIPTION OF THE DRAWINGS

FIG. 1A shows a schematic diagram of the construction of the chimeric antigen receptor artificial molecular machine based on an extracellular target molecule binding domain (such as PD-1 extracellular fragment or targeting scFv), an extracellular spacer domain, a transmembrane domain and intracellular signaling domain according to the present application.

FIG. 1B shows a schematic diagram of the construction of the chimeric antigen receptor artificial molecular machine based on an extracellular target molecule binding domain (such as PD-1 extracellular fragment or targeting scFv), an extracellular spacer domain, a transmembrane domain and an intracellular activation signaling domain belonging to the activation module according to the present application.

FIG. 1C shows a schematic diagram of the construction of the chimeric antigen receptor artificial molecular machine based on an extracellular target molecule binding domain (such as PD-1 extracellular fragment or targeting scFv), an extracellular spacer domain, a transmembrane domain, an intracellular detection signaling domain belonging to the detection module and an intracellular activation signaling domain belonging to the activation module according to the present application.

FIG. 1D shows a schematic diagram of the construction of the chimeric antigen receptor artificial molecular machine based on an extracellular target molecule binding domain (such as PD-1 extracellular fragment or targeting scFv), an extracellular spacer domain, a transmembrane domain, an intracellular detection signaling domain belonging to the detection module, an intracellular linker domain and an intracellular activation signaling domain belonging to the activation module according to the present application.

FIG. 1E shows a schematic diagram of the construction of the chimeric antigen receptor artificial molecular machine based on an extracellular target molecule binding domain (such as PD-1 extracellular fragment or targeting scFv), an extracellular spacer domain, a transmembrane domain, an intracellular spacer domain and an intracellular signaling domain according to the present application.

FIG. 1F shows a schematic diagram of the construction of the chimeric antigen receptor artificial molecular machine based on an extracellular target molecule binding domain (such as PD-1 extracellular fragment or targeting scFv), an extracellular spacer domain, a transmembrane domain, an intracellular spacer domain and an intracellular activation signaling domain belonging to the activation module according to the present application.

FIG. 1G shows a schematic diagram of the construction of the chimeric antigen receptor artificial molecular machine based on an extracellular target molecule binding domain (such as PD-1 extracellular fragment or targeting scFv), an extracellular spacer domain, a transmembrane domain, an intracellular spacer domain, an intracellular detection signaling domain belonging to the detection module and an intracellular activation signaling domain belonging to the activation module according to the present application.

FIG. 1H shows a schematic diagram of the construction of the chimeric antigen receptor artificial molecular machine based on an extracellular target molecule binding domain (such as PD-1 extracellular fragment or targeting scFv), an extracellular spacer domain, a transmembrane domain, an intracellular spacer domain, an intracellular detection signaling domain belonging to detection module, an intracellular linker domain and an intracellular activation signaling domain (belonging to activation module) according to the present application.

FIGS. 2A-2B show a schematic diagram of the signal activation of a chimeric antigen receptor artificial molecular machine comprising an extracellular target molecule binding domain, wherein FIG. 2A is a schematic diagram of the signal activation of the artificial molecular machine under the condition that the tyrosine kinase activation signal is input, FIG. 2B is a schematic diagram of signal activation of a chimeric antigen receptor artificial molecular machine comprising an extracellular target molecule binding domain (such as the extracellular portion of PD-1) under the condition that target molecule recognition binding signal (such as PD-L1) is input.

FIGS. 3A-3D show a comparison of an endogenous immune cell and an immune cell modified with the chimeric antigen receptor according to the present disclosure. wherein, FIG. 3A shows the performance of the endogenous natural lymphocyte facing the cancer cell, FIG. 3B shows the performance of the lymphocyte modified with the chimeric antigen receptor according to the present disclosure facing the cancer cell, and the gray scale of the lymphocyte corresponds to the tumor-killing ability thereof. FIG. 3C shows the performance of endogenous natural phagocyte facing the cancer cell, FIG. 3D shows the performance of the phagocyte modified with the chimeric antigen receptors according to the present disclosure facing cancer cell, phagocytosing, and the gray scale of phagocyte corresponds to the tumor-killing ability thereof.

FIGS. 4A-4B show an exemplary method of administering the chimeric antigen receptor according to the present disclosure by different types of immune cells. wherein, FIG. 4A shows an exemplary method of administering the chimeric antigen receptor according to the present disclosure by lymphocytes; and FIG. 4B shows an exemplary method of administering the chimeric antigen receptors of the present disclosure by phagocytes.

FIG. 5 shows performance result histogram of different artificial molecular machines in the state of purified protein (the data therein is shown as mean±standard deviation, C #9(+) group n=3, C #10(+) group n=3) under the condition that the Src family protein non-receptor protein tyrosine kinase Lck (Lymphocyte-specific protein tyrosine kinase, lymphocyte-specific protein tyrosine kinase) provides the activation protein tyrosine phosphorylation signal, wherein, the imaging reading index represents the degree of the response ability of the artificial molecular machine to the stimulus signal after quantification of data and the degree of the release and activation of the self-activating element of the artificial molecular machine based on the molecular conformational change, which is simultaneously triggered in response to the stimulus signal. Here, the non-receptor-type protein tyrosine kinase Lck can promote the activation of protein tyrosine phosphorylation signal and play a role in providing input of specific protein tyrosine phosphorylation signal.

FIG. 6A shows performance result histogram of different artificial molecular machines in human HeLa cell (the data therein is shown as mean±standard deviation, for each of C #9 group to C #16, n=5) under the condition that the sodium pervanadate as tyrosine phosphatase inhibitor activates the protein tyrosine phosphorylation signal, wherein, the imaging reading index represents the degree of the response ability of the artificial molecular machine to the stimulus signal after quantification of data and the degree of the release and activation of the self-activating element of the artificial molecular machine based on the molecular conformational change, which is simultaneously triggered in response to the stimulus signal. Here, the sodium pervanadate as tyrosine phosphatase inhibitor can inhibit the dephosphorylation of intracellular protein, thereby promoting the activation of protein tyrosine phosphorylation signal and playing a role in providing input of protein tyrosine phosphorylation signal.

FIG. 6B shows performance result histogram of different artificial molecular machines in human HeLa cell (the data therein is shown as mean±standard deviation, for each of C #9-A group and C #15-A group, n=5; for each of C #9-B group and C #15-B group, n=3) under the condition A where the sodium pervanadate as tyrosine phosphatase inhibitor activates the protein tyrosine phosphorylation signal or the condition B where the epidermal growth factor (EGF) activates the signal, wherein, the imaging reading index represents the degree of the response ability of the artificial molecular machine to the stimulus signal after quantification of data and the degree of the release and activation of the self-activating element of the artificial molecular machine based on the molecular conformational change, which is simultaneously triggered in response to the stimulus signal.

FIG. 6C shows performance result histogram of different artificial molecular machines in mouse embryonic fibroblast (MEF) (for each of C #9-A group, C #9-B group, C #15-A group and C #15-B group, n=5) under the condition A where the sodium pervanadate as tyrosine phosphatase inhibitor activates the protein tyrosine phosphorylation signal or the condition B where the platelet-derived growth factor (PDGF) activates the signal, wherein, the imaging reading index represents the degree of the response ability of the artificial molecular machine to the stimulus signal after quantification of data and the degree of the release and activation of the self-activating element of the artificial molecular machine based on the molecular conformational change, which is simultaneously triggered in response to the stimulus signal.

FIG. 7A shows the expression distribution of different immune checkpoint PD-1 fusion-based chimeric antigen receptor artificial molecular machines in human HeLa cell and detection result for the ability to respond to protein tyrosine phosphorylation signal stimulated by the sodium pervanadate as the tyrosine phosphatase inhibitor; wherein, the experimental group refers to human HeLa cell modified with the immune checkpoint PD-1 fusion-based chimeric antigen receptor C #17 version according to the present disclosure while the control group refers to Human HeLa cell modified with the immune checkpoint PD-1 fusion-based chimeric antigen receptor C #18 version according to the present disclosure, the color bar heat map under the Figures from left to right represents from low to high response ability of the chimeric antigen receptor to stimulus signal, and from low to high degree of the release and activation of the self-activating element of the chimeric antigen receptor based on the molecular conformational change, which is simultaneously triggered in response to the stimulus signal. Here, the sodium pervanadate as tyrosine phosphatase inhibitor can inhibit the dephosphorylation of intracellular protein, thereby promoting the activation of protein tyrosine phosphorylation signal and playing a role in providing input of protein tyrosine phosphorylation signal.

FIG. 7B shows the expression distribution of different immune checkpoint PD-1 fusion-based chimeric antigen receptor artificial molecular machines in human HeLa cell and detection result for the ability to respond to protein tyrosine phosphorylation signal stimulated by the sodium pervanadate as the tyrosine phosphatase inhibitor; wherein, the experimental group refers to human HeLa cell modified with the immune checkpoint PD-1 fusion-based chimeric antigen receptor C #19 version according to the present disclosure while the control group refers to human HeLa cell modified with the immune checkpoint PD-1 fusion-based chimeric antigen receptor C #20 version according to the present disclosure, the color bar heat map under the Figures from left to right represents from low to high response ability of the chimeric antigen receptor to stimulus signal, and from low to high degree of the release and activation of the self-activating element of the chimeric antigen receptor based on the molecular conformational change, which is simultaneously triggered in response to the stimulus signal. Here, the sodium pervanadate as tyrosine phosphatase inhibitor can inhibit the dephosphorylation of intracellular protein, thereby promoting the activation of protein tyrosine phosphorylation signal and playing a role in providing input of protein tyrosine phosphorylation signal.

FIG. 7C shows performance result histogram of different immune checkpoint PD-1 fusion-based chimeric antigen receptor artificial molecular machines in human HeLa cell (the data therein is shown as mean±standard deviation, for each of C #17 group to C #20 group, n=10) under the condition that the sodium pervanadate as tyrosine phosphatase inhibitor activates the protein tyrosine phosphorylation signal, wherein, the imaging reading index represents the degree of the response ability of chimeric antigen receptor to the stimulus signal after quantification of data and the degree of the release and activation of the self-activating element of the chimeric antigen receptor based on the molecular conformational change, which is simultaneously triggered in response to the stimulus signal.

FIG. 8A shows the expression distribution of different immune checkpoint PD-1 fusion-based chimeric antigen receptor artificial molecular machines in human Jurkat E6-1 cell and detection result for the ability to respond to protein tyrosine phosphorylation signal stimulated by the sodium pervanadate as the tyrosine phosphatase inhibitor; wherein, the experimental group refers to human Jurkat E6-1 cell modified with the immune checkpoint PD-1 fusion-based chimeric antigen receptor C #19 version according to the present disclosure while the control group refers to human Jurkat E6-1 cell modified with the immune checkpoint PD-1 fusion-based chimeric antigen receptor C #20 version according to the present disclosure, the color bar heat map under the Figures from left to right represents from low to high response ability of the chimeric antigen receptor to stimulus signal, and from low to high degree of the release and activation of the self-activating element of the chimeric antigen receptor based on the molecular conformational change, which is simultaneously triggered in response to the stimulus signal. Here, the sodium pervanadate as tyrosine phosphatase inhibitor can inhibit the dephosphorylation of intracellular protein, thereby promoting the activation of protein tyrosine phosphorylation signal and playing a role in providing input of protein tyrosine phosphorylation signal.

FIG. 8B shows performance result histogram of different immune checkpoint PD-1 fusion-based chimeric antigen receptor artificial molecular machines in human Jurkat E6-1 cell (the data therein is shown as mean±standard deviation, for each of C #19 group and C #20 group, n=10) under the condition that the sodium pervanadate as tyrosine phosphatase inhibitor activates the protein tyrosine phosphorylation signal, wherein, the imaging reading index represents the degree of the response ability of chimeric antigen receptor to the stimulus signal after quantification of data and the degree of the release and activation of the self-activating element of the chimeric antigen receptor based on the molecular conformational change, which is simultaneously triggered in response to the stimulus signal.

FIG. 9A shows the expression distribution of different immune checkpoint PD-1 fusion-based chimeric antigen receptor artificial molecular machines in human HeLa cell and detection result in response to human PD-L1 signal stimulated by the human PD-L1-modified microspheres; wherein, the experimental group refers to human HeLa cell modified with the immune checkpoint PD-1 fusion-based chimeric antigen receptor C #19 version according to the present disclosure while the control group refers to human HeLa cell modified with the immune checkpoint PD-1 fusion-based chimeric antigen receptor C #20 version according to the present disclosure, the color bar heat map on the right of Figures from bottom to up represents from low to high response ability of the chimeric antigen receptor to stimulus signal, and from low to high degree of the release and activation of the self-activating element of the chimeric antigen receptor based on the molecular conformational change, which is simultaneously triggered in response to the stimulus signal; and the phase-contrast imaging experimental figures provide the image information of the interaction between cell and microsphere. Here, the human PD-L1-modified microspheres play a role of providing human PD-L1 signal input.

FIG. 9B shows the expression distribution of different immune checkpoint PD-1 fusion-based chimeric antigen receptor artificial molecular machines in human Jurkat E6-1 cell and detection result in response to human PD-L1 signal stimulated by the human PD-L1-modified microspheres; wherein, the experimental group refers to human Jurkat E6-1 cell modified with the immune checkpoint PD-1 fusion-based chimeric antigen receptor C #19 version according to the present disclosure while the control group refers to human Jurkat E6-1 cell modified with the immune checkpoint PD-1 fusion-based chimeric antigen receptor C #20 version according to the present disclosure, the color bar heat map on the right of Figures from bottom to up represents from low to high response ability of the chimeric antigen receptor to stimulus signal, and from low to high degree of the release and activation of the self-activating element of the chimeric antigen receptor based on the molecular conformational change, which is simultaneously triggered in response to the stimulus signal; and the phase-contrast imaging experimental figures provide the image information of the interaction between cell and microsphere. Here, the human PD-L1-modified microspheres play a role of providing human PD-L1 signal input.

FIG. 9C shows performance result histogram of different immune checkpoint PD-1 fusion-based chimeric antigen receptor artificial molecular machines in human HeLa cell (the data therein is shown as mean±standard deviation, for each of C #17 group to C #20 group, n=10) under the condition that the signal is stimulated by the human PD-L1-modified microsphere, wherein, the imaging reading index represents the degree of the response ability of chimeric antigen receptor to the stimulus signal after quantification of data and the degree of the release and activation of the self-activating element of the chimeric antigen receptor based on the molecular conformational change, which is simultaneously triggered in response to the stimulus signal.

FIG. 9D shows performance result histogram of different immune checkpoint PD-1 fusion-based chimeric antigen receptor artificial molecular machines in human Jurkat E6-1 cell (the data therein is shown as mean±standard deviation, for each of C #19 group and C #20 group, n=10) under the condition that the signal is stimulated by the human PD-L1-modified microsphere, wherein, the imaging reading index represents the degree of the response ability of chimeric antigen receptor to the stimulus signal after quantification of data and the degree of the release and activation of the self-activating element of the chimeric antigen receptor based on the molecular conformational change, which is simultaneously triggered in response to the stimulus signal.

FIG. 10 shows histogram of different T cell activation level performances under the condition that Jurkat E6-1 cell modified with the immune checkpoint PD-1 fusion-based chimeric antigen receptor artificial molecular machine and PD-L1 high-expressing human breast cancer cell MDA-MB-231 pretreated with interferon-γ are co-cultured (the data therein is shown as mean±standard deviation, for C #19 (+) group, n=4, for other groups, n=6, (+) represents the condition where the Jurket E6-1 cell is co-cultured with human breast cancer cell pretreated with interferon-γ, (−) represents the condition where the Jurket E6-1 cell is cultured alone, and the T cell activation read index represents relative expression level of the activating molecule CD69 on the surface of T lymphocyte.

FIG. 11 shows histogram of different T cell activation level performances under the condition that Jurkat E6-1 cell modified with the immune checkpoint PD-1 fusion-based chimeric antigen receptor artificial molecular machine comprising different lengths of intracellular linker domains and PD-L1 high-expressing human breast cancer cell MDA-MB-231 pretreated with interferon-γ are co-cultured (the data therein is shown as mean±standard deviation, for C #19 (+) group, n=4, for C #19 (−) group, n=6, for the average value of other groups, n=1, (+) represents the condition where the Jurket E6-1 cell is co-cultured with human breast cancer cell pretreated with interferon-γ, (−) represents the condition where the Jurket E6-1 cell is cultured alone, and the T cell activation read index represents relative expression level of the activating molecule CD69 on the surface of T lymphocyte.

FIG. 12 shows the expression level of different immune checkpoint PD-1 fusion-based chimeric antigen receptors in human immunogenic primary T cell (data therein is shown as geometric mean, for all geometric mean, n=1). Please refer to FIG. 28 and related description according to the present application for the information of each component included in the immune checkpoint PD-1 fusion-based chimeric antigen receptor C #1 version, C #2 version, C #3 version, C #4 version and C #5 version.

FIG. 13A shows the establishment and analysis test flow of the cytotoxicity experimental model of the vitro co-culture of T cell and PD-L1 positive-human rectal cancer cell involved in the present application.

FIG. 13B shows the quantitative analysis result of the cytotoxic effect of the in vitro co-culture of human immunogenic primary T cell and PD-L1 positive human colorectal cancer cell DLD1 cell-modified strain in the presence of PD-1 immune checkpoint inhibitor (data therein is shown as mean±standard deviation, for all, n=3); wherein, the human immunogenic primary T cell in the control group is the one that has not been modified by the chimeric antigen receptor artificial molecular machine, the target cell survival index represents relative cell number of human colorectal cancer cells expressing the reporter gene firefly luciferase in the cell culture system, and PD-1 immune checkpoint inhibitor is nivolumab or pembrolizumab.

FIG. 13C shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human colorectal cancer cell DLD1 cell-modified strain (data therein is shown as mean±standard deviation, for all, n=3). Please refer to FIG. 28 and related description according to the present application for the information of each component included in the immune checkpoint PD-1 fusion-based chimeric antigen receptor C #1 version, C #2 version, C #3 version, C #4 version and C #5 version; wherein, the human immunogenic primary T cell in the control group is the one that has not been modified by the chimeric antigen receptor artificial molecular machine, and the target cell survival index represents relative cell number of human colorectal cancer cells expressing the reporter gene firefly luciferase in the cell culture system.

FIG. 14A shows the establishment and analysis test flow of the cytotoxicity experimental model of the vitro co-culture of T cell and PD-L1 positive human breast cancer cell involved in the present application.

FIG. 14B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human breast cancer cell MDA-MB-231 (data therein is shown as mean±standard deviation, for all, n=3). Please refer to FIG. 28 and related description according to the present application for the information of each component included in the immune checkpoint PD-1 fusion-based chimeric antigen receptor C #2 version, C #3 version and C #5 version; wherein, the target cell survival index represents relative cell number of human breast cancer cells expressing the reporter gene firefly luciferase in the cell culture system.

FIG. 15A shows the establishment and analysis test flow of the cytotoxicity experimental model of the vitro co-culture of T cell and PD-L1 positive human breast cancer cell involved in the present application.

FIG. 15B shows the quantitative analysis result of the cytotoxic effect of the in vitro co-culture of human immunogenic primary T cell and PD-L1 positive human breast cancer cell MDA-MB-231 in the presence of PD-1 immune checkpoint inhibitor (data therein is shown as mean±standard deviation, for all, n=3). wherein, the human immunogenic primary T cell in the control group is the one that has not been modified by the chimeric antigen receptor artificial molecular machine, the target cell survival index represents relative cell number of human breast cancer cells expressing the reporter gene firefly luciferase in the cell culture system, and PD-1 immune checkpoint inhibitor is nivolumab or pembrolizumab.

FIG. 15C shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human breast cancer cell MDA-MB-231 (data therein is shown as mean±standard deviation, for all, n=3). Please refer to FIG. 28 and related description according to the present application for the information of each component included in the immune checkpoint PD-1 fusion-based chimeric antigen receptor C #1 version, C #2 version, C #3 version, C #4 version and C #5 version; wherein, the human immunogenic primary T cell in the control group is the one that has not been modified by the chimeric antigen receptor artificial molecular machine, and the target cell survival index represents relative cell number of human breast cancer cells expressing the reporter gene firefly luciferase in the cell culture system.

FIG. 16A shows the establishment and analysis test flow of the cytotoxicity experimental model of the vitro co-culture of T cell and PD-L1 positive human liver cancer cell involved in the present application.

FIG. 16B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human liver cancer cell HA22T (data therein is shown as mean±standard deviation, for all, n=3). Please refer to FIG. 28 and related description according to the present application for the information of each component included in the immune checkpoint PD-1 fusion-based chimeric antigen receptor C #2 version, C #3 version and C #5 version; wherein, the human immunogenic primary T cell in the control group is the one that has not been modified by the chimeric antigen receptor artificial molecular machine, and the target cell survival index represents relative cell number of human liver cancer cells expressing the reporter gene firefly luciferase in the cell culture system.

FIG. 17A shows the establishment and analysis test flow of the cytotoxicity experimental model of the vitro co-culture of T cell and PD-L1 positive human brain cancer cell involved in the present application.

FIG. 17B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human brain cancer cell U87-MG (data therein is shown as mean±standard deviation, for all, n=3). Please refer to FIG. 28 and related description according to the present application for the information of each component included in the immune checkpoint PD-1 fusion-based chimeric antigen receptor C #2 version, C #3 version and C #5 version; wherein, the human immunogenic primary T cell in the control group is the one that has not been modified by the chimeric antigen receptor artificial molecular machine, and the target cell survival index represents relative cell number of human brain cancer cells expressing the reporter gene firefly luciferase in the cell culture system.

FIG. 18A shows the establishment and analysis test flow of the cytotoxicity experimental model of the vitro co-culture of T cell and PD-L1 positive human skin cancer cell involved in the present application.

FIG. 18B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human skin cancer cell A2058 (data therein is shown as mean±standard deviation, for all, n=3). Please refer to FIG. 28 and related description according to the present application for the information of each component included in the immune checkpoint PD-1 fusion-based chimeric antigen receptor C #2 version, C #3 version and C #5 version; wherein, the human immunogenic primary T cell in the control group is the one that has not been modified by the chimeric antigen receptor artificial molecular machine, and the target cell survival index represents relative cell number of human skin cancer cells expressing the reporter gene firefly luciferase in the cell culture system.

FIG. 19A shows the establishment and analysis test flow of the cytotoxicity experimental model of the vitro co-culture of T cell and PD-L1 positive human ovarian cancer cell involved in the present application.

FIG. 19B shows quantitative analysis result of the cytotoxic effect of the in vitro co-culture of human immunogenic primary T cell modified by different immune checkpoint PD-L1 fusion-based chimeric antigen receptor artificial molecular machines and PD-L1 positive human ovarian cancer cell ES-2 (data therein is shown as mean±standard deviation, for all, n=3). Please refer to FIG. 28 and related description according to the present application for the information of each component included in the immune checkpoint PD-1 fusion-based chimeric antigen receptor C #2 version, C #3 version and C #5 version; wherein, the human immunogenic primary T cell in the control group is the one that has not been modified by the chimeric antigen receptor artificial molecular machine, and the target cell survival index represents relative cell number of human ovarian cancer cells expressing the reporter gene firefly luciferase in the cell culture system.

FIG. 20A shows the establishment and analysis test flow of the cytotoxicity experimental model of the vitro co-culture of T cell and PD-L1 positive human prostate cancer cell involved in the present application.

FIG. 20B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human prostate cancer cell PC-3 (data therein is shown as mean±standard deviation, for all, n=3). Please refer to FIG. 28 and related description according to the present application for the information of each component included in the immune checkpoint PD-1 fusion-based chimeric antigen receptor C #2 version, C #3 version and C #5 version; wherein, the human immunogenic primary T cell in the control group is the one that has not been modified by the chimeric antigen receptor artificial molecular machine, and the target cell survival index represents relative cell number of human prostate cancer cells expressing the reporter gene firefly luciferase in the cell culture system.

FIG. 21A shows the establishment and analysis test flow of the cytotoxicity experimental model of the vitro co-culture of T cell and PD-L1 positive human pancreatic cancer cell involved in the present application.

FIG. 21B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human pancreatic cancer cell AsPC1 (data therein is shown as mean±standard deviation, for all, n=3). Please refer to FIG. 28 and related description according to the present application for the information of each component included in the immune checkpoint PD-1 fusion-based chimeric antigen receptor C #2 version, C #3 version and C #5 version; wherein, the human immunogenic primary T cell in the control group is the one that has not been modified by the chimeric antigen receptor artificial molecular machine, and the target cell survival index represents relative cell number of human pancreatic cancer cells expressing the reporter gene firefly luciferase in the cell culture system.

FIG. 22A shows the establishment and analysis test flow of the cytotoxicity experimental model of the vitro co-culture of T cell and PD-L1 positive human colon cancer cell involved in the present application.

FIG. 22B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human colon cancer cell COLO205 (data therein is shown as mean±standard deviation, for all, n=3). Please refer to FIG. 28 and related description according to the present application for the information of each component included in the immune checkpoint PD-1 fusion-based chimeric antigen receptor C #2 version, C #3 version and C #5 version; wherein, the human immunogenic primary T cell in the control group is the one that has not been modified by the chimeric antigen receptor artificial molecular machine, and the target cell survival index represents relative cell number of human colon cancer cells expressing the reporter gene firefly luciferase in the cell culture system.

FIG. 23A shows the establishment and analysis test flow of the cytotoxicity experimental model of the vitro co-culture of T cell and PD-L1 positive human renal cancer cell involved in the present application.

FIG. 23B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human renal cancer cell 786-O (data therein is shown as mean±standard deviation, for all, n=3). Please refer to FIG. 28 and related description according to the present application for the information of each component included in the immune checkpoint PD-1 fusion-based chimeric antigen receptor C #2 version, C #3 version and C #5 version; wherein, the human immunogenic primary T cell in the control group is the one that has not been modified by the chimeric antigen receptor artificial molecular machine, and the target cell survival index represents relative cell number of human renal cancer cells expressing the reporter gene firefly luciferase in the cell culture system.

FIG. 24A shows the establishment and analysis test flow of the cytotoxicity experimental model of the vitro co-culture of T cell and PD-L1 positive human lung cancer cell involved in the present application.

FIG. 24B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human lung cancer cell H441 (data therein is shown as mean±standard deviation, for all, n=3). Please refer to FIG. 28 and related description according to the present application for the information of each component included in the immune checkpoint PD-1 fusion-based chimeric antigen receptor C #2 version, C #3 version and C #5 version; wherein, the human immunogenic primary T cell in the control group is the one that has not been modified by the chimeric antigen receptor artificial molecular machine, and the target cell survival index represents relative cell number of human lung cancer cells expressing the reporter gene firefly luciferase in the cell culture system.

FIG. 25A shows the establishment and analysis test flow of the cytotoxicity experimental model of the vitro co-culture of T cell and PD-L1 positive human lymphoma cancer cell involved in the present application.

FIG. 25B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human lymphoma cancer cell U937 (data therein is shown as mean±standard deviation, for all, n=3). Please refer to FIG. 28 and related description according to the present application for the information of each component included in the immune checkpoint PD-1 fusion-based chimeric antigen receptor C #2 version, C #3 version and C #5 version; wherein, the human immunogenic primary T cell in the control group is the one that has not been modified by the chimeric antigen receptor artificial molecular machine, and the target cell survival index represents relative cell number of human lymphoma cancer cells expressing the reporter gene firefly luciferase in the cell culture system.

FIG. 26A shows the in vitro isolation, infection and expansion process of the lymphatic T cell of the donor mouse used in the present application.

FIG. 26B shows the establishment, monitor and analysis flow and the treatment plan of the mouse syngeneic solid tumor model used in the present application.

FIG. 27A shows quantitative analysis of treatment effects (data therein is shown as mean±standard deviation, for all, n=6) of different immune checkpoint PD-1 fusion-based chimeric antigen receptor artificial molecular machine-modified T cell therapies in a mouse animal model with PD-L1 positive melanoma solid tumor and a well-established immune system. Please refer to FIG. 28 and related description according to the present application for the information of each component included in the immune checkpoint PD-1 fusion-based chimeric antigen receptor C #2 version and C #3 version; wherein, the T cell therapy in the control group refers to use of murine immunogenic primary T cell that has not been modified by chimeric antigen receptor artificial molecular machine, and the tumor volume represents the quantitative volume of solid tumor in the mouse subcutaneous solid tumor model, and the mouse tumor model is a subcutaneous B16 melanoma solid tumor model. Please refer to FIG. 26 for the specific treatment plan flow information.

FIG. 27B shows quantitative analysis of treatment effects (data therein is shown as survival time, for all, n=6) of different immune checkpoint PD-1 fusion-based chimeric antigen receptor artificial molecular machine-modified T cell therapies in a mouse animal model with PD-L1 positive melanoma solid tumor and a well-established immune system. Please refer to FIG. 28 and related description according to the present application for the information of each component included in the immune checkpoint PD-1 fusion-based chimeric antigen receptor C #2 version and C #3 version; wherein, the T cell therapy in the control group refers to use of murine immunogenic primary T cell that have not been modified by chimeric antigen receptor artificial molecular machine, the ordinate of the survival curve refers to the survival rate, the abscissa refers to the survival time, and the mouse tumor model is a subcutaneous B16 melanoma solid tumor model. Please refer to FIG. 26 for the specific treatment plan flow information.

FIG. 27C shows quantitative analysis of treatment effects (data therein is shown as mean±standard deviation, for all, n=6) of different immune checkpoint PD-1 fusion-based chimeric antigen receptor artificial molecular machine-modified T cell therapies in a mouse animal model with PD-L1 positive colon cancer solid tumor and a well-established immune system. Please refer to FIG. 28 and related description according to the present application for the information of each component included in the immune checkpoint PD-1 fusion-based chimeric antigen receptor C #2 version and C #3 version; wherein, the tumor volume represents the quantitative volume of the solid tumor in the mouse subcutaneous solid tumor model, and the mouse tumor model is a subcutaneous MC38 colon cancer solid tumor model. Please refer to FIG. 26 for the specific treatment plan flow information.

FIG. 28 shows a table containing different versions of chimeric protein constructs and showing examples of chimeric proteins including immune checkpoint PD-1 fusion-based chimeric antigen receptor according to the present disclosure.

FIGS. 29A-29B show the vector map of the lentiviral vector, which contains two representative versions: FIG. 29A shows immune checkpoint PD-1 fusion-based chimeric antigen receptor C #3 version and FIG. 29B shows immune check PD-1 fusion-based chimeric antigen receptor C #5 version. Please refer to FIG. 28 and related description according to the present application for the information of each component included in the immune checkpoint PD-1 fusion-based chimeric antigen receptor C #3 version and C #5 version.

FIG. 30 shows the expression of different full versions of the immune checkpoint PD-1 chimeric antigen receptor in monocyte THP1. Compared with the control group, each expression of the chimeric antigen receptors C #2, C #4, C #3 and C #5 fused with different immune checkpoints PD-1 in monocyte THP1 is more than 90%. Chimeric antigen receptors C #2, C #4, C #3 and C #5 fused with different immune checkpoints PD-1 respectively expressed by the monocyte show the efficacy of killing cancer cells in FIGS. 34A-34B to FIG. 37. Please refer to FIG. 28 and related description according to the present application for the information of each component included in the immune checkpoint PD-1 fusion-based chimeric antigen receptor C #2 version, C #4 version, C #3 version and C #5 version.

FIG. 31 shows the expression level of PD-L1 in human lymphoma cancer cell NALM6 modified strain.

FIG. 32 shows the expression levels of PD-L1 in human breast cancer cell MBA-MB-231 and human breast cancer cell MDA-MB-231 pretreated with interferon-γ.

FIG. 33 shows the expression level of PD-L1 in human colorectal cancer cell DLD1 modified strain.

FIG. 34A shows the establishment and analysis test flow of the cytotoxicity experimental model of the vitro co-culture of the human monocyte and PD-L1-positive human lymphoma cancer cell modified strain involved in the present application.

FIG. 34B shows quantitative analysis result of the cytotoxic effect of the in vitro co-culture of human monocyte THP1 modified by different immune checkpoint PD-1 fusion-based chimeric antigen receptor artificial molecular machines and PD-L1 positive human lymphoma cancer cell NALM6 modified strain (data therein is shown mean, for all, n=1); wherein, the human monocyte in the control group is the one that has not been modified by chimeric antigen receptor artificial molecular machine, and the target cell survival index represents relative cell number of human lymphoma cancer cells expressing the reporter gene firefly luciferase in the cell culture system.

FIG. 35A shows the establishment and analysis test flow of the cytotoxicity experimental model of the vitro co-culture of the human macrophage and PD-L1-positive human breast cancer cell involved in the present application.

FIG. 35B shows quantitative analysis result of the cytotoxic effect of the in vitro co-culture of human macrophage modified by different immune checkpoint PD-1 fusion-based chimeric antigen receptor artificial molecular machines and PD-L1 breast cancer cell MDA-MB-231 under the mediation of Erbitux (cetuximab) (data therein is shown mean±standard deviation, for all, n=3); wherein, the human macrophage in the control group is the one that has not been modified by chimeric antigen receptor artificial molecular machine, and the target cell survival index represents relative cell number of human breast cancer cells expressing the reporter gene firefly luciferase in the cell culture system.

FIG. 36A shows the establishment and analysis test flow of the cytotoxicity experimental model of the vitro co-culture of the human macrophage and PD-L1-positive human colorectal cancer cell involved in the present application.

FIG. 36B shows quantitative analysis result of the cytotoxic effect of the in vitro co-culture of human macrophage modified by different immune checkpoint PD-1 fusion-based chimeric antigen receptor artificial molecular machines and PD-L1 positive human colorectal cancer cell DLD1 under the mediation of Erbitux (cetuximab) (data therein is shown mean±standard deviation, for all, n=3); wherein, the human macrophage in the control group is the one that has not been modified by chimeric antigen receptor artificial molecular machine, and the target cell survival index represents relative cell number of human colorectal cancer cells expressing the reporter gene firefly luciferase in the cell culture system.

FIG. 37A shows the establishment and analysis test flow of the cytotoxicity experimental model of the vitro co-culture of the human macrophage and PD-L1-positive human colorectal cancer cell involved in the present application.

FIG. 37B shows quantitative analysis result of the cytotoxic effect of the in vitro co-culture of human macrophage modified by different immune checkpoint PD-1 fusion-based chimeric antigen receptor artificial molecular machines and PD-L1 positive human colorectal cancer cell DLD1 under the mediation of Erbitux (cetuximab) (data therein is shown mean±standard deviation, for all, n=3); wherein, the human macrophage in the control group is the one that has not been modified by chimeric antigen receptor artificial molecular machine, and the target cell survival index represents relative cell number of human colorectal cancer cells expressing the reporter gene firefly luciferase in the cell culture system.

### DETAILED DESCRIPTION OF THE EMBODIMENTS

The present application will be described in detail below with reference to the examples, but the present application is not limited to these examples. The present invention should in no way be construed as limited to the following examples, but should be construed to cover any and all modifications apparent in light of the teachings provided herein.

Without further description, it is believed that the skilled in the art can utilize the foregoing description and the following illustrative examples to make and use the compounds according to the present invention and to practice the claimed method. Accordingly, the following working examples specifically point out preferred embodiments of the invention and are not to be construed as limiting the remainder of the present disclosure in any way.

Unless otherwise specified, the raw materials in the examples of the present application are all commercially available.

The materials and methods used in these experiments are now described.

In the examples of the present application, “molecular machine” and “chimeric antigen receptor” are both proteins, which are exemplary examples of the present invention.

According to one aspect of the present application, a chimeric antigen receptor is constructed which comprises:
a) an extracellular target molecule binding domain for specifically binding to a target molecule;
b) an intracellular signaling domain comprising at least one intracellular activation signaling domain and/or at least one intracellular detection signaling domain; and
c) a transmembrane domain, used to connect the extracellular target molecule binding domain and the intracellular signaling domain, and anchor these two domains on a cell membrane;
wherein, an activation of the intracellular activation signaling domain at least relies on the binding of the extracellular target molecule binding domain to the target molecule; and the intracellular activation signaling domain contains a molecule or a fragment having a catalytic domain.

The target molecule recognized by the chimeric antigen receptor can be at least one of such as immunosuppressive signal-related molecules or tumor surface antigen molecular markers. The extracellular target molecule binding domain is at least one of molecules that can identify and bind target molecules such as immunosuppressive signal-related molecules or tumor surface antigen molecular markers, or can be common monoclonal antibody or single-chain variable fragment and antigen-recognition-binding fragment thereof in existing chimeric antigen receptor, anti-immunosuppressive signal-related molecule monoclonal antibody and the antigen-recognition-binding fragment thereof, or anti-tumor surface antigen molecular marker monoclonal antibody and the antigen-recognition-binding fragment thereof. Preferably, the extracellular target molecule binding domain is at least one of molecules that can identify and bind to immunosuppressive signal-related molecules or tumor surface antigen molecular markers.

Intracellular signaling domain comprises at least one intracellular activation signaling domain which is preferably immune cell activation signaling domain, and an activation of the intracellular activation signaling domain at least relies on the binding of the extracellular target molecule binding domain to the target molecule, and the intracellular activation signaling domain contains a molecule having a catalytic domain. The intracellular signaling domain contains a molecule with a catalytic domain or a fragment thereof, which enables the chimeric antigen receptor to break away from the confinement of a specific cell type and expands to cell types with applicability to a molecule with a catalytic domain, that is, expands the range of host cell types that the chimeric antigen receptor described in the present application can confer genetic modification to express the chimeric antigen receptor.

In certain such embodiments, expression of the chimeric antigen receptor as described in the present application confers an immune activation phenotype and/or a phagocytic phenotype to host cells that do not naturally exhibit an immune activation phenotype. In other such embodiments, the host cell expressing chimeric antigen receptor as described in the present application confers an immune activation phenotype and/or a phagocytic phenotype specific for an antigenic marker not naturally targeted by the host cell. In yet other such embodiments, the host cell expressing the chimeric antigen receptor as described in the present application confers an immune activation phenotype and/or a phagocytic expression specific for the antigenic marker targeted by the host cell, and the host cell expressing the chimeric antigen receptor enhances the host cell's immune activation, recognition and killing, and/or recognition and phagocytosis to the cell, microorganism or particle that displays the antigenic marker.

For transmembrane domain, the transmembrane protein can be used, and no other requirements are needed.

Based on the application scenario related to PD-1/PD-L1 immunosuppressive signal, the hypothesis of the chimeric antigen receptor molecular machine is verified. Considering the advantages and disadvantages of immune checkpoint inhibitor and cell therapy described in the background, especially the challenges faced in the treatment of solid tumors, for example solid tumors have a complex immunosuppressive tumor microenvironment, a new generation of cell therapy of solid tumors with chimeric antigen receptor based on the immune checkpoint PD-1 signaling pathway is proposed and developed. The technology combines various methods such as tumor immunology, synthetic biology, molecular engineering and cell engineering to establish and apply an immune checkpoint PD-1 based chimeric antigen receptor artificial molecular machine which encodes and regulates the immune cell function, which possesses advantages of immune checkpoint inhibitor and cell therapy, providing solutions for overcoming the immunosuppression of the tumor microenvironment and improving the treatment of the solid tumor.

When the cancer cells expressing the immune checkpoint inhibitory signal PD-L1 as PD-1 molecule ligand try to inhibit the function of immune T cells or phagocytes by means of brake blocking mechanism in the immune checkpoint PD-1/PD-L1 signaling pathway, the immune T cells or phagocytes modified by re-encoding with this new generation of immune checkpoint PD-1-based chimeric antigen receptor molecular machine will not be inhibited by PD-L1 positive cancer cells, but will specifically recognize the PD-L1 positive cancer cells and be further activated, generating immune activation phenotype and specific immune response against the corresponding cancer cells, thereby extremely efficiently identifying and killing the corresponding cancer cells.

### Definition

Before the present disclosure is set forth in greater detail, definitions of certain terms used in the present disclosure are provided that may assist in understanding the present disclosure.

Phagocytosis: The term “phagocytosis” as used in the present application is defined as a receptor-mediated process in which endogenous or exogenous cells or particles greater than 100 nm in diameter are internalized by phagocytes or host cells of the present disclosure. Phagocytosis typically consists of multiple steps: (1) tethering a target cell or particle, by a phagocytosis receptor either directly or indirectly (through bridging molecules) binding to pro-phagocytic marker or antigenic marker on the target cell or particle; and (2) internalization or phagocytosis of whole target cell or particle or a part thereof. In certain embodiments, internalization can occur through rearrangement of the cytoskeleton of a phagocyte or host cell to form a phagosome (a membrane-binding compartment containing an internalization target). Phagocytosis can also comprise maturation of the phagosome, wherein the phagosome becomes more acidic and fuses with a lysosome (to form a phagolysosome), followed by degradation of the phagocytosed target (eg, “phagocytosis”). Alternatively, phagosome-lysosome fusion may not be observed in phagocytosis. In yet another embodiment, the phagosome can reflux or expel its contents into the extracellular environment prior to complete degradation. In some embodiments, phagocytosis refers to phagocytosis. In some embodiments, phagocytosis comprises that the phagocyte as host cell tethers the target cell or particle, but do not internalize the same. In some embodiments, phagocytosis comprises that the phagocyte as host cell tethers the target cell or particle, and internalizes part of the same.

Extracellular target molecule binding domain: the term “target molecule binding domain” as used in the present application is defined as the molecule (such as peptide, oligopeptide, polypeptide or protein) having the ability to specifically and non-covalently bind, associate, unite, or recognize a target molecule such as PD-1, IgG antibody, IgE antibody, IgA antibody, CD138, CD38, CD33, CD123, CD79b, mesothelin, PSMA, BCMA, ROR1, MUC-16, L1CAM, CD22, CD19, EGFRviii, VEGFR-2 or GD2. The target molecule binding domain comprises any natural, synthetic, semi-synthetic or recombinantly produced binding partner for a target biomolecule or other target. In some embodiments, the target molecule binding domain is an antigen binding domain, such as an antibody, a domain having antigen binding function thereof or antigen binding portion. Exemplary binding domains comprise single chain antibody variable region (e.g., domain antibody, sFv, scFv, Fab), receptor extracellular domain (e.g., PD-1), ligand (e.g., cytokine, chemokine), or synthetic polypeptide selected for their specific binding ability to biomolecule.

Intracellular signaling domain: the term “intracellular signaling domain” as used in the present application is defined as an intracellular effector domain; the intracellular activation signaling domain is fully released and activated based on the conformational change of the chimeric antigen receptor molecular machine, and the activation signaling domain under the activation state can further activate various downstream signaling pathways, so as to make the immune cell modified by the chimeric antigen receptor perform specific functions against the target cell, such as the killing function of immune T cell against cancer cell, or the phagocytosis and killing function of the phagocyte against the cancer cell under the condition that the extracellular target molecule binding domain of the chimeric antigen receptor molecular machine on the surface of an immune cell recognizes and binds to the target molecule so as to provide input of the target molecule recognition and binding signal through this recognition and binding, then the molecular conformation of the intracellular part changes to unwind its activation signaling domain from the autoinhibited molecular conformation state and finally respond to the input of the upstream target molecule recognition and binding signal. In certain embodiments, an activation of the signaling domain causes one or more signaling pathways that lead to the killing of target cell, microorganism or particle by the host cell. In certain embodiments, the signaling domain comprises at least one intracellular activation signaling domain. In certain other embodiments, the signaling domain comprises at least one intracellular detection signaling domain and at least one intracellular activation signaling domain. In certain other embodiments, the signaling domain comprises at least one intracellular detection signaling domain, an intracellular linker domain, and at least one intracellular activation signaling domain.

Intracellular activation signaling domain: the term “intracellular activation signaling domain” as used in the present application is defined as being non-receptor tyrosine kinase or receptor tyrosine kinase molecule or fragment having catalytic function, which are capable of promoting, directly or indirectly, a biological or physiological response in a cell expressing an activation signaling domain when received an appropriate signal. In certain embodiments, the activation signaling domain is part of a protein or protein complex that receives a signal upon binding. For example, in response to the binding of the PD-1-fused chimeric antigen receptor to the target molecule PD-L1, the activation the signaling domain can transmit signals to the interior of the host cell and stimulate effector function, such as T cell effectively killing cancer cell, phagocytosis of cancer cell by phagocyte, phagolysosome maturation, secretion of anti-inflammatory and/or immunosuppressive cytokine, secretion of inflammatory cytokine and/or chemokine. In other embodiments, the activation signaling domain will promote a cellular response indirectly by binding to one or more other proteins that directly promotes the cellular response.

Detection signaling domain: the term “detection signaling domain” as used in the present application is defined as the immunoreceptor tyrosine-based activation motif (ITAM) which is conserved sequence consisting of more than ten amino acids. When the tyrosine kinase activation signal is input, the detection signaling domain of the chimeric antigen receptor molecular machine will respond to the signal input and undergo phosphorylation modification, and then the phosphorylation-modified detection signaling domain will interact with the activation signaling structure based on phosphorylation site modification, thereby unwinding the activation signaling domain from its autoinhibited molecular conformational state, releasing the activation signaling domain, and the activation signaling domain of the molecular machine under the molecular conformation after the activation signaling domain is released is in open activation state. The primary detection signaling sequence can comprise a signal motif known as an immunoreceptor tyrosine-based activation motif (ITAM). ITAM is well-defined signaling motif found in the intracytoplasmic tails of various receptors that serve as binding site for tyrosine kinase. Examples of ITAM used in the present invention can include as non-limiting examples those derived from 2B4, CD244, BTLA, CD3δ, CD3γ, CD3ε, CD3ζ, CD5, CD28, CD31, CD72, CD84, CD229, CD300a, CD300f, CEACAM-1, CEACAM-3, CEACAM-4, CEACAM-19, CEACAM-20, CLEC-1, CLEC-2, CRACC, CTLA-4, DAP10, DAP12, DCAR, DCIR, Dectin-1, DNAM-1, FcεRIα, FcεRIβ, FcγRIB, FcγRI, FcγRIIA, FcγRIIB, FcγRIIC, FcγRIIIA, FCRL1, FCRL2, FCRL3, FCRL4, FCRL5, FCRL6, G6b, KIR, KIR2DL1, KIR2DL2, KIR2DL3, KIR2DL4, KIR2DL5, KIR2DL5B, KIR2DS1, KIR2DS3, KIR2DS4, KIR2DS5, KIR3DL1, KIR3DL2, KIR3DL3, KIR3DS1, KLRG1, LAIR1, LILRB1, LILRB2, LILRB3, LILRB4, LILRB5, MICL, NKG2A, NKp44, NKp65, NKp80, NTB-A, PD-1, PDCD6, PILR-α, Siglec-2, Siglec-3, Siglec-5, Siglec-6, Siglec-7, Siglec-8, Siglec-9, Siglec-10, Siglec-11, Siglec-12, Siglec-14, Siglec-15, Siglec-16, SIRPα, SLAM, TIGIT, TREML1, and TREML2.

Intracellular spacer domain is located between the transmembrane domain and the intracellular signaling domain and connects these two domains together, and can be an extension of the transmembrane domain.

Transmembrane domain: the term “transmembrane domain” as used in the present application is defined as a polypeptide that spans the entire biological membrane once and serves to connect the extracellular target molecule binding domain and the intracellular signaling structure domain and anchor these two domains on the cell membrane.

Intracellular linker domain: the term “intracellular linker domain” as used in the present application is defined as connecting the detection signaling domain and the intracellular activation signaling domain, and can be optionally a flexible linker peptide fragment. The linker domain can provide the desired flexibility to allow the desired expression, activity and/or conformational positioning of the chimeric polypeptide. The intracellular linker domain can be of any suitable length to connect at least two domains of interest, and is preferably designed to be flexible enough to allow proper folding and/or function and/or activity of one or both domains to which it connects. The length of the intracellular linker domain is at least 3, 5, 10, 15, 20, 25, 30, 35, 40, 45, 50, 55, 60, 65, 70, 75, 80, 85, 90, 95, 90, 95 or 100 amino acids. In some embodiments, the length of intracellular linker domain is about 0 to 200 amino acids, about 10 to 190 amino acids, about 20 to 180 amino acids, about 30 to 170 amino acids, about 40 to 160 amino acids, about 50 to 150 amino acids, about 60 to 140 amino acids, about 70 to 130 amino acids, about 80 to 120 amino acids, or about 90 to 110 amino acids. In some embodiments, the intracellular linker domain can comprise endogenous protein sequence. In some embodiments, the intracellular linker domain comprises glycine, alanine and/or serine residues. In some embodiments, the intracellular linker domain can comprise a motif, such as multiple or repeated motifs of GS, GGS, GGGGS, GGSG or SGGG. The linker domain sequence can include any natural amino acid, non-natural amino acid, or a combination thereof.

Sequence Homology: the term “sequence homology” used in the present application is defined as the significant similarity in terms of coding sequence among two or more nucleic acid molecules or among two or more protein sequences, for example having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or at least 100% sequence coding identity.

Host cell: the term “host cell” as used in the present application is defined as a cell (such as lymphocyte) capable of receiving and accommodating recombinant molecules, and is a site for the expansion and expression of recombinant genes.

Phase-contrast imaging is a technology for imaging based on the phase-contrast method.

PD-L1-binding fragment: the term “PD-L1-binding fragment” as used in the present application is defined as a molecule or molecular fragment (such as antibody fragment) that has the ability to specifically bind to PD-L1.

Tumor microenvironment refers to the surrounding microenvironment in which cancer cells exist, including surrounding blood vessels, immune cells, fibroblasts, bone marrow-derived inflammatory cells, various signaling molecules and extracellular matrix. Tumors and the surrounding environment are closely related and interact continuously. Tumors can affect their microenvironment by releasing cell signaling molecules, promoting tumor angiogenesis and inducing immune tolerance, while immune cells in the microenvironment can affect the growth and development of cancer cells. The tumor microenvironment promotes the formation of tumor heterogeneity.

Catalytic function: many chemical reactions in the body are carried out by enzymes which are used as catalyst to accelerate the speed of chemical reactions, that is, which have catalytic functions; wherein, tyrosine kinase is an enzyme that catalyzes the transfer of phosphate group from ATP to tyrosine residue of protein in a cell, and regulates the “on” and “off” of signaling pathway in the cell. Tyrosine kinase as used in the present application comprises ZAP70 and SYK.

Conformation refers to a spatial arrangement generated by the placement of the atoms around only single bond of a molecule that does not change the structure of covalent bond. Different conformations can be transformed into each other. Among various conformational forms, the one with the lowest potential energy and the most stability is the dominant conformation. Breaking and reforming of the covalent bond is not required when one conformation is transformed to another. The molecule conformation affects not only the physical and chemical properties of a compound, but also the structure and properties of some biological macromolecules (such as protein, enzyme, nucleic acid).

Immunosuppressive signal-related molecule: immune checkpoint can be stimulatory or inhibitory signal-related molecule, wherein costimulatory protein can transmit signal to promote immune response against pathogen while inhibitory one does the opposite. For example, inhibitory signal-related molecule can be cytotoxic T lymphocyte-associated antigen 4 (CTLA-4) and programmed cell death receptor 1 (PD-1) and its ligand PD-L1, which are the most studied several immunosuppressive signal-related molecules so far.

Specific antigen peptide-histocompatibility complex molecule on cell surface: in the antigen presentation pathway, these epitope peptides must be cleaved by the proteasome, then bind to the transfer protein associated with antigen processing (TAP), and finally bind to the major histocompatibility complex (MEC) molecule in endoplasmic reticulum and further successfully transported to the surface of the antigen presentation molecule, thereby obtaining the specific antigen peptide-histocompatibility complex molecule which then presents the specific antigen peptide on the surface thereof and is recognized by associated immune cells.

Truncation: the term “truncation” as used in the present application is defined as a shortened fragment due to deletion of a sequence.

Protein mutant: the term “protein mutant” as used in the present application is defined as altering the amino acid sequence of the original protein in order to obtain a functional or non-functional mutant protein.

Immune checkpoint: immune checkpoint refers to the intrinsic regulatory mechanism-related molecule of the immune system that maintains self-tolerance and helps avoid collateral damage during physiological immune response, such as immune checkpoint PD-1 and CTLA-4. Today, it is apparent that tumors can establish microenvironment to evade immune surveillance and attack, particularly by modulating certain immune checkpoint pathway.

Immunosuppression refers to the inhibition against the immune response, that is, the body may not produce an immune response to its own tissue components to maintain self-tolerance, and also refers to the specific non-response state of the immune system against specific antigen.

Tumor immune escape refers to the phenomenon that cancer cells can survive and proliferate in the body by evading the recognition and attack of the body's immune system through various mechanisms. The immune system of the body has the function of immune surveillance. When malignant cells appear in the body, the immune system can recognize and specifically remove these “non-self” cells through the immune mechanism to resist the occurrence and development of the tumor. However, under some situations, malignant cells can escape the immune surveillance of the body through various mechanisms, rapidly proliferate in the body and form tumor.

Macrophage: macrophage is important immune cell in the body, with important role in anti-infection, anti-tumor and immune regulation. The first role refers to anti-infection: non-specific phagocytosis and killing of a variety of pathogenic microorganisms, and thus macrophage is an important cell in the body's non-specific immune defense. The second role refers to presenting antigen and initiating immune response: in a specific immune response, most antigens need to be phagocytosed and processed by macrophages, and form specific antigen peptide-histocompatibility complex molecules with the histocompatibility complex molecules on their surface, express on the cell membrane surface and then present to T cells.

Monocyte: monocyte is the largest blood cell in the blood and the largest white blood cell, and is an important part of the body's defense system. Monocyte is derived from hematopoietic stem cell in the bone marrow, develops in the bone marrow, and is still immature cell when they enter the blood from the bone marrow. It is currently believed to be that the monocyte is the precursor of macrophage and dendritic cell, with obvious deformation movement, which can phagocytose and clear injured and senescent cells and their debris.

Nivolumab (the product name thereof is Opdivo, and the Chinese trade name is also Opdivo) can inhibit PD-1, prevent PD-L1 from binding to PD-1, thereby improving the immunogenicity of cancer cells and enabling T cells to exert the role of immunity surveillance to clear cancer cells. As a first-line drug for clinical use, it is the first PD-1 inhibitor to be included in the WHO Model List of Essential Medicines.

Pembrolizumab: (the trade name thereof is Keytruda and the Chinese trade name is Keytruda or Jishuda) is a human monoclonal antibody that can bind to and block the immune checkpoint PD-1 located on the lymphocyte. The drug was approved by the FDA in the United States in 2014 for any unresectable or metastatic solid tumor.

Chimeric: the term “chimeric” as used in the present application is defined as any nucleic acid molecule or protein that is non-endogenous and comprises sequences that are bound or connected together (not typically bound or connected in nature). For example, a chimeric nucleic acid molecule can comprise regulatory and coding sequences from different sources, or from the same source but arranged in a manner different from that found in nature.

Adoptive Cell Therapy: The term “adoptive cell therapy” as used in the present application is defined as an individualized treatment method that utilizes a patient's own immune cells to attack their specific cancer cells. Chimeric antigen receptor T-cell (CAR-T) cell therapy is a type of adoptive cell therapy that uses genetically modified T cells to fight cancer. The patient's T cells are isolated and collected by apheresis of lymphocytes, modified to produce a special antibody structure of chimeric antigen receptor on their surface, and then infused back to the patient's body. The modified CAR-T cell can target specific antigen on the surface of cancer cell, thereby killing the cancer cell.

Irradiation: the term “irradiation” as used in the present application is defined as a chemical technology that utilizes radiation of radioactive elements to alter molecular structure.

“Nucleic acid molecule” and “polynucleotide”: the terms “nucleic acid molecule” and “polynucleotide” as used in the present application are defined as form of RNA or DNA, which comprises cDNA, genomic DNA, and synthetic DNA. Nucleic acid molecule may be double-stranded or single-stranded, and if single-stranded, it can be coding or non-coding (antisense) strand. A coding molecule may have the same coding sequence as known in the art, or may have a different coding sequence which is capable of encoding the same polypeptide due to redundancy or degeneracy of the genetic code.

“Positive”: the term “positive” as used in the present application is defined as a certain level of expression of a specific molecular marker by a specific cell. For example, PD-L1 positive cancer cell refers to cancer cell that expresses a certain level of PD-L1 protein molecule.

“High expression”: the term “high expression” as used in the present application is defined as a high level of expression of a specific molecular marker by a specific cell. For example, the cancer cell with high PD-L1 expression refer to the one that has high expression levels of PD-L1 protein molecule. Highly expressed cancer cell marker is often associated with disease state, such as in hematological malignancy and in cell that form solid tumor within a particular tissue or organ of a subject. Hematological malignancy or solid tumor characterized by high expression of tumor marker can be determined by standard assay well known in the art.

Cancer: the term “cancer” as used in the present application is defined as a disease characterized by the rapid and uncontrolled growth of abnormal cells. Abnormal cells can form solid tumor or constitute hematological malignancy. Cancer cell can spread locally or through the bloodstream and lymphatic system to other parts of the body. Examples of various cancers include, but are not limited to, breast cancer, prostate cancer, ovarian cancer, cervical cancer, skin cancer, pancreatic cancer, colorectal cancer, kidney cancer, liver cancer, brain cancer, lymphoma cancer, leukemia and lung cancer.

Treatment: the term “treatment” as used in the present application is defined as a method of obtaining a beneficial or desired clinical effect. For the purpose of the present invention, beneficial or desired clinical effects include, but are not limited to, one or more of the following: reducing tumor or cancer cell proliferation (or destroying tumors or cancer cells), inhibiting cancer cell metastasis, enabling tumors expressing PD-L1 to shrink or reduce in size, achieving regression of PD-L1-related disease (e.g., cancer), reducing symptoms due to PD-L1-related diseases (e.g., cancer), improving the life quality of those patients with PD-L1-related disease (e.g., cancer), reducing the dose of other drugs required to treat PD-L1-related disease (e.g., cancer), delaying progression of PD-L1-related disease (e.g., cancer), curing PD-L1-related disease (e.g., cancer), and/or extending survival time of patients with PD-L1-related disease (e.g., cancer).

Vector: the term “vector” as used in the present application is defined as a nucleic acid molecule capable of transporting another nucleic acid. The vector can be, for example, plasmid, cosmid, virus or phage. The term should also be interpreted to include non-plasmid and non-viral compound that facilitates transfer of nucleic acid into a cell. “Expression vector” refers to a vector capable of directing the one or more genes carried by the vector to express a protein when present in a suitable environment. In certain embodiments, the vector is a viral vector. Examples of viral vectors include, but are not limited to, adenoviral vector, adeno-associated viral vector, retroviral vector, γ retroviral vector, and lentiviral vector. “Retrovirus” is a virus with an RNA genome. “γ retrovirus” refers to a genus of the retroviridae. Examples of γ retrovirus include mouse stem cell virus, mouse leukemia virus, feline leukemia virus, feline sarcoma virus, and avian reticuloendotheliosis virus. “Lentivirus” refers to a genus of retroviruses capable of infecting dividing and non-dividing cells. Examples of lentivirus include, but are not limited to, HIV (human immunodeficiency virus, including HIV type 1 and type 2), equine infectious anemia virus, feline immunodeficiency virus (FIV), bovine immunodeficiency virus (BIV), and simian immunodeficiency virus (SIV). In other embodiments, the vector is a non-viral vector. Examples of non-viral vectors include lipid-based DNA vector, modified mRNA (modRNA), self-amplifying mRNA, closed linear duplex (CELiD) DNA, and transposon-mediated gene transfer (PiggyBac, Sleeping Beauty). When a non-viral delivery system is used, the delivery vehicle can be a liposome. Nucleic acid can be introduced into host cell in vitro, ex vivo or in vivo using lipid formulations. Nucleic acid can be encapsulated inside liposome, interspersed within the lipid bilayer of the liposome, attached to liposome by connecting molecule that binds liposome to nucleic acid, contained within micelles or complexed with micelle or otherwise bound to lipid.

Other definitions are used throughout the present disclosure.

### Example 1 Construction and Expression of Chimeric Antigen Receptor

Construction of chimeric antigen receptor molecular machine fused with immune checkpoint PD-1 and carrier.

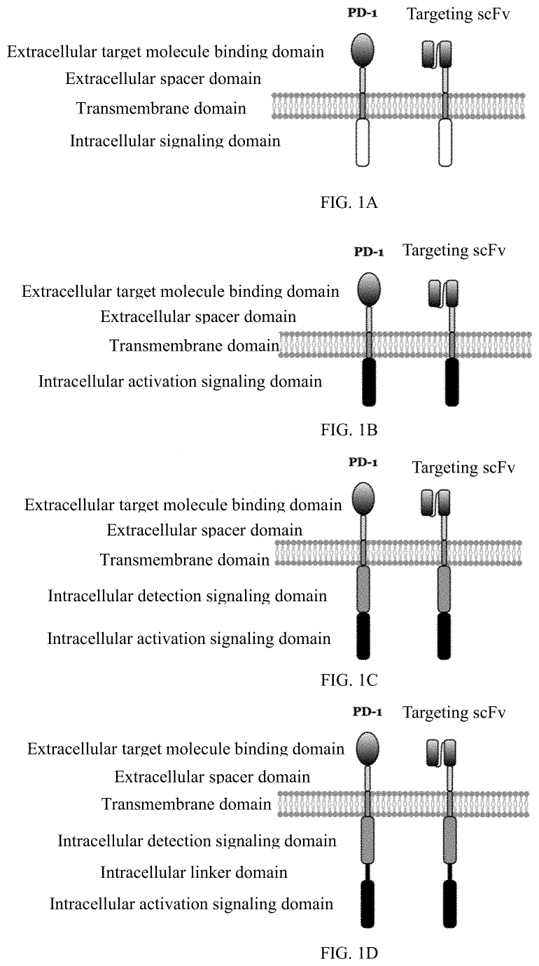

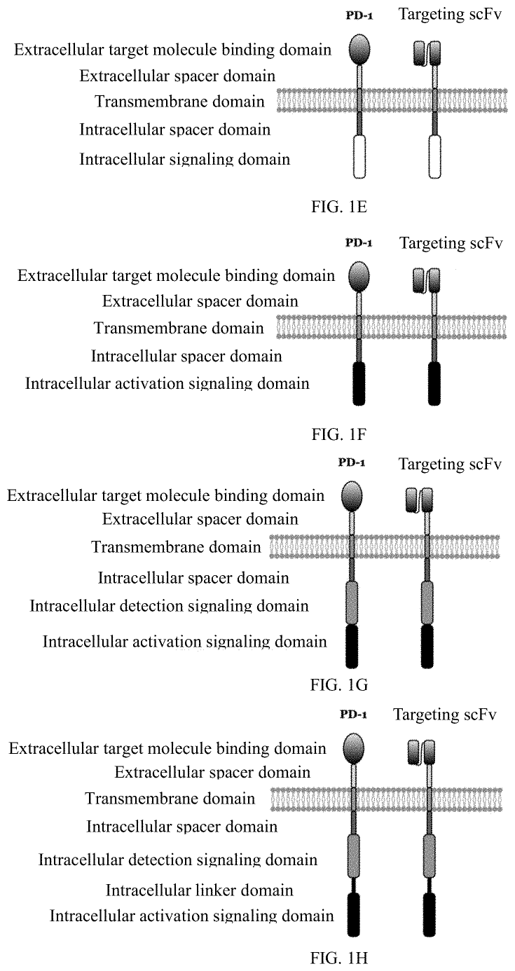

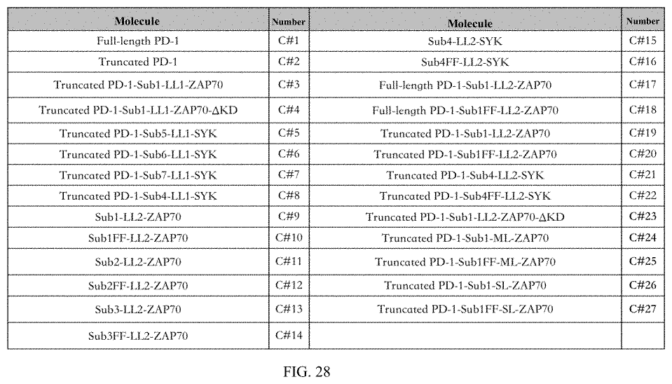

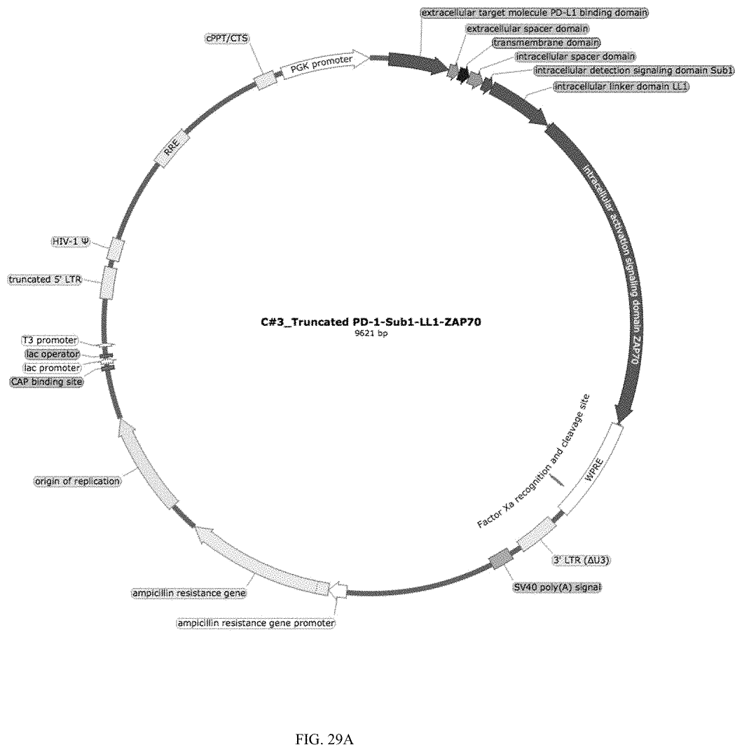

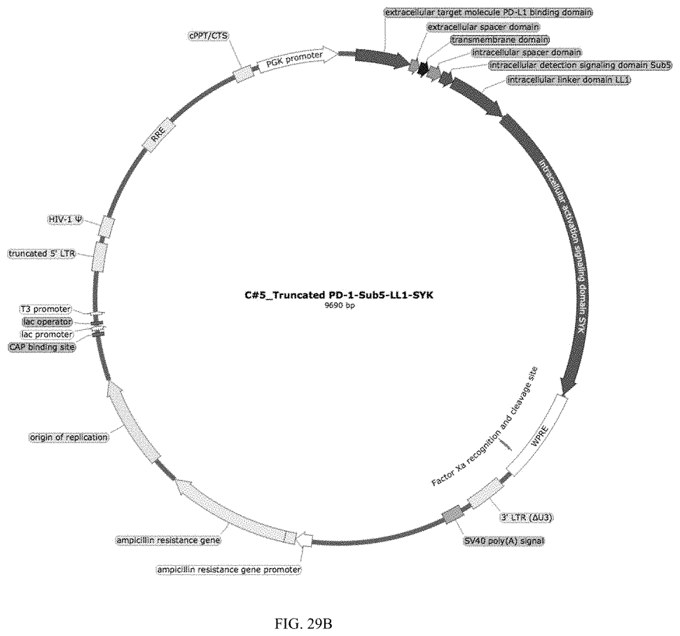

(1) The intracellular signaling domain of the intracellular part of the chimeric antigen receptor (including the intracellular activation signaling domain as the activation element, the intracellular detection signaling domain as the detection element and the intracellular linker domain as connecting element) and the extracellular target molecule binding domain, the transmembrane domain, the extracellular spacer domain and intracellular spacer domain (see FIGS. 1A-AH) as extracellular recognition elements are subjected to ligation fusion through genetic engineering, using Gibson Assembly for seamless cloning ligation, and finally cloned into a specific gene expression vector (such as pSIN lentiviral vector or pMSCV retroviral vector or pCAG or pCDNA3) for subsequent in vitro and in vivo studies. As shown in FIG. 1H, the extracellular target molecule binding domain can be selected as the recognition binding part of PD-L1 as ligand of the receptor PD-1, the extracellular spacer domain can be selected as the extracellular extension fragment of the transmembrane region of PD-1 (that is, between the extracellular target molecule PD-L1 binding domain and the transmembrane region of PD-1), the transmembrane domain can be selected as the transmembrane region of PD-1, the intracellular spacer domain can selected as an intracellular extension fragment of the transmembrane region of PD-1 (i.e. the intracellular portion of the Full-length PD-1 or Truncated PD-1 in FIG. 28, in which the full length of amino acid sequence of Truncated PD-1 in C #2 is SEQ ID NO:001+SEQ ID NO:016+SEQ ID NO:012+SEQ ID NO:054), the intracellular detection signaling domain can be selected as immunoreceptor tyrosine-based activation motif fragment (i.e. Sub1˜Sub7 in FIG. 28: CD3ζ ITAM1˜3, CD3ε ITAM, FcRIIA ITAM, FcRγ ITAM, DAP12 ITAM) of CD3ζ, CD3ε, FcRIIA, FcRγ, DAP12 and other molecules, the intracellular activation signaling domain can be selected as tyrosine kinase of SYK/ZAP70 family member and the like, and the intracellular linker domain used to connect the intracellular detection signaling domain and the intracellular activation signaling domain can be selected as flexible linker peptide fragment (i.e. the linker peptides with different lengths in FIG. 28: SL, ML, LL1, LL2), see FIGS. 1A-1H and FIG. 28. Various versions of the chimeric antigen receptor molecular machines listed in FIG. 28 are constructed respectively, including immune checkpoint PD-1 fusion-based chimeric antigen receptor: C #1 Full-length PD-1, C #2 Truncated PD-1, C #3 Truncated PD-1-Sub1-LL1-ZAP70, C #4 Truncated PD-1-Sub1-LL1-ZAP70-ΔKD, C #5 Truncated PD-1-Sub5-LL1-SYK, C #6 Truncated PD-1-Sub6-LL1-SYK, C #7 Truncated PD-1-Sub7-LL1-SYK, C #8 Truncated PD-1-Sub4-LL1-SYK, C #9 Sub1-LL2-ZAP70, C #10 Sub1FF-LL2-ZAP70, C #11 Sub2-LL2-ZAP70, C #12 Sub2FF-LL2-ZAP70, C #13 Sub3-LL2-ZAP70, C #14 Sub3FF-LL2-ZAP70, C #15 Sub4-LL2-SYK, C #16 Sub4FF-LL2-SYK, C #17 Full-length PD-1-Sub1-LL2-ZAP70, C #18 Full-length PD-1-Sub1FF-LL2-ZAP70, C #19 Truncated PD-1-Sub1-LL2-ZAP70, C #20 Truncated PD-1-Sub1FF-LL2-ZAP70, C #21 Truncated PD-1-Sub4-LL2-SYK, C #22 Truncated PD-1-Sub4FF-LL2-SYK, C #23 Truncated PD-1-Sub1-LL2-ZAP70-ΔKD, C #24 Truncated PD-1-Sub1-ML-ZAP70, C #25 Truncated PD-1-Sub1FF-ML-ZAP70, C #26 Truncated PD-1-Sub1-SL-ZAP70 and C #27 Truncated PD-1-Sub1FF-SL-ZAP70.

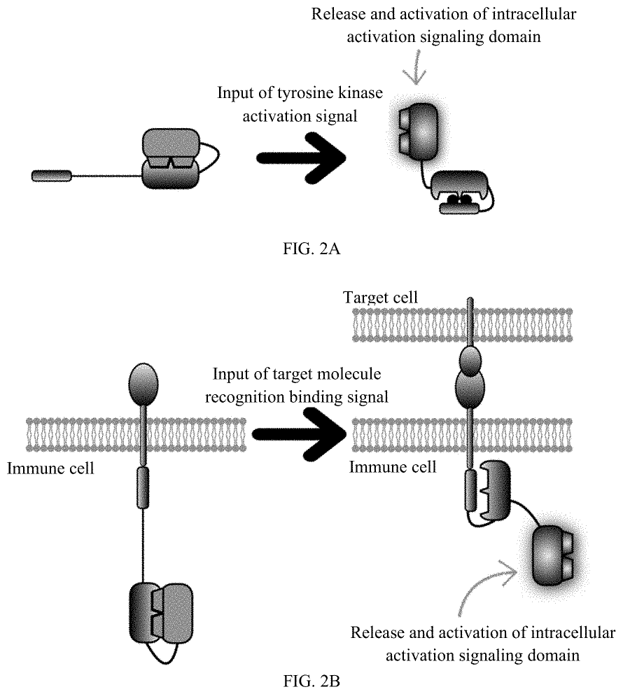

(2) Through the method of DNA lipofection or DNA electroporation transfection, immune checkpoint PD-1 fusion-based chimeric antigen receptor artificial molecular machines with different expressions can be realized in specific cell. Then, fluorescence microscopy imaging is used to detect the expression distribution of different designs of immune checkpoint PD-1 fusion-based chimeric antigen receptor artificial molecular machines in human HeLa cells, mouse embryonic fibroblasts MEFs and human Jurkat E6-1 cells, and their performance in response to a variety of different external stimulus input signals, see FIGS. 2A-2B and FIGS. 6A-6C to FIG. 11. Human HeLa cells and mouse embryonic fibroblasts MEFs are cultured in DMEM culture medium containing 10% fetal bovine serum, and human Jurkat E6-1 cells are cultured in RPMI culture medium containing 10% fetal bovine serum.

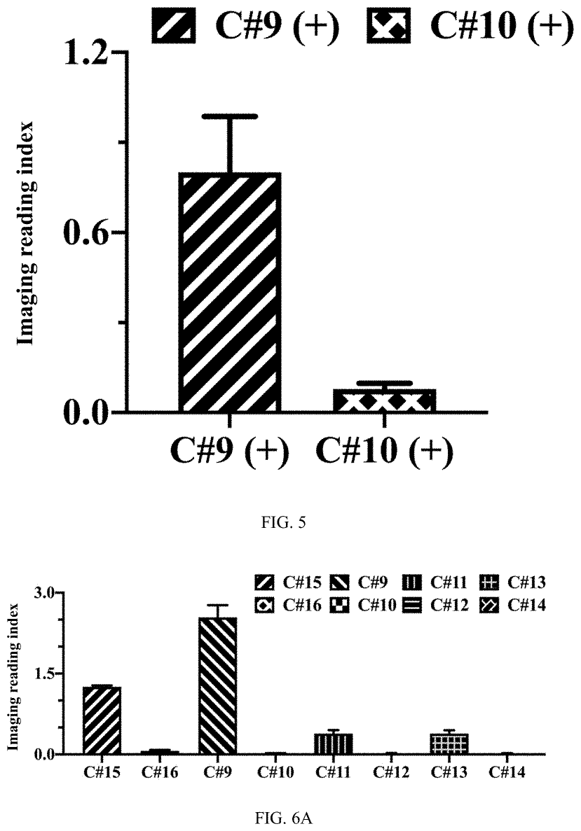

On the other hand, through DNA lipofection, different chimeric antigen receptor proteins are expressed in human 293T cells, isolated and purified, and then the purified proteins are used for extracellular functional test and verification, especially comparison of the effects of different intracellular detection signaling domains and intracellular activation signaling domains on specific protein tyrosine phosphorylation signal input, see FIG. 2A and FIG. 5. Human 293T cells are cultured in DMEM culture medium containing 10% fetal bovine serum.

### Example 2 Detection and Characterization of Chimeric Antigen Receptor

Combining the information provided in FIGS. 1A-1H and FIGS. 2A-2B, a variety of detection and characterization solutions for artificial molecular machines are provided, including but not limited to, detection and characterization of the functional performance of chimeric antigen receptor in eukaryotic cell by different means, and detection and characterization of extracellular functional performance of chimeric antigen receptor in the form of purified proteins.

FIGS. 2A-2B show a schematic diagram of the signal activation of the chimeric antigen receptor artificial molecular machine comprising an extracellular target molecule binding domain, wherein (a) is a schematic diagram of the signal activation of the artificial molecular machine under the condition that the tyrosine kinase activation signal is input, (b) is a schematic diagram of signal activation of the chimeric antigen receptor artificial molecular machine comprising an extracellular target molecule binding domain (such as the extracellular portion of PD-1) under the condition that target molecule recognition binding signal (such as PD-L1) is input.

The working model of the molecular machine in FIG. 2A is a simplified model, that is, it consists of three parts: the detection signaling domain, the linker domain and the activation signaling domain. The detection signaling domain can be selected as immunoreceptor tyrosine-based activation motif fragment (i.e. Sub1˜Sub7 in FIG. 28: CD3ζ ITAM1˜3, CD3ε ITAM, FcRIIA ITAM, FcRγ ITAM, DAP12 ITAM) of CD3ζ, CD3ε, FcRIIA, FcRγ, DAP12 and other molecules, the activation signaling domain can be selected as tyrosine kinase of SYK/ZAP70 family member and the like, and the linker domain used to connect the detection signaling domain and the activation signaling domain can be selected as flexible linker peptide fragment.

Based on the characteristics of the molecular conformation of SYK/ZAP70 family member, in its inactive state, SYK or ZAP70 can be in an auto-inhibited molecular conformation state (Yan Q et al., Molecular and cellular biology. 2013 Jun. 1; 33(11): 2188-201.), and the activation signaling domain of the molecular machine in this conformation is in a closed inactive state. When the tyrosine kinase activation signal is input, especially the phosphorylation signal of the immunoreceptor tyrosine-based activation motif is input, the detection signaling domain of the molecular machine will respond to the signal input and undergo phosphorylation modification, and then the phosphorylation-modified detection signaling domain will interact with SYK or ZAP70 based on phosphorylation site modification, especially under the condition that the flexible linker peptide fragment of the linker domain provides sufficient flexibility in the conformational change of the molecular machine, thereby unwinding the activation signaling domain from its autoinhibited molecular conformational state, releasing the activation signaling domain, and the activation signaling domain of the molecular machine under the molecular conformation after the activation signaling domain is released is in open activation state. That is, the schematic diagram of the signal activation of the artificial molecular machine under the condition that a tyrosine kinase activation signal is input as shown in FIG. 2A, and the activation signaling domain in the active state can further activate a variety of downstream signaling pathways. Based on this working principle, a fluorescence energy resonance transfer microscopy imaging method (Ishikawa-Ankerhold H C et al., Molecules. 2012 April; 17(4):4047-132.) is used to detect corresponding phosphorylation performance of the detection signaling domain, the state change of the molecular conformation of the activation signaling domain and the corresponding activation state performance of the different designs of chimeric antigen receptor artificial molecular machines in response to different external stimulus input signals.

The working model of the molecular machine in FIG. 2B is similar to that in FIG. 2A, including seven parts: the extracellular target molecule binding domain, the extracellular spacer domain, the transmembrane domain, the intracellular spacer domain, the intracellular detection signaling domain, the intracellular linker domain and intracellular activation signaling domain. As shown in FIG. 1H, the extracellular target molecule binding domain can be selected as the recognition binding part of PD-L1 as ligand of the receptor PD-1, the extracellular spacer domain can be selected as the extracellular extension fragment of the transmembrane region of PD-1 (that is, between the extracellular target molecule PD-L1 binding domain and the transmembrane region of PD-1), the transmembrane domain can be selected as the transmembrane region of PD-1, the intracellular spacer domain can selected as an intracellular extension fragment of the transmembrane region of PD-1 (i.e. the intracellular portion of the Truncated PD-1 in FIG. 28), the intracellular detection signaling domain can be selected as immunoreceptor tyrosine-based activation motif fragment (i.e. Sub1˜Sub7 in FIG. 28: CD3ζ ITAM1˜3, CD3ε ITAM, FcRIIA ITAM, FcRγ ITAM, DAP12 ITAM) of CD3ζ, CD3ε, FcRIIA, FcRγ, DAP12 and other molecules, the intracellular activation signaling domain can be selected as tyrosine kinase of SYK/ZAP70 family member and the like, and the intracellular linker domain used to connect the intracellular detection signaling domain and the intracellular activation signaling domain can be selected as flexible linker peptide fragment (i.e. the linker peptides with different lengths in FIG. 28: SL, ML, LL1, LL2), see FIG. 1H and FIG. 28.

Further, based on the characteristics of the molecular conformation of SYK/ZAP70 family member, in its inactive state, SYK or ZAP70 can be in an auto-inhibited molecular conformation state. When the target molecule of the target cell is present, the extracellular target molecule binding domain of the chimeric antigen receptor molecular machine on the surface of the immune cell will identify and bind to the target molecule, thereby providing target molecule recognition binding signal input through such recognition and binding, and then the molecular conformation of the intracellular part undergoes a similar change to that described in FIG. 2A above, and finally the intracellular activation signaling domain obtains sufficient release and activation based on the molecular conformational change of the chimeric antigen receptor molecular machine in response to the upstream target molecule recognition binding signal input, and the activation signaling domain in the activation state can further activate a variety of downstream signaling pathways, such that immune cells modified by the chimeric antigen receptor perform specific functions on target cells, such as the killing function of immune T cells or phagocytes against cancer cells. Therefore, FIG. 2B is a schematic diagram of the signal activation of the chimeric antigen receptor artificial molecular machine under the condition that the target molecule recognition binding signal is input. Similarly, similar to the above-mentioned FIG. 2A, based on this working principle, the fluorescence energy resonance transfer microscopy imaging method is used to detect corresponding phosphorylation performance of the detection signaling domain, the state change of the molecular conformation of the activation signaling domain and the corresponding activation state performance of the different designs of chimeric antigen receptor artificial molecular machines in response to different external stimulus input signals.

In summary, the microscopy imaging method is used to detect the response to different external stimulus input signals of the different designs of chimeric antigen receptor artificial molecular machines. In addition, for the convenience of quantitative analysis, the imaging reading index is used to represent the degree of the response ability of chimeric antigen receptor to the stimulus signal and the degree of the release and activation of the self-activating element of the chimeric antigen receptor based on the molecular conformational change, which is simultaneously triggered in response to the stimulus signal.

Proteins C #9 and C #10 are purified from transfected 293T cells using chromatographic purification technologies and protein dialysis at 4° C., then the purified molecular machine proteins are dissolved in kinase buffer solution (50 mM Tris hydrochloride solution with pH at about 8, 100 mM NaCl, 10 mM MgCl₂, 2 mM dithiothreitol) at a concentration of 50 nM, and 1 mM ATP and 100 nM non-acceptor protein tyrosine kinase Lck protein at the activation state, that are provided as the substrate required for phosphorylation, are added therein. Here, Lck protein can provide phosphorylation signal input for the immunoreceptor tyrosine-based activation motif. The optical signals before and after the addition of ATP and Lck are detected and quantitatively analyzed, as shown in FIG. 2A for the signal activation pattern of the artificial molecular machine.

The C #9(+) group (n=3) of the histogram in FIG. 5 proves the followings: the intracellular detection signaling domain Sub1 included in the chimeric antigen receptor C #9 version in the experimental group has very good response ability to protein tyrosine phosphorylation signal (the average is 0.8 in C #9(+) group), the corresponding highly obvious molecular conformational change of the chimeric antigen receptor C #9 version, and very sufficient release and activation of the self-activating element (i.e., the intracellular activation signaling domain ZAP70) based on such molecular conformational change. In addition, the C #10(+) group (n=3) proves the followings: under the condition that the self-detection element is disabled (inactive mutant Sub1FF), comparing with the chimeric antigen receptor C #9 version in the experimental group, the chimeric antigen receptor C #10 version in the control group has significantly different and much weaker response to protein tyrosine phosphorylation signal after statistical analysis (average of the C #10(+) group is 0.078), thereby proving the importance of the intracellular detection signaling domain included in the chimeric antigen receptor C #9 version in terms of excellent response ability to protein tyrosine phosphorylation signal, and that the chimeric antigen receptor C #9 version has excellent specificity in response to protein tyrosine phosphorylation signal. Please refer to FIG. 28 and related description according to the present application for the information of each component included in the chimeric antigen receptor C #9 version and C #10 version. Here, the non-receptor-type protein tyrosine kinase Lck can promote the activation of protein tyrosine phosphorylation signal and play a role in providing input of specific protein tyrosine phosphorylation signal.

Liposome transfection is used to achieve the expression of different molecular machine proteins in mammalian cells such as human cells and mouse cells, and then fluorescence microscopy imaging is used to detect and characterize the performance of different artificial molecular machines in human HeLa cells and mouse embryonic fibroblasts MEFs in response to a variety of different external stimulus input signals.

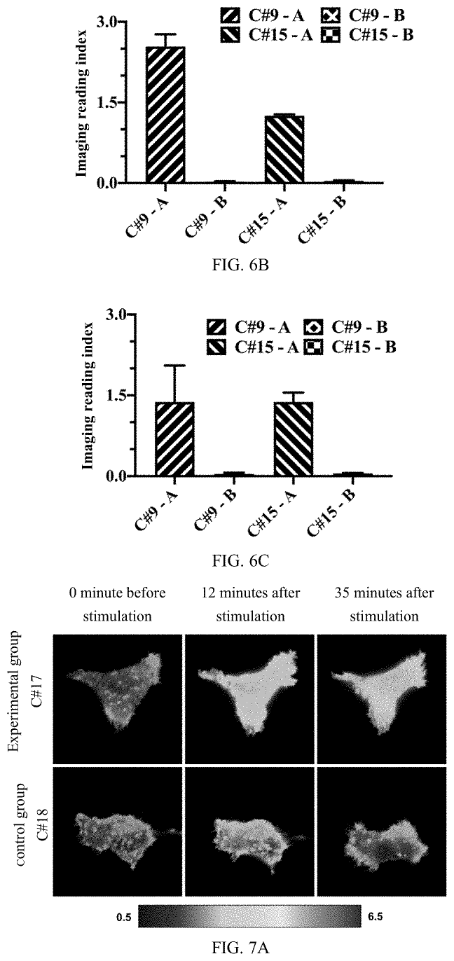

The histogram of FIG. 6A proves the followings: the intracellular detection signaling domains Sub1 and Sub4 included in the artificial molecular machines C #9 version and C #15 version in human HeLa cells in the experimental group have very good response ability to protein tyrosine phosphorylation signal, the corresponding highly obvious molecular conformational change of the artificial molecular machines C #9 version and C #15 version, and very sufficient release and activation of the self-activating element (i.e., the intracellular activation signaling domain ZAP70 and SYK) based on such molecular conformational change; and the artificial molecular machines C #9 version and C #15 version perform significantly better than the artificial molecular machines C #11 version and C #13 version in the experimental group. In addition, under the condition that the self-activating element is disabled (inactive mutant Sub1FF˜Sub4FF), comparing with corresponding artificial molecular machines C #9 version, C #11 version, C #13 version and C #15 version in the experimental group, the artificial molecular machines C #10 version, C #12 version, C #14 version and C #16 version in the control group have significantly different and much weaker (nearly zero) response ability to protein tyrosine phosphorylation signal after statistical analysis, thereby proving the importance of the intracellular detection signaling domains (Sub1˜Sub4) included in the artificial molecular machines C #9 version, C #11 version, C #13 version and C #15 version in terms of excellent response ability to protein tyrosine phosphorylation signal, and that the artificial molecular machines C #9 version (Sub1) and C #15 version (Sub4) have significantly different and better response to the protein tyrosine phosphorylation signal and sensitivity after statistical analysis than the artificial molecular machines C #11 version (Sub2) and C #13 version (Sub3). Please refer to FIG. 28 and related description according to the present application for the information of each component included in the artificial molecular machines C #9 version to C #16 version. Here, the sodium pervanadate (20 uM) as tyrosine phosphatase inhibitor can inhibit the dephosphorylation of intracellular protein, thereby promoting the activation of protein tyrosine phosphorylation signal and playing a role in providing input of protein tyrosine phosphorylation signal.

FIG. 6B shows performance result histogram of different artificial molecular machines in human HeLa cell (the data therein is shown as mean±standard deviation, for each of C #9-A group and C #15-A group, n=5; for each of C #9-B group and C #15-B group, n=3) under the condition A where 20 uM sodium pervanadate as tyrosine phosphatase inhibitor activates the protein tyrosine phosphorylation signal or the condition B where 50 ng/mL epidermal growth factor (EGF) activates the signal, wherein, the imaging reading index represents the degree of the response ability of the artificial molecular machine to the stimulus signal after quantification of data and the degree of the release and activation of the self-activating element of the artificial molecular machine based on the molecular conformational change, which is simultaneously triggered in response to the stimulus signal. Furthermore, the histogram of FIG. 6B proves the followings: the intracellular detection signaling domains Sub1 and Sub4 included in the artificial molecular machines C #9 version and C #15 version in human HeLa cells in the experimental group have very good response ability to protein tyrosine phosphorylation signal, the corresponding highly obvious molecular conformational change of the artificial molecular machines C #9 version and C #15 version, and very sufficient release and activation of the self-activating element (i.e., the intracellular activation signaling domain ZAP70 and SYK) based on such molecular conformational change. In addition, in the presence of the epidermal growth factor activation signal, the artificial molecular machines C #9 version and C #15 version in the experimental group have significantly different and much weaker (nearly zero) response to such signal after statistical analysis, thereby proving the importance of the intracellular detection signaling domains (Sub1 and Sub4) included in the artificial molecular machines C #9 version and C #15 version in terms of excellent response ability to particular protein tyrosine phosphorylation signal, and guaranteeing that the artificial molecular machines specifically respond to the protein tyrosine phosphorylation signal but will not respond to irrelevant signal input such as the epidermal growth factor activation signal. Please refer to FIG. 28 and related description according to the present application for the information of each component included in the artificial molecular machines C #9 version and C #15 version. Here, the sodium pervanadate as tyrosine phosphatase inhibitor can inhibit the dephosphorylation of intracellular protein, thereby promoting the activation of protein tyrosine phosphorylation signal and playing a role in providing input of protein tyrosine phosphorylation signal; and the epidermal growth factor can bind to the epidermal growth factor receptor on the surface of HeLa cell to provide the epidermal growth factor activation signal which does not participate in the phosphorylation of the immunoreceptor tyrosine-based activation motif and thus cannot be specifically detected by the intracellular detection signaling domains contained in artificial molecular machines C #9 version and C #15 version.

FIG. 6C shows performance result histogram of different artificial molecular machines in mouse embryonic fibroblast (MEF) (for each of C #9-A group, C #9-B group, C #15-A group and C #15-B group, n=5) under the condition A where 20 uM sodium pervanadate as tyrosine phosphatase inhibitor activates the protein tyrosine phosphorylation signal or the condition B where 50 ng/mL platelet-derived growth factor (PDGF) activates the signal, wherein, the imaging reading index represents the degree of the response ability of the artificial molecular machine to the stimulus signal after quantification of data and the degree of the release and activation of the self-activating element of the artificial molecular machine based on the molecular conformational change, which is simultaneously triggered in response to the stimulus signal. Furthermore, the histogram of FIG. 6C proves the followings: the intracellular detection signaling domains Sub1 and Sub4 included in the artificial molecular machines C #9 version and C #15 version in mouse embryonic fibroblasts in the experimental group have very good response ability to protein tyrosine phosphorylation signal, the corresponding highly obvious molecular conformational change of the artificial molecular machines C #9 version and C #15 version, and very sufficient release and activation of the self-activating element (i.e., the intracellular activation signaling domain ZAP70 and SYK) based on such molecular conformational change. In addition, in the presence of the platelet-derived growth factor activation signal, the artificial molecular machines C #9 version and C #15 version in the experimental group have significantly different and much weaker (nearly zero) response to such signal after statistical analysis, thereby proving the importance of the intracellular detection signaling domains (Sub1 and Sub4) included in the artificial molecular machines C #9 version and C #15 version in terms of excellent response ability to protein tyrosine phosphorylation signal, and guaranteeing that the artificial molecular machines specifically respond to particular protein tyrosine phosphorylation signal but will not respond to irrelevant signal input such as the platelet-derived growth factor activation signal. Please refer to FIG. 28 and related description according to the present application for the information of each component included in the artificial molecular machines C #9 version and C #15 version. Here, the sodium pervanadate as tyrosine phosphatase inhibitor can inhibit the dephosphorylation of intracellular protein, thereby promoting the activation of protein tyrosine phosphorylation signal and playing a role in providing input of protein tyrosine phosphorylation signal; and the platelet-derived growth factor can bind to the platelet-derived growth factor receptor on the surface of the mouse embryonic fibroblast to provide the platelet-derived growth factor activation signal which does not participate in the phosphorylation of the immunoreceptor tyrosine-based activation motif and thus cannot be specifically detected by the intracellular detection signaling domains contained in artificial molecular machines C #9 version and C #15 version.

Liposome transfection is used to achieve the expression of different chimeric antigen receptor proteins in human cells, and then the fluorescence microscopy imaging is used to detect and characterize the expression distribution of different immune checkpoint PD-1 fusion-based chimeric antigen receptors in human HeLa cells and the performance of different immune checkpoint PD-1 fusion-based chimeric antigen receptors in human HeLa cells in response to a variety of different external stimulus input signals.

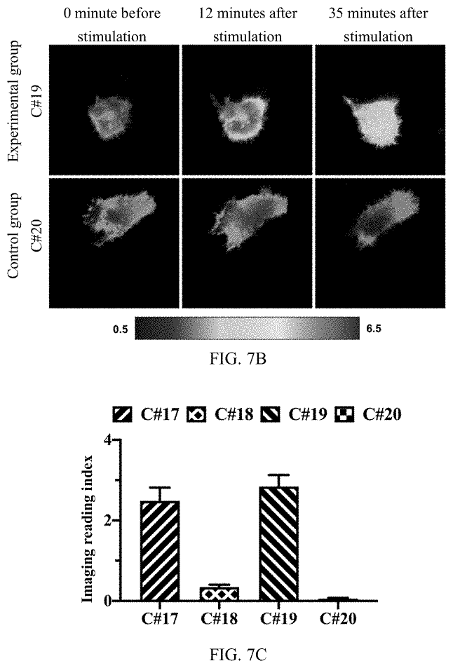

FIG. 7A shows the expression distribution of different immune checkpoint PD-1 fusion-based chimeric antigen receptor artificial molecular machines in human HeLa cell and detection result for the ability to respond to the protein tyrosine phosphorylation signal stimulated by 20 uM sodium pervanadate as the tyrosine phosphatase inhibitor; wherein, the experimental group refers to human HeLa cell modified with the immune checkpoint PD-1 fusion-based chimeric antigen receptor C #17 version according to the present disclosure while the control group refers to Human HeLa cell modified with the immune checkpoint PD-1 fusion-based chimeric antigen receptor C #18 version according to the present disclosure, the color bar heat map under the Figures from left to right represents from low to high response ability of the chimeric antigen receptor to stimulus signal, and from low to high degree of the release and activation of the self-activating element (i.e., the intracellular activation signaling domain) of the chimeric antigen receptor based on the molecular conformational change, which is simultaneously triggered in response to the stimulus signal. First, as shown in FIG. 7A, the PD-1-fused chimeric antigen receptors C #17 version and C #18 version both display the correct membrane-localized expression distribution on the surface of human HeLa cells, without any other wrong protein localization. In addition, the human HeLa cells modified by the C #17 version in the experimental group show rapid and significant response ability to the protein tyrosine phosphorylation signal obtained by stimulus of the sodium pervanadate as the tyrosine phosphatase inhibitor, and show extremely significant response ability to the stimulus signal and the release and activation of its own intracellular activation signaling domain based on molecular conformation change after about half an hour of stimulation; while the human HeLa cells modified by the C #18 version in the control group show significantly weaker response ability to the protein tyrosine phosphorylation signal obtained by stimulus of the sodium pervanadate as the tyrosine phosphatase inhibitor, and could not exhibit an effective response ability to the stimulus signal and the release and activation of its own intracellular activation signaling domain based on molecular conformation change after stimulation. The above results fully prove that the signal activation mode of the artificial molecular machine shown in FIGS. 2A-2B in human cells.

FIG. 7A proves the followings: the intracellular detection signaling domain Sub1 included in the chimeric antigen receptor C #17 version in human HeLa cells has excellent response ability to protein tyrosine phosphorylation signal, the corresponding highly obvious molecular conformational change of the chimeric antigen receptor C #17 version, and very sufficient release and activation of the self-activating element (i.e., the intracellular activation signaling domain ZAP70) based on such molecular conformational change. In addition, under the condition that the self-activating element is disabled (inactive mutant Sub1FF), comparing with the artificial molecular machines C #17 version in the experimental group, the artificial molecular machine C #18 version in the control group has much weaker (nearly zero) response ability to the protein tyrosine phosphorylation signal, thereby proving the importance and specificity of the intracellular detection signaling domain (Sub1) included in the artificial molecular machine C #17 version in terms of excellent response ability to protein tyrosine phosphorylation signal. Please refer to FIG. 28 and related description according to the present application for the information of each component included in the immune checkpoint PD-1 fusion-based chimeric antigen receptor C #17 version and C #18 version. Here, the sodium pervanadate as tyrosine phosphatase inhibitor can inhibit the dephosphorylation of intracellular protein, thereby promoting the activation of protein tyrosine phosphorylation signal and playing a role in providing input of protein tyrosine phosphorylation signal.

FIG. 7B shows the expression distribution of different immune checkpoint PD-1 fusion-based chimeric antigen receptor artificial molecular machines in human HeLa cells and detection result for the ability to respond to protein tyrosine phosphorylation signal stimulated by 20 uM sodium pervanadate as the tyrosine phosphatase inhibitor; wherein, the experimental group refers to human HeLa cell modified with the immune checkpoint PD-1 fusion-based chimeric antigen receptor C #19 version according to the present disclosure while the control group refers to human HeLa cell modified with the immune checkpoint PD-1 fusion-based chimeric antigen receptor C #20 version according to the present disclosure, the color bar heat map under the Figures from left to right represents from low to high response ability of the chimeric antigen receptor to stimulus signal, and from low to high degree of the release and activation of the self-activating element (i.e., the intracellular activation signaling domain) of the chimeric antigen receptor based on the molecular conformational change, which is simultaneously triggered in response to the stimulus signal. First, as shown in FIG. 7B, the PD-1-fused chimeric antigen receptors C #19 version and C #20 version both display the correct membrane-localized expression distribution on the surface of human HeLa cells, without any other wrong protein localization. In addition, the human HeLa cells modified by the C #19 version in the experimental group show rapid and significant response ability to the protein tyrosine phosphorylation signal obtained by stimulus of the sodium pervanadate as the tyrosine phosphatase inhibitor, and show extremely significant response ability to the stimulus signal and the release and activation of its own intracellular activation signaling domain based on molecular conformation change after about half an hour of stimulation; while the human HeLa cells modified by the C #20 version in the control group show significantly weaker (nearly zero) response ability to the protein tyrosine phosphorylation signal obtained by stimulus of the sodium pervanadate as the tyrosine phosphatase inhibitor, and could not exhibit an effective response ability to the stimulus signal and the release and activation of its own intracellular activation signaling domain based on molecular conformation change after stimulation. The above results fully prove that the signal activation mode of the artificial molecular machine shown in FIGS. 2A-2B in human cells.

FIG. 7B proves the followings: the intracellular detection signaling domain Sub1 included in the chimeric antigen receptor C #19 version in human HeLa cells has excellent response ability to protein tyrosine phosphorylation signal, the corresponding highly obvious molecular conformational change of the chimeric antigen receptor C #19 version, and very sufficient and significant release and activation of the self-activating element (i.e., the intracellular activation signaling domain) based on such molecular conformational change. In addition, under the condition that the self-activating element is disabled (inactive mutant Sub1 FF), comparing with the artificial molecular machines C #19 version in the experimental group, the artificial molecular machine C #20 version in the control group has much weaker (nearly zero) response ability to the protein tyrosine phosphorylation signal, thereby proving the importance and specificity of the intracellular detection signaling domain (Sub1) included in the artificial molecular machine C #19 version in terms of excellent response ability to the protein tyrosine phosphorylation signal. Please refer to FIG. 28 and related description according to the present application for the information of each component included in the immune checkpoint PD-1 fusion-based chimeric antigen receptor C #19 version and C #20 version. Here, the sodium pervanadate as tyrosine phosphatase inhibitor can inhibit the dephosphorylation of intracellular protein, thereby promoting the activation of protein tyrosine phosphorylation signal and playing a role in providing input of protein tyrosine phosphorylation signal.

FIG. 7C shows performance result histogram of different immune checkpoint PD-1 fusion-based chimeric antigen receptor artificial molecular machines in human HeLa cell (the data therein is shown as mean±standard deviation, for each of C #17 group to C #20 group, n=10) under the condition that the sodium pervanadate as tyrosine phosphatase inhibitor activates the protein tyrosine phosphorylation signal, wherein, the imaging reading index represents the degree of the response ability of chimeric antigen receptor to the stimulus signal after quantification of data and the degree of the release and activation of the self-activating element of the chimeric antigen receptor based on the molecular conformational change, which is simultaneously triggered in response to the stimulus signal. Furthermore, the histogram of FIG. 7C proves the followings: the intracellular detection signaling domain Sub1 included in the chimeric antigen receptor C #19 version in human HeLa cells in the experimental group has very good response ability to protein tyrosine phosphorylation signal (the average of C #19 group is 2.841), the corresponding highly obvious molecular conformational change of the chimeric antigen receptor C #19 version, very sufficient and significant release and activation of the self-activating element (i.e. the intracellular activation signaling domain) based on such molecular conformational change, and the chimeric antigen receptor C #19 version performs significantly different and better than the chimeric antigen receptor C #17 version in the experimental group (the average of C #17 group is 2.484) after statistical analysis. In addition, under the condition that the self-activating element is disabled (inactive mutant Sub1FF), comparing with the chimeric antigen receptor C #18 version in the control group, the chimeric antigen receptor C #20 version in the control group has significantly different and much weaker response ability to the protein tyrosine phosphorylation signal after statistical analysis (the average of C #20 group is 0.0549 and the average of C #18 group is 0.344), thereby proving the importance of the intracellular detection signaling domain included in the chimeric antigen receptors C #19 version and C #17 version in terms of excellent response ability to protein tyrosine phosphorylation signal, and the chimeric antigen receptor C #19 version has significantly better performance than the chimeric antigen receptor C #17 version in terms of the response specificity to protein tyrosine phosphorylation signal, thereby proving that the intracellular spacer domain used in the C #19 version has better functional performance than the intracellular spacer domain in the C #17 version.

DNA electroporation transfection is used to achieve the expression of different chimeric antigen receptor proteins in human cells, and then the fluorescence microscopy imaging is used to detect and characterize the expression distribution of different immune checkpoint PD-1 fusion-based chimeric antigen receptors in human Jurkat E6-1 T lymphocytes and the performance of different immune checkpoint PD-1 fusion-based chimeric antigen receptors in human Jurkat E6-1 T lymphocytes in response to a variety of different external stimulus input signals.

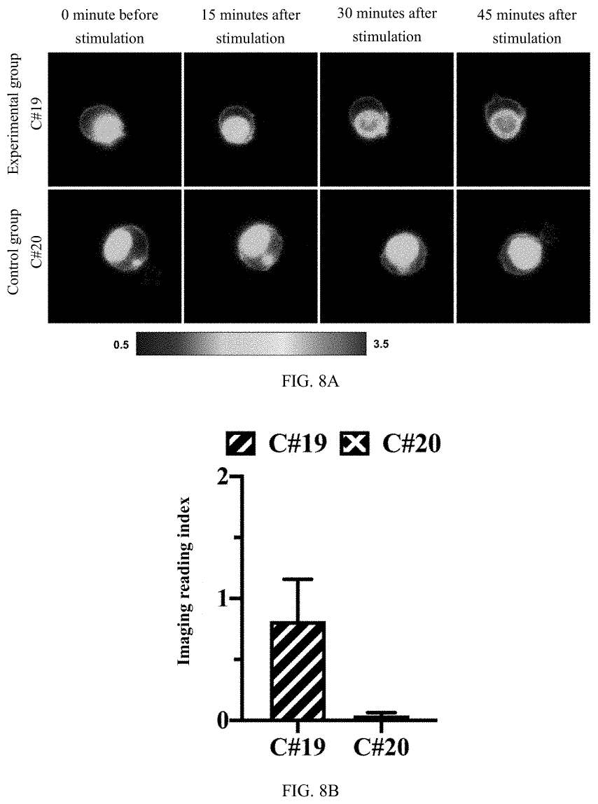

FIG. 8A shows the expression distribution of different immune checkpoint PD-1 fusion-based chimeric antigen receptor artificial molecular machines in human Jurkat E6-1 cell and detection result for the ability to respond to protein tyrosine phosphorylation signal stimulated by 20 uM sodium pervanadate as the tyrosine phosphatase inhibitor; wherein, the experimental group refers to human Jurkat E6-1 cell modified with the immune checkpoint PD-1 fusion-based chimeric antigen receptor C #19 version according to the present disclosure while the control group refers to human Jurkat E6-1 cell modified with the immune checkpoint PD-1 fusion-based chimeric antigen receptor C #20 version according to the present disclosure, the color bar heat map under the Figures from left to right represents from low to high response ability of the chimeric antigen receptor to stimulus signal, and from low to high degree of the release and activation of the self-activating element (i.e., the intracellular activation signaling domain) of the chimeric antigen receptor based on the molecular conformational change, which is simultaneously triggered in response to the stimulus signal. First, as shown in FIG. 8A, the PD-1-fused chimeric antigen receptors C #19 version and C #20 version both display the correct membrane-localized expression distribution on the surface of human Jurkat E6-1 T lymphocytes, without any other wrong protein localization. In addition, the human Jurkat E6-1 T lymphocytes modified by the C #19 version in the experimental group show rapid and significant response ability to the protein tyrosine phosphorylation signal obtained by stimulus of the sodium pervanadate as the tyrosine phosphatase inhibitor, and show extremely significant response ability to the stimulus signal and the release and activation of its own intracellular activation signaling domain based on molecular conformation change after about half an hour of stimulation; while the human Jurkat E6-1 T lymphocytes modified by the C #20 version in the control group show significantly weaker (nearly zero) response ability to the protein tyrosine phosphorylation signal obtained by stimulus of the sodium pervanadate as the tyrosine phosphatase inhibitor, and could not exhibit an effective response ability to the stimulus signal and the release and activation of its own intracellular activation signaling domain based on molecular conformation change after stimulation. The above results fully prove that the signal activation mode of the artificial molecular machine shown in FIGS. 2A-2B in human lymphocytes.

FIG. 8A proves the followings: the intracellular detection signaling domain Sub1 included in the chimeric antigen receptor C #19 version in human lymphocytes has excellent response ability to protein tyrosine phosphorylation signal, the corresponding highly obvious molecular conformational change of the chimeric antigen receptor C #19 version, and very sufficient and significant release and activation of the self-activating element (i.e., the intracellular activation signaling domain) based on such molecular conformational change. In addition, under the condition that the self-activating element is disabled (inactive mutant Sub1 FF), comparing with the artificial molecular machines C #19 version in the experimental group, the artificial molecular machine C #20 version in the control group has much weaker (nearly zero) response ability to the protein tyrosine phosphorylation signal, thereby proving the importance and specificity of the intracellular detection signaling domain (Sub1) included in the artificial molecular machine C #19 version in terms of excellent response ability to the protein tyrosine phosphorylation signal. Please refer to FIG. 28 and related description according to the present application for the information of each component included in the immune checkpoint PD-1 fusion-based chimeric antigen receptor C #19 version and C #20 version. Here, the sodium pervanadate as tyrosine phosphatase inhibitor can inhibit the dephosphorylation of intracellular protein, thereby promoting the activation of protein tyrosine phosphorylation signal and playing a role in providing input of protein tyrosine phosphorylation signal.

FIG. 8B shows performance result histogram of different immune checkpoint PD-1 fusion-based chimeric antigen receptor artificial molecular machines in human Jurkat E6-1 cell (the data therein is shown as mean±standard deviation, for each of C #19 group and C #20 group, n=10) under the condition that the sodium pervanadate as tyrosine phosphatase inhibitor activates the protein tyrosine phosphorylation signal, wherein, the imaging reading index represents the degree of the response ability of chimeric antigen receptor to the stimulus signal after quantification of data and the degree of the release and activation of the self-activating element of the chimeric antigen receptor based on the molecular conformational change, which is simultaneously triggered in response to the stimulus signal. Furthermore, the histogram of FIG. 8B proves the followings: the intracellular detection signaling domain Sub1 included in the chimeric antigen receptor C #19 version in human lymphocytes in the experimental group has very good response ability to protein tyrosine phosphorylation signal (the average of the C #19 group is 0.815), the corresponding highly obvious molecular conformational change of the chimeric antigen receptor C #19 version, and very sufficient and significant release and activation of the self-activating element (i.e., the intracellular activation signaling domain) based on such molecular conformational change. In addition, under the condition that the self-activating element is disabled (inactive mutant Sub1FF), comparing with the chimeric antigen receptor C #19 version in the experimental group, the chimeric antigen receptor C #20 version in the control group has significantly different and much weaker response ability to the protein tyrosine phosphorylation signal after statistical analysis (the average of C #20 group is 0.0409), thereby proving the importance of the intracellular detection signaling domain included in the chimeric antigen receptor C #19 version in terms of excellent response ability to protein tyrosine phosphorylation signal, and the chimeric antigen receptor C #19 version has excellent response specificity to protein tyrosine phosphorylation signal, thereby proving that the intracellular spacer domain used in the C #19 version has better functional performance.

Liposome transfection or DNA electroporation transfection is used to achieve the expression of different chimeric antigen receptor proteins in human cells, and then the fluorescence microscopy imaging is used to detect and characterize the expression distribution of different immune checkpoint PD-1 fusion-based chimeric antigen receptors in human HeLa cells and human Jurkat E6-1 T lymphocytes and the performance of different immune checkpoint PD-1 fusion-based chimeric antigen receptors in human HeLa cells and human Jurkat E6-1 T lymphocytes in response to physiologically specific human PD-L1 signal input, wherein the physiologically specific human PD-L1 signal used refers to human PD-L1-coated bead particles.

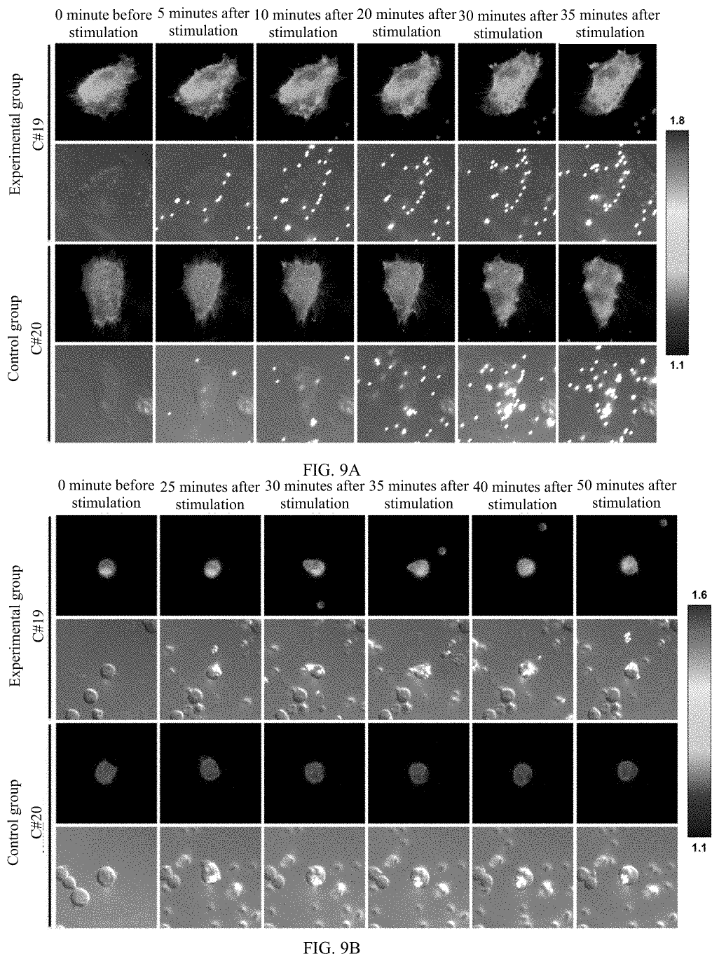

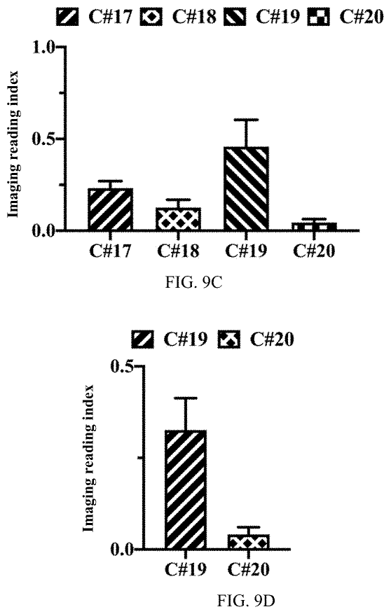

FIG. 9A shows the expression distribution of different immune checkpoint PD-1 fusion-based chimeric antigen receptor artificial molecular machines in human HeLa cell and detection result in response ability to human PD-L1 signal stimulated by the human PD-L1-modified microspheres; wherein, the experimental group refers to human HeLa cell modified with the immune checkpoint PD-1 fusion-based chimeric antigen receptor C #19 version according to the present disclosure while the control group refers to human HeLa cell modified with the immune checkpoint PD-1 fusion-based chimeric antigen receptor C #20 version according to the present disclosure, the color bar heat map on the right of Figures from bottom to up represents from low to high response ability of the chimeric antigen receptor to stimulus signal, and from low to high degree of the release and activation of the self-activating element (i.e. intracellular activation signaling domain ZAP70) of the chimeric antigen receptor based on the molecular conformational change, which is simultaneously triggered in response to the stimulus signal; and the phase-contrast imaging experimental figures provide the image information of the interaction between cell and microsphere.

First, as shown in FIG. 9A, the PD-1-fused chimeric antigen receptors C #19 version and C #20 version both display the correct membrane-localized expression distribution on the surface of human HeLa cells, without any other wrong protein localization. In addition, the human HeLa cells modified by the C #19 version in the experimental group show rapid and significant response ability to the signal stimulated by the human PD-L1-modified microspheres, and show extremely significant response ability to the stimulus signal and the release and activation of its own intracellular activation signaling domain based on molecular conformation change since about 10 minutes after stimulation, and the response to the stimulation signal of human PD-L1 modified microspheres has highly specific spatial characteristics, that is, the response ability is only locally displayed at the position where the cells interact with the microspheres in the phase-contrast imaging experimental figures; while the human HeLa cells modified by the C #20 version in the control group show significantly weaker response ability to the signal stimulated by the human PD-L1-modified microspheres, and could not exhibit an effective response ability to the stimulus signal and the release and activation of its own intracellular activation signaling domain based on molecular conformation change after stimulation. The above results fully prove that the signal activation mode of the artificial molecular machine shown in FIG. 2B in human cells.

FIG. 9A proves the followings: the intracellular detection signaling domain Sub1 included in the chimeric antigen receptor C #19 version in human HeLa cells has excellent response ability to human PD-L1 signal, the corresponding highly obvious molecular conformational change of the chimeric antigen receptor C #19 version, and very sufficient release and activation of the self-activating element (i.e. the intracellular activation signaling domain ZAP70) based on such molecular conformational change. In addition, under the condition that the self-activating element is disabled (inactive mutant Sub1FF), comparing with the artificial molecular machines C #19 version in the experimental group, the artificial molecular machine C #20 version in the control group has much weaker response ability to the human PD-L1 signal, thereby proving the importance and specificity of the intracellular detection signaling domain (Sub1) included in the artificial molecular machine C #19 version in terms of excellent response ability to human PD-L1 signal. Please refer to FIG. 28 and related description according to the present application for the information of each component included in the immune checkpoint PD-1 fusion-based chimeric antigen receptor C #19 version and C #20 version. Here, the human PD-L1-modified microspheres play a role of providing human PD-L1 signal input.

FIG. 9B shows the expression distribution of different immune checkpoint PD-1 fusion-based chimeric antigen receptor artificial molecular machines in human Jurkat E6-1 cell and detection result in response to human PD-L1 signal stimulated by the human PD-L1-modified microspheres; wherein, the experimental group refers to human Jurkat E6-1 T lymphocyte modified with the immune checkpoint PD-1 fusion-based chimeric antigen receptor C #19 version according to the present disclosure while the control group refers to human Jurkat E6-1 T lymphocyte modified with the immune checkpoint PD-1 fusion-based chimeric antigen receptor C #20 version according to the present disclosure, the color bar heat map on the right of Figures from bottom to up represents from low to high response ability of the chimeric antigen receptor to stimulus signal, and from low to high degree of the release and activation of the self-activating element (i.e. the intracellular activation signaling domain ZAP70) of the chimeric antigen receptor based on the molecular conformational change, which is simultaneously triggered in response to the stimulus signal; and the phase-contrast imaging experimental figures provide the image information of the interaction between cell and microsphere.

First, as shown in FIG. 9B, the PD-1-fused chimeric antigen receptors C #19 version and C #20 version both display the correct membrane-localized expression distribution on the surface of Jurkat E6-1 T lymphocytes, without any other wrong protein localization. In addition, the human Jurkat E6-1 T lymphocytes modified by the C #19 version in the experimental group show rapid and significant response ability to the signal stimulated by the human PD-L1-modified microspheres, and show extremely significant response ability to the stimulus signal and the release and activation of its own intracellular activation signaling domain based on molecular conformation change since about 25 minutes after stimulation, and the response to the stimulation signal of human PD-L1 modified microspheres has highly specific spatial characteristics, that is, the response ability is only locally displayed at the position where the cells interact with the microspheres in the phase-contrast imaging experimental figures; while the human Jurkat E6-1 T lymphocytes modified by the C #20 version in the control group show nearly zero response ability to the signal stimulated by the human PD-L1-modified microspheres, and could not exhibit an effective response ability to the stimulus signal and the release and activation of its own intracellular activation signaling domain based on molecular conformation change after stimulation. The above results fully prove that the signal activation mode of the artificial molecular machine shown in FIG. 2B in human lymphocytes.

FIG. 9B proves the followings: the intracellular detection signaling domain Sub1 included in the chimeric antigen receptor C #19 version in human Jurkat E6-1 T lymphocytes has excellent response ability to human PD-L1 signal, the corresponding highly obvious molecular conformational change of the chimeric antigen receptor C #19 version, and very sufficient and significant release and activation of the self-activating element (i.e. the intracellular activation signaling domain ZAP70) based on such molecular conformational change. In addition, under the condition that the self-activating element is disabled (inactive mutant Sub1FF), comparing with the artificial molecular machines C #19 version in the experimental group, the artificial molecular machine C #20 version in the control group has much weaker response ability to the human PD-L1 signal, thereby proving the importance and specificity of the intracellular detection signaling domain (Sub1) included in the artificial molecular machine C #19 version in terms of excellent response ability to human PD-L1 signal. Please refer to FIG. 28 and related description according to the present application for the information of each component included in the immune checkpoint PD-1 fusion-based chimeric antigen receptor C #19 version and C #20 version. Here, the human PD-L1-modified microspheres play a role of providing human PD-L1 signal input.

FIG. 9C shows performance result histogram of different immune checkpoint PD-1 fusion-based chimeric antigen receptor artificial molecular machines in human HeLa cell (the data therein is shown as mean±standard deviation, for each of C #17 group to C #20 group, n=10) under the condition that the signal is stimulated by the human PD-L1-modified microsphere, wherein, the imaging reading index represents the degree of the response ability of chimeric antigen receptor to the stimulus signal after quantification of data and the degree of the release and activation of the self-activating element of the chimeric antigen receptor based on the molecular conformational change, which is simultaneously triggered in response to the stimulus signal. Furthermore, the histogram of FIG. 9C proves the followings: the intracellular detection signaling domain Sub1 included in the chimeric antigen receptor C #19 version in human HeLa cells in the experimental group has very good response ability to protein tyrosine phosphorylation signal (the average of the C #19 group is 0.458), the corresponding highly obvious molecular conformational change of the chimeric antigen receptor C #19 version, and very sufficient and significant release and activation of the self-activating element (i.e., the intracellular activation signaling domain ZAP70) based on such molecular conformational change, and the chimeric antigen receptor C #19 version performs significantly different and better than the chimeric antigen receptor C #17 version in the experimental group (the average of C #17 group is 0.232) after statistical analysis. In addition, under the condition that the self-activating element is disabled (inactive mutant Sub1FF), comparing with the chimeric antigen receptor C #18 version in the control group, the chimeric antigen receptor C #20 version in the control group has significantly different and much weaker response ability to the protein tyrosine phosphorylation signal after statistical analysis (the average of C #20 group is 0.0445 and the average of C #18 group is 0.127), thereby proving the importance of the intracellular detection signaling domain included in the chimeric antigen receptors C #19 version and C #17 version in terms of excellent response ability to human PD-L1 signal, and the chimeric antigen receptor C #19 version has significantly better performance than the chimeric antigen receptor C #17 version in terms of the response specificity to human PD-L1 signal, thereby proving that the intracellular spacer domain used in the C #19 version has better functional performance than the intracellular spacer domain in the C #17 version.

FIG. 9D shows performance result histogram of different immune checkpoint PD-1 fusion-based chimeric antigen receptor artificial molecular machines in human Jurkat E6-1 T lymphocyte (the data therein is shown as mean±standard deviation, for each of C #19 group and C #20 group, n=10) under the condition that the signal is stimulated by the human PD-L1-modified microsphere, wherein, the imaging reading index represents the degree of the response ability of chimeric antigen receptor to the stimulus signal after quantification of data and the degree of the release and activation of the self-activating element of the chimeric antigen receptor based on the molecular conformational change, which is simultaneously triggered in response to the stimulus signal. Furthermore, the histogram of FIG. 9C proves the followings: the intracellular detection signaling domain Sub1 included in the chimeric antigen receptor C #19 version in human Jurkat E6-1 T lymphocytes in the experimental group has very good response ability to human PD-L1 signal (the average of the C #19 group is 0.326), the corresponding highly obvious molecular conformational change of the chimeric antigen receptor C #19 version, and very sufficient and significant release and activation of the self-activating element (i.e., the intracellular activation signaling domain ZAP70) based on such molecular conformational change. In addition, under the condition that the self-activating element is disabled (inactive mutant Sub1FF), comparing with the chimeric antigen receptor C #19 version in the experimental group, the chimeric antigen receptor C #20 version in the control group has significantly different and much weaker (nearly zero) response ability to the human PD-L1 signal (the average of the C #20 group is 0.0412), thereby proving the importance of the intracellular detection signaling domain included in the chimeric antigen receptor C #19 version in terms of excellent response ability to the human PD-L1 signal and the specificity of the chimeric antigen receptor C #19 version in response to the human PD-L1 signal, further proving that the intracellular spacer domain used in the C #19 version has better functional performance.

DNA electroporation transfection is used to achieve the expression of different chimeric antigen receptor proteins in human lymphocytes, and then the Jurkat E6-1 T lymphocytes modified with the immune checkpoint PD-1 fusion-based chimeric antigen receptor artificial molecular machine and PD-L1 positive human breast cancer cell MDA-MB-231 pretreated with interferon-γ are co-cultured in a carbon dioxide cell incubator for at least 24 hours. MDA-MB-231 cells in cell culture dishes are pretreated with 25 ng/mL human interferon-γ for 24 hours before starting co-culture experiments. After 1 day, 2×10⁵˜5×10⁵ MDA-MB-231 cells pretreated with human interferon-γ are inoculated into one well of a 12-well plate culture dish and the same number (i.e. 2×10⁵˜5×10⁵) of Jurkat E6-1 cells modified with the chimeric antigen receptor are added therein to perform co-culture. After 24 hours of co-culture, the modified Jurkat E6-1 T lymphocytes are collected and are subjected to antibody staining and signal detection of flow cytometry. The detected signal is CD69 which is an early activation molecule on the surface of T lymphocytes (Simms P E et al., 1996 May 1; 3(3): 301-4.) and can directly reflect level of immune activation of T lymphocytes under the condition of co-culture with cancer cells. According to the detection level of CD69, the ability of the intracellular activation signaling domain of the chimeric antigen receptor protein in the modified human lymphocytes to activate the lymphocytes under the condition that the corresponding target cell PD-L1 molecule signal is input can be directly characterized. This indicator is used as direct measure of the response effect generated by the binding of the immune checkpoint PD-1 fusion-based chimeric antigen receptor to the target molecule PD-L1. The intracellular activation signaling domain can transmit signals to the interior of the host cells to the downstream and activate the effector function of host cells and the like.

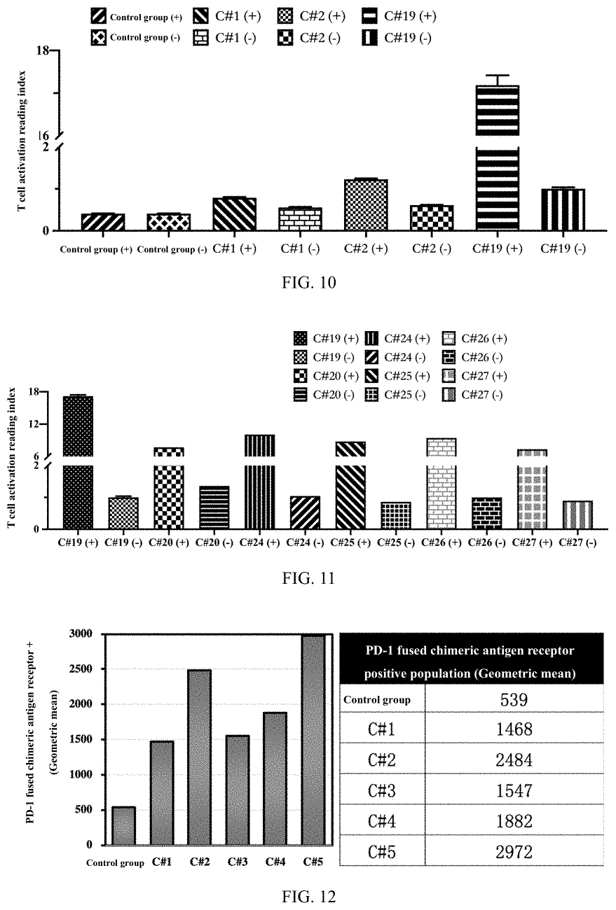

FIG. 10 shows histogram of different T cell activation level performances under the condition that Jurkat E6-1 cell modified with the immune checkpoint PD-1 fusion-based chimeric antigen receptor artificial molecular machine and PD-L1 high-expressing human breast cancer cell MDA-MB-231 pretreated with interferon-γ are co-cultured (the data therein is shown as mean±standard deviation, for C #19 (+) group, n=4, for other groups, n=6, (+) represents the condition where the Jurket E6-1 cell is co-cultured with human breast cancer cell pretreated with interferon-γ, (−) represents the condition where the Jurket E6-1 cell is cultured alone, and the T cell activation read index represents relative expression level of the activating molecule CD69 on the surface of T lymphocyte.

The histogram of FIG. 10 proves the followings: T cells modified with the chimeric antigen receptor C #19 version have excellent T cell activation level when co-cultured with PD-L1-positive human cancer cells (the average of C #19 (+) group is 17.19), while the T cells in other experimental groups and control groups could not effectively show the T cell activation level when co-cultured with PD-L1-positive human cancer cells and show significant difference from T cell activation level in the C #19 (+) group after statistical analysis; wherein, T cells modified with chimeric antigen receptor C #19 version in the experimental group C #19(−) show significantly different and much weaker T cell activation level (the average of C #19(−) group is 1.003) after statistical analysis in the absence of PD-L1-positive human cancer cells providing PD-L1 signal input, thereby proving that the chimeric antigen receptor C #19 version has excellent response specificity to PD-L1-positive human cancer cells. On the other hand, none of the control group (+), C #1 group (+), and C #2 group (+) could effectively show T cell activation, thereby proving that the importance of the intracellular signaling domain of the chimeric antigen receptor C #19 version, especially the intracellular activation signaling thereof, for specific T cell activation when modified T cells face PD-L1-positive cancer cells; wherein, the Jurket E6-1 cells in the control group (+) and the control group (−) are all unmodified wild-type Jurket E6-1 cells, and the T cell activation read index is the relative expression level of the activation molecule CD69 on the surface of T lymphocyte. Please refer to FIG. 28 and related description according to the present application for the information of each component included in the immune checkpoint PD-1 fusion-based chimeric antigen receptor C #1 version, C #2 version and C #19 version.

FIG. 11 shows histogram of different T cell activation level performances under the condition that Jurkat E6-1 cell modified with the immune checkpoint PD-1 fusion-based chimeric antigen receptor artificial molecular machine comprising different lengths of intracellular linker domains and PD-L1 high-expressing human breast cancer cell MDA-MB-231 pretreated with interferon-γ are co-cultured (the data therein is shown as mean±standard deviation, for C #19 (+) group, n=4, for C #19 (−) group, n=6, for the average value of other groups, n=1, (+) represents the condition where the Jurket E6-1 cell is co-cultured with human breast cancer cell pretreated with interferon-γ, (−) represents the condition where the Jurket E6-1 cell is cultured alone, and the T cell activation read index represents relative expression level of the activating molecule CD69 on the surface of T lymphocyte.

The histogram of FIG. 11 proves the followings: T cells modified with the chimeric antigen receptors C #19 version, C #24 version and C #26 version have excellent T cell activation level when co-cultured with PD-L1-positive human cancer cells (the average of C #19 (+) group is 17.19, the average of C #24 (+) group is 10.08, and the average of C #26 (+) group is 9.44), while the T cells modified with the chimeric antigen receptors C #20 version, C #25 version and C #27 version have relatively weak T cell activation level when co-cultured with PD-L1-positive human cancer cells (the average of C #20 (+) group is 7.70, the average of C #25 (+) group is 8.78, and the average of C #27 (+) group is 7.36). In addition, T cells modified with corresponding chimeric antigen receptor versions (especially C #19 version, C #24 version and C #26 version) in each experimental group (−) have significantly weak T cell activation level (the average of C #19(−) group is 1.003, the average of C #24(−) group is 1.04 and the average of C #26(−) group is 1.01) in the absence of PD-L1-positive human cancer cells providing PD-L1 signal input, thereby proving that the corresponding chimeric antigen receptor version has excellent response specificity to PD-L1-positive human cancer cells; wherein, the Jurket E6-1 cells in the control group (+) and the control group (−) are all unmodified wild-type Jurket E6-1 cells, and the T cell activation read index is the relative expression level of the activation molecule CD69 on the surface of T lymphocyte. Please refer to FIG. 28 and related description according to the present application for the information of each component included in the immune checkpoint PD-1 fusion-based chimeric antigen receptor C #19 version, C #20 version, and C #24 version to C #27 version.

To sum up, after detecting and characterizing the extracellular and intracellular functional performance of chimeric antigen receptors by different means, it is proved that the immune checkpoint PD-1 fusion-based chimeric antigen receptor artificial molecular machine exhibits the excellent response ability to different stimulus signal inputs shown in FIGS. 2A-2B, especially a highly specific response to human PD-L1 signal input, and the importance of intracellular signaling domains, especially the proved ability of intracellular activation signaling domain stimulating the effector function of the corresponding modified lymphocytes after being released and activated; wherein, the functionality of the C #19 version (i.e. the Truncated PD-1-Sub1-LL2-ZAP70 version) is particularly prominent, which also provides sufficient information for cytotoxic killing experiments and animal tumor model experiments.

### Example 3 Tumor Cytotoxicity Killing Experiment

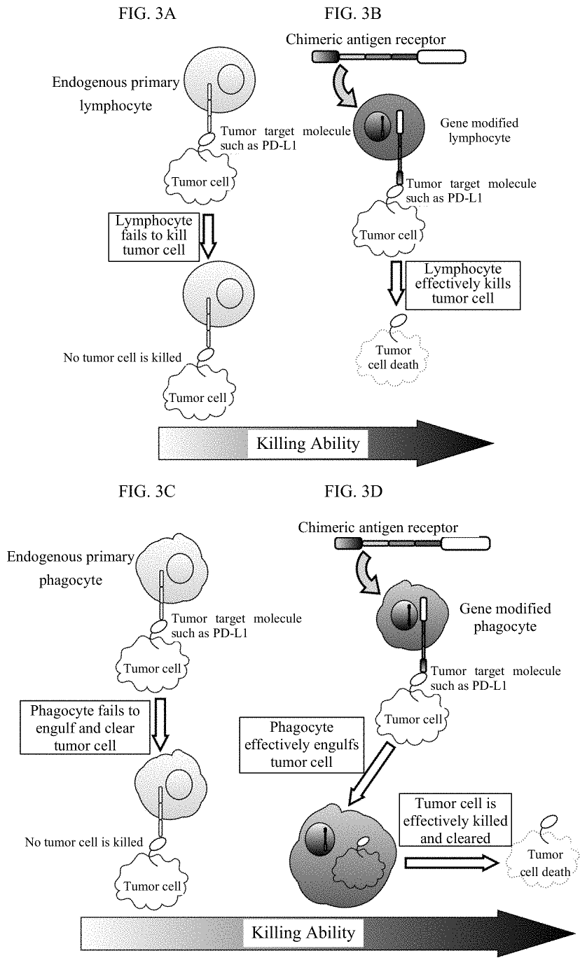

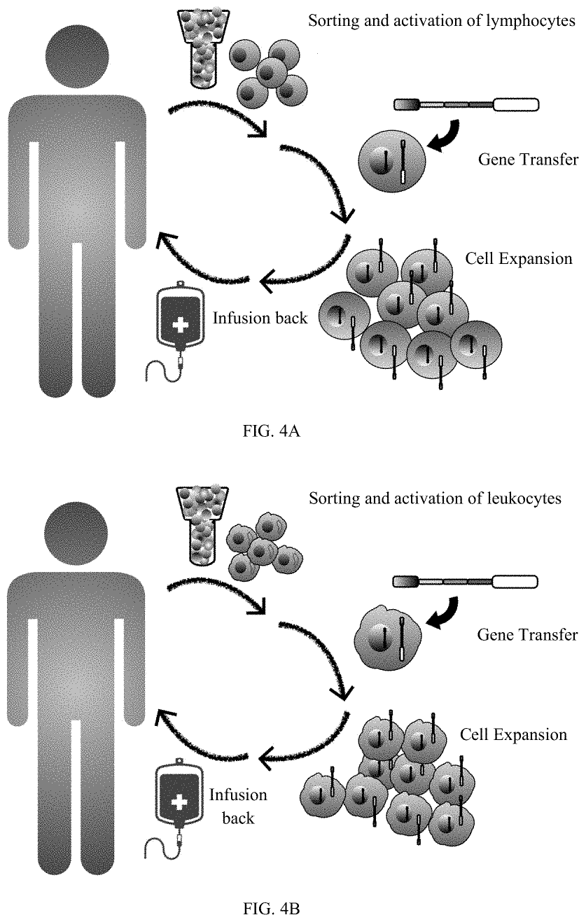

Through tumor cytotoxic killing experiments, the tumor killing detection of human immunogenic primary T lymphocytes or phagocytes against PD-L1 positive human cancer cells modified by the immune checkpoint PD-1 fusion-based chimeric antigen receptor is understanded, and the mechanisms thereof are shown in FIGS. 3A-3D respectively. FIG. 3A and FIG. 3C show that the ability of endogenous natural lymphocytes to kill corresponding cancer cells and natural phagocytes to phagocytose and clear corresponding cancer cells is inhibited by inhibitory immune checkpoint signaling pathways, when the immune checkpoint receptors (such as endogenous PD-1) on the surface of endogenous natural lymphocytes and natural phagocytes identify and bind to the targeting molecules (such as PD-L1) on the surface of cancer cells respectively. FIG. 3B and FIG. 3D show that the modified T lymphocytes and phagocytes can be effectively activated and effectively kill or phagocytose the corresponding cancer cells, when the human T lymphocytes and phagocytes modified by the immune checkpoint PD-1 fusion-based chimeric antigen receptor identify and bind to the targeting molecules PD-L1 on the surface of cancer cells respectively; wherein, the human cancer cells used in the cancer cell killing experiments in vitro are modified to express the reporter gene firefly luciferase, and the luciferase in cancer cells can accurately reflect the overall cell survival rate (Fu et al., PLoS ONE, 2010, 5: e11867; Ma et al, Oncotarget, 2016, 7: 29480-29491; Chen et al, Oncotarget, 2016, 7: 27764-27777.). that is, the surviving number of cancer cells are quantified by detecting the level of luciferase activity in cancer cells.

### (1) Experiment on the Killing Ability of Human Immunogenic Primary T Cells Modified by Immune Checkpoint PD-1 Fusion-Based Chimeric Antigen Receptor Against Cancer Cells

### Chimeric Antigen Receptor Expression in Human Primary T Cells Modified by Immune Checkpoint PD-1 Fusion-Based Chimeric Antigen Receptor:

Lentivirus packaging is used to prepare viral particles of different immune checkpoint PD-1 fusion-based chimeric antigen receptor artificial molecular machines; that is, the retrovirus expression vector (such as pSIN plasmid) and the packaging plasmid (such as psPAX2 and pMD2.G, or pCMV delta R8.2 and pCMV-VSV-G) carried with different immune checkpoint PD-1 fusion-based chimeric antigen receptor artificial molecular machines are used to transfect 293T cells, to obtain the virus supernatant which is then subjected to filtration, subpackaging and freezing to determine the virus titer. The isolation, activation and infection of human primary T cells are performed by the followings: isolating PBMCs (Peripheral Blood Mononuclear Cells) from the peripheral blood of healthy human by Ficoll density gradient centrifugation, subpackaging and freezing them in liquid nitrogen; rapidly recovering 3×10⁶˜10×10⁶PBMCs and using culture medium containing 2 ug/mL PHA to enrich and achieve the proliferation of activated T cells for 2˜3 days; coating a 6-well plate culture dish for non-tissue culture with 1%-2% Retronectin reagent at room temperature for 2 to 4 hours, then adding a certain amount of virus supernatant and activated T cells, and supplementing the culture medium containing human IL-2 (10˜50 U/ml), allowing the virus and T cells to bind to the bottom of the coated plate after incubation by centrifuging 1800 g for 60 minutes, and then infusing the same back to a 37° C. cell incubator to perform continuous culture for 5 to 6 days until they are used in subsequent operations. In the process of virus infection, fresh culture medium needs to be supplemented in time. Afterwards, PD-1 antibody staining is used to identify the human primary T cell population with high expression of chimeric antigen receptor fused with PD-1 on the surface thereof (see FIG. 12). Different immune checkpoint PD-1 fusion-based chimeric antigen receptors C #1, C #2, C #3, C #4 and C #5 have at least three-fold high level expression in human immunogenic primary T cell populations than that in the control groups (FIG. 12), and are used in co-culture experiments to detect cancer cell killing effect of human immunogenic primary T cells modified by different immune checkpoint PD-1 fusion-based chimeric antigen receptor against cancer cells. Please refer to FIG. 28 and related description according to the present application for the information of each component included in the immune checkpoint PD-1 fusion-based chimeric antigen receptor C #1 version, C #2 version, C #3 version, C #4 version and C #5 version.

### Tumor Killing Detection of Human Immunogenic Primary T Cells Modified by Immune Checkpoint PD-1 Fusion-Based Chimeric Antigen Receptor C #3 Version or C #5 Version Against PD-L1 Positive Human Colorectal Cancer Cells DLD1:

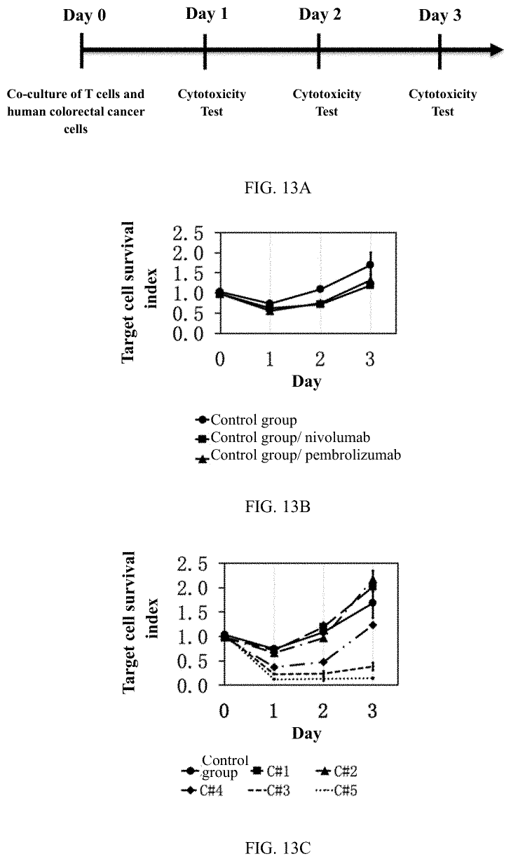

Human colorectal cancer cells DLD1 expressing the reporter gene firefly luciferase are pretreated with 500 U/mL interferon-γ for 24 hours to increase the expression of PD-L1 on the surface thereof. 1×10⁴ modified human immunogenic primary T cells and 1×10³ cancer cells are co-cultured in a 24-well plate at an E/T (effector cell/target cell) ratio of 10:1 for 24 to 72 hours, wherein the time when the co-culture starts is day 0. Then, the corresponding luciferase activity is measured by fluorescence spectrophotometer at three co-culture time points 24 hours, 48 hours and 72 hours after incubation to quantify the killing degree of human immunogenic primary T cells modified by the immune checkpoint PD-1 fusion-based chimeric antigen receptor C #3 version or C #5 version against cancer cells. Please refer to FIGS. 3A-3D and FIGS. 13A-13C. FIG. 13C shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human colorectal cancer cells, wherein, at 72 hours after incubation (the average value of C #3 group is 0.384, the average value of C #5 group is 0.144, the average value of control group is 1.687, the average value of C #1 group is 2.011, and the average of C #2 group is 2.174 and the average of C #4 group is 1.237), comparing with human immunogenic primary T cells in the control group, human immunogenic primary T cells modified by the immune checkpoint PD-1 fusion-based chimeric antigen receptor C #3 or C #5 show the greatest cancer cell clearance capacity, respectively, and the cell numbers of human cancer cells are 22% and 8% of the control group respectively. Quantitative analysis line diagrams prove that the immunogenic primary T cells modified by immune checkpoint PD-1 fusion-based chimeric antigen receptor C #3 or C #5 have significantly different and excellent ability to identify and kill cancer cells after statistical analysis under the condition that they are co-cultured with PD-L1-positive human cancer cells, while the immunogenic primary T cells in other experimental groups C #1, C #2, C #4 and the control group do not show the ability to effectively identify and kill cancer cells under the condition that they are co-cultured with PD-L1-positive human cancer cells.

### Tumor Killing Detection of PD-1 Immune Checkpoint Inhibitor Against PD-L1 Positive Human Colorectal Cancer Cells DLD1:

Human colorectal cancer cells DLD1 expressing the reporter gene firefly luciferase are first pretreated with interferon-γ for 24 hours to increase the expression of PD-L1 on the surface thereof, and then inoculated in appropriate culture dish on the day of the experiment; then human immunogenic primary T cells and an anti-PD-1 monoclonal antibody immune checkpoint inhibitor are added to the culture dish that has been inoculated with the human colorectal cancer cells, which is recorded as day 0, then incubated 24 hours, 48 hours and 72 hours respectively to detect luciferase activity of cancer cells in the cell culture system at these three co-culture time points, the number of human colorectal cancer cells is quantified and the cytotoxicity of the human immunogenic primary T cells against human colorectal cancers is calculated. Please refer to FIGS. 13A-13C. Quantitative analysis line diagrams in FIG. 13B show that, at post-incubation 72 hours (the average of the control/nivolumab group is 1.184, the average of the control/pembrolizumab group is 1.314, and the average of the control group is 1.687), nivolumab or pembrolizumab as PD-1 immune checkpoint inhibitor and human immunogenic primary T cells have limited cancer cell clearance capacity, and the numbers of human cancer cells are 70% and 78% of the control group, respectively, proving that the blocking of the PD-1/PD-L1 signaling pathway by PD-1 immune checkpoint inhibitors can improve the cytotoxic effect of immunogenic primary T cells against the PD-L1-positive colorectal cancer cells DLD1 to a certain extent, but such cytotoxic effect is significantly weaker than the cell therapy based on C #3 and C #5 in the present application.

### Tumor Killing Detection of Human Immunogenic Primary T Cells Modified by Immune Checkpoint PD-1 Fusion-Based Chimeric Antigen Receptor C #3 Version or C #5 Version Against Human Breast Cancer Cells MDA-MB-231:

The human breast cancer cells MDA-MB-231 used in the following tumor killing experiments are those not pretreated with interferon-γ and those pretreated with interferon-γ respectively. MDA-MB-231 cancer cells belong to the cancer cell type that can respond to interferon-γ stimulation and significantly upregulate the expression level of PD-L1 on the surface thereof (Soliman H et al., PloS one. 2014 Feb. 14; 9(2):e88557.), and thus the expression level of PD-L1 on the surface of cells pretreated with interferon-γ is significantly lower than that of the cells pretreated with interferon-γ. Here, tumor killing experiments are performed by comparing cancer cells which are not pretreated with interferon-γ with those pretreated with interferon-γ, so as to fully detect and characterize the dependence of the immunogenic primary T cells modified by chimeric antigen receptor on PD-L1 expression level in terms of the corresponding cancer cell killing ability.

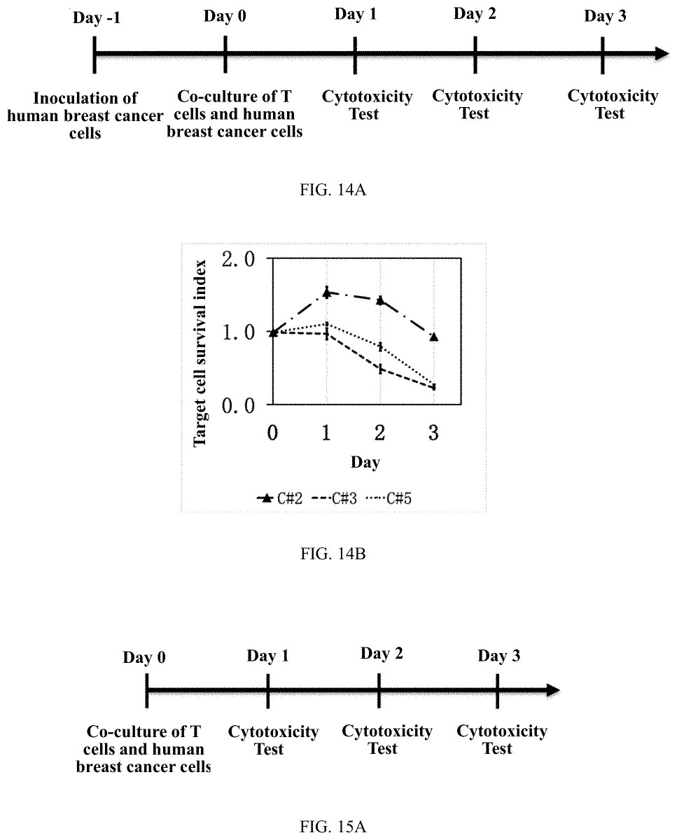

Human breast cancer cells MDA-MB-231 expressing the reporter gene firefly luciferase which are not pretreated with interferon-γ are used as tumor target cells to detect the killing ability of the immunogenic primary T cells modified by the immune checkpoint PD-1 fusion-based chimeric antigen receptor against the corresponding cancer cells. 1×10⁴ modified human immunogenic primary T cells and 1×10³ cancer cells are co-cultured in a 24-well plate at an E/T (effector cell/target cell) ratio of 10:1 for 24 to 72 hours, wherein the time when the co-culture starts is day 0. Then, the corresponding luciferase activity is measured by fluorescence spectrophotometer at three co-culture time points 24 hours, 48 hours and 72 hours after incubation to quantify the killing degree of human immunogenic primary T cells modified by the immune checkpoint PD-1 fusion-based chimeric antigen receptor C #3 version or C #5 version against cancer cells. Please refer to FIGS. 14A-14B. FIG. 14B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human cancer cells, wherein, at 72 hours after incubation (the average value of C #3 group is 0.233, the average value of C #5 group is 0.278, and the average of C #2 group is 0.928), comparing with human immunogenic primary T cells in the control group, human immunogenic primary T cells modified by the immune checkpoint PD-1 fusion-based chimeric antigen receptor C #3 or C #5 show the greatest cancer cell clearance capacity, respectively, and the cell numbers of human cancer cells are 25% and 30% of the control group respectively. Quantitative analysis line diagrams prove that, under the condition that interferon-γ pretreatment is not performed to enhance the PD-L1 expression on the surface of cancer cells, the immunogenic primary T cells modified by immune checkpoint PD-1 fusion-based chimeric antigen receptor C #3 or C #5 still have significantly different and excellent ability to identify and kill cancer cells after statistical analysis when they are co-cultured with PD-L1 positive human cancer cells, while the immunogenic primary T cells in other experimental group C #2 have significantly weak ability to identify and kill cancer cells when they are co-cultured with the same PD-L1 positive human cancer cells.

The human breast cancer cells MDA-MB-231 used in the following experiments are pretreated with interferon-γ for 24 hours, and thus the expression level of PD-L1 on the surface of cancer cells is higher than that on the surface of cells which are not pretreated with interferon-γ (Soliman H et al, PloS one. 2014 Feb. 14; 9(2):e88557.).

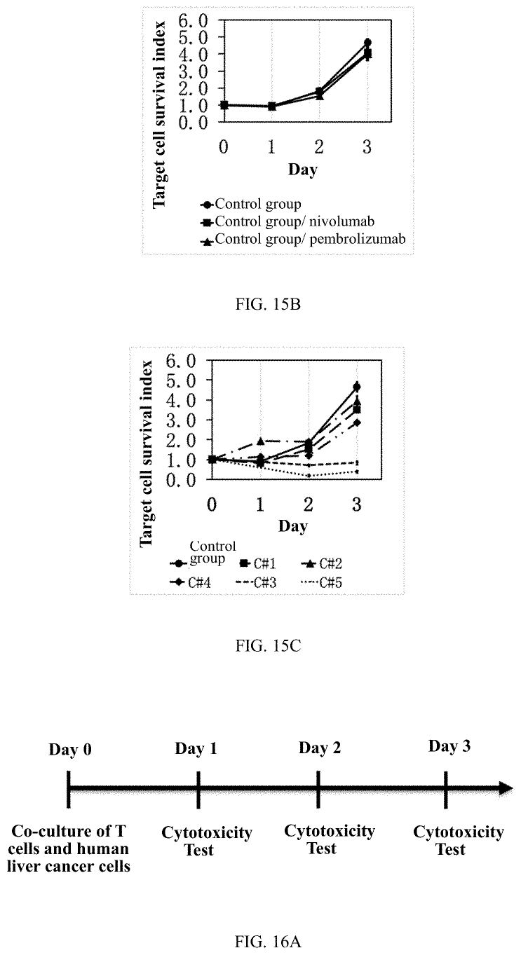

Human breast cancer cells MDA-MB-231 expressing the reporter gene firefly luciferase are first pretreated with 500 U/mL interferon-γ for 24 hours to increase the expression of PD-L1 on the surface thereof. 1×10⁴ modified human immunogenic primary T cells and 1×10³ cancer cells are co-cultured in a 24-well plate at an E/T (effector cell/target cell) ratio of 10:1 for 24 to 72 hours, wherein the time when the co-culture starts is day 0. Then, the corresponding luciferase activity is measured by fluorescence spectrophotometer at three co-culture time points 24 hours, 48 hours and 72 hours after incubation to quantify the killing degree of human immunogenic primary T cells modified by the immune checkpoint PD-1 fusion-based chimeric antigen receptor C #3 version or C #5 version against cancer cells. Please refer to FIGS. 15A-15C. FIG. 15C shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human colorectal cancer cells, wherein, at 72 hours after incubation (the average value of C #3 group is 0.843, the average value of C #5 group is 0.389, the average value of control group is 4.657, the average value of C #1 group is 3.487, and the average of C #2 group is 3.934 and the average of C #4 group is 2.855), comparing with human immunogenic primary T cells in the control group, human immunogenic primary T cells modified by the immune checkpoint PD-1 fusion-based chimeric antigen receptor C #3 or C #5 show the greatest cancer cell clearance capacity, respectively, and the cell numbers of human cancer cells are 18% and 8% of the control group respectively. Quantitative analysis line diagrams prove that the immunogenic primary T cells modified by immune checkpoint PD-1 fusion-based chimeric antigen receptor C #3 or C #5 have significantly different and excellent ability to identify and kill cancer cells after statistical analysis under the condition that they are co-cultured with PD-L1-positive human cancer cells, while the immunogenic primary T cells in other experimental groups C #1, C #2, C #4 and the control group do not show the ability to effectively identify and kill cancer cells under the condition that they are co-cultured with PD-L1-positive human cancer cells.

### Tumor Killing Detection of PD-1 Immune Checkpoint Inhibitor Against PD-L1 Positive Human Breast Cancer Cells MDA-MB-231:

Human breast cancer cells MDA-MB-231 expressing the reporter gene firefly luciferase are pretreated with interferon-γ for 24 hours to increase the expression of PD-L1 on the surface thereof, and are inoculated in appropriate culture dish on the day of the experiment, then human immunogenic primary T cells and an anti-PD-1 monoclonal antibody immune checkpoint inhibitor are added to the culture dish that has been inoculated with the human breast cancer cells, which is recorded as day 0, then incubated 24 hours, 48 hours and 72 hours respectively to detect luciferase activity of cancer cells in the cell culture system at these three co-culture time points, the number of human breast cancer cells is quantified and the cytotoxicity of the human immunogenic primary T cells against human breast cancer is calculated. Please refer to FIGS. 15A-15C. Quantitative analysis line diagrams in FIG. 15B show that, at post-incubation 72 hours (the average of the control/nivolumab group is 4.215, the average of the control/pembrolizumab group is 4.180, and the average of the control group is 5.010), nivolumab or pembrolizumab as PD-1 immune checkpoint inhibitor and human immunogenic primary T cells have limited cancer cell clearance capacity, and the numbers of human cancer cells are 87% and 86% of the control group, respectively, proving that the blocking of the PD-1/PD-L1 signaling pathway by PD-1 immune checkpoint inhibitors can improve the cytotoxic effect of immunogenic primary T cells against the PD-L1-positive breast cancer cells MDA-MB-231 to a certain extent, but such cytotoxic effect is significantly weaker than the cell therapy based on C #3 and C #5 in the present application.

### Tumor Killing Detection of Human Immunogenic Primary T Cells Modified by Immune Checkpoint PD-1 Fusion-Based Chimeric Antigen Receptor C #3 Version or C #5 Version Against PD-L1 Positive Human Liver Cancer Cancer Cells HA22T:

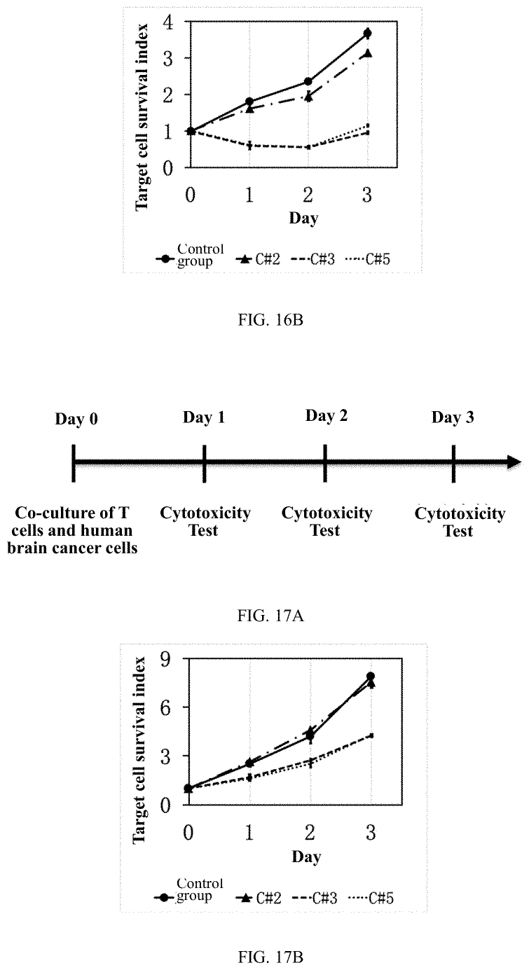

Human liver cancer cancer cells HA22T expressing the reporter gene firefly luciferase are pretreated with interferon-γ for 24 hours to increase the expression of PD-L1 on the surface thereof. 1×10⁴ modified human immunogenic primary T cells and 1×10³ cancer cells are co-cultured in a 24-well plate at an E/T (effector cell/target cell) ratio of 10:1 for 24 to 72 hours, wherein the time when the co-culture starts is day 0. Then, the corresponding luciferase activity is measured by fluorescence spectrophotometer at three co-culture time points 24 hours, 48 hours and 72 hours after incubation to quantify the killing degree of human immunogenic primary T cells modified by the immune checkpoint PD-1 fusion-based chimeric antigen receptor C #3 version or C #5 version against cancer cells. Please refer to FIGS. 16A-16B. FIG. 16B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human cancer cells, wherein, at 72 hours after incubation (the average value of C #3 group is 0.953, the average value of C #5 group is 1.153, the average value of control group is 3.665, the average of C #2 group is 3.143), comparing with human immunogenic primary T cells in the control group, human immunogenic primary T cells modified by the immune checkpoint PD-1 fusion-based chimeric antigen receptor C #3 or C #5 show the greatest cancer cell clearance capacity, respectively, and the cell numbers of human cancer cells are 26% and 31% of the control group respectively. Quantitative analysis line diagrams prove that the immunogenic primary T cells modified by immune checkpoint PD-1 fusion-based chimeric antigen receptor C #3 or C #5 still have significantly different and excellent ability to identify and kill cancer cells after statistical analysis when they are co-cultured with PD-L1 positive human cancer cells, while the immunogenic primary T cells in other experimental group C #2 and the control group do not show the ability to effectively identify and kill cancer cells when they are co-cultured with the PD-L1 positive human cancer cells.

### Tumor Killing Detection of Human Immunogenic Primary T Cells Modified by Immune Checkpoint PD-1 Fusion-Based Chimeric Antigen Receptor C #3 Version or C #5 Version Against PD-L1 Positive Human Brain Cancer Cells U87-MG:

Human brain cancer cells U87-MG expressing the reporter gene firefly luciferase are first pretreated with interferon-γ for 24 hours to increase the expression of PD-L1 on the surface thereof. 1×10⁴ modified human immunogenic primary T cells and 1×10³ cancer cells are co-cultured in a 24-well plate at an E/T (effector cell/target cell) ratio of 10:1 for 24 to 72 hours, wherein the time when the co-culture starts is day 0. Then, the corresponding luciferase activity is measured by fluorescence spectrophotometer at three co-culture time points 24 hours, 48 hours and 72 hours after incubation to quantify the killing degree of human immunogenic primary T cells modified by the immune checkpoint PD-1 fusion-based chimeric antigen receptor C #3 version or C #5 version against cancer cells. Please refer to FIGS. 17A-17B. FIG. 17B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human cancer cells, wherein, at 72 hours after incubation (the average value of C #3 group is 4.258, the average value of C #5 group is 4.300, the average value of control group is 7.885, the average of C #2 group is 7.558), comparing with human immunogenic primary T cells in the control group, human immunogenic primary T cells modified by the immune checkpoint PD-1 fusion-based chimeric antigen receptor C #3 or C #5 show the greatest cancer cell clearance capacity, respectively, and the cell numbers of human cancer cells are 54% and 55% of the control group respectively. Quantitative analysis line diagrams prove that the immunogenic primary T cells modified by immune checkpoint PD-1 fusion-based chimeric antigen receptor C #3 or C #5 still have significantly different and excellent ability to identify and kill cancer cells after statistical analysis when they are co-cultured with PD-L1 positive human cancer cells, while the immunogenic primary T cells in other experimental group C #2 and the control group do not show the ability to effectively identify and kill cancer cells when they are co-cultured with the PD-L1 positive human cancer cells.

### Tumor Killing Detection of Human Immunogenic Primary T Cells Modified by Immune Checkpoint PD-1 Fusion-Based Chimeric Antigen Receptor C #3 Version or C #5 Version Against PD-L1 Positive Human Skin Cancer Cells A2058:

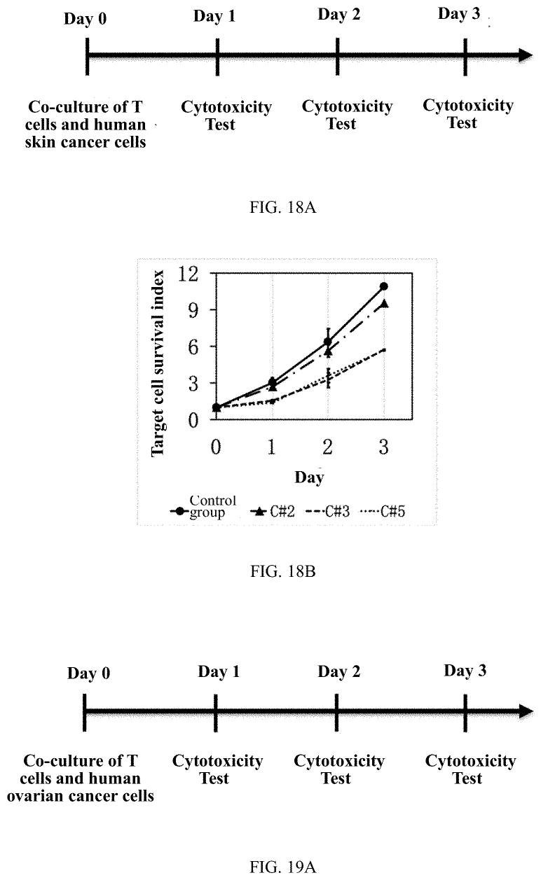

Human skin cancer cells A2058 expressing the reporter gene firefly luciferase are first pretreated with interferon-γ for 24 hours to increase the expression of PD-L1 on the surface thereof. 1×10⁴ modified human immunogenic primary T cells and 1×10³ cancer cells are co-cultured in a 24-well plate at an E/T (effector cell/target cell) ratio of 10:1 for 24 to 72 hours, wherein the time when the co-culture starts is day 0. Then, the corresponding luciferase activity is measured by fluorescence spectrophotometer at three co-culture time points 24 hours, 48 hours and 72 hours after incubation to quantify the killing degree of human immunogenic primary T cells modified by the immune checkpoint PD-1 fusion-based chimeric antigen receptor C #3 version or C #5 version against cancer cells. Please refer to FIGS. 18A-18B. FIG. 18B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human cancer cells, wherein, at 72 hours after incubation (the average value of C #3 group is 5.773, the average value of C #5 group is 5.670, the average value of control group is 10.920, the average of C #2 group is 9.513), comparing with human immunogenic primary T cells in the control group, human immunogenic primary T cells modified by the immune checkpoint PD-1 fusion-based chimeric antigen receptor C #3 or C #5 show the greatest cancer cell clearance capacity, respectively, and the cell numbers of human cancer cells are 53% and 52% of the control group respectively. Quantitative analysis line diagrams prove that the immunogenic primary T cells modified by immune checkpoint PD-1 fusion-based chimeric antigen receptor C #3 or C #5 still have significantly different and excellent ability to identify and kill cancer cells after statistical analysis when they are co-cultured with PD-L1 positive human cancer cells, while the immunogenic primary T cells in other experimental group C #2 and the control group do not show the ability to effectively identify and kill cancer cells when they are co-cultured with the PD-L1 positive human cancer cells.

### Tumor Killing Detection of Human Immunogenic Primary T Cells Modified by Immune Checkpoint PD-1 Fusion-Based Chimeric Antigen Receptor C #3 Version or C #5 Version Against PD-L1 Positive Human Ovarian Cancer Cells ES-2:

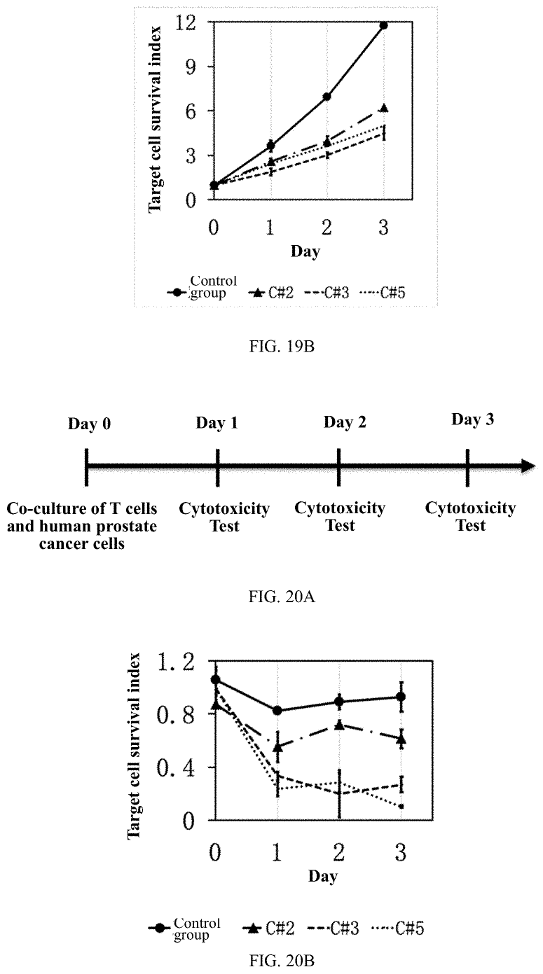

Human ovarian cancer cells ES-2 expressing the reporter gene firefly luciferase are pretreated with interferon-γ for 24 hours to increase the expression of PD-L1 on the surface thereof. 1×10⁴ modified human immunogenic primary T cells and 1×10³ cancer cells are co-cultured in a 24-well plate at an E/T (effector cell/target cell) ratio of 10:1 for 24 to 72 hours, wherein the time when the co-culture starts is day 0. Then, the corresponding luciferase activity is measured by fluorescence spectrophotometer at three co-culture time points 24 hours, 48 hours and 72 hours after incubation to quantify the killing degree of human immunogenic primary T cells modified by the immune checkpoint PD-1 fusion-based chimeric antigen receptor C #3 version or C #5 version against cancer cells. Please refer to FIGS. 19A-19B. FIG. 19B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human cancer cells, wherein, at 72 hours after incubation (the average value of C #3 group is 4.480, the average value of C #5 group is 5.008, the average value of control group is 11.720, the average of C #2 group is 6.210), comparing with human immunogenic primary T cells in the control group, human immunogenic primary T cells modified by the immune checkpoint PD-1 fusion-based chimeric antigen receptor C #3 or C #5 show the greatest cancer cell clearance capacity, respectively, and the cell numbers of human cancer cells are 40% and 46% of the control group respectively. Quantitative analysis line diagrams prove that the immunogenic primary T cells modified by immune checkpoint PD-1 fusion-based chimeric antigen receptor C #3 or C #5 still have significantly different and excellent ability to identify and kill cancer cells after statistical analysis when they are co-cultured with PD-L1 positive human cancer cells, while the immunogenic primary T cells in other experimental group C #2 and the control group do not show the ability to effectively identify and kill cancer cells when they are co-cultured with the PD-L1 positive human cancer cells.

### Tumor Killing Detection of Human Immunogenic Primary T Cells Modified by Immune Checkpoint PD-1 Fusion-Based Chimeric Antigen Receptor C #3 Version or C #5 Version Against PD-L1 Positive Human Prostate Cancer Cells PC-3:

Human prostate cancer cells PC-3 expressing the reporter gene firefly luciferase are first pretreated with interferon-γ for 24 hours to increase the expression of PD-L1 on the surface thereof. 1×10⁴ modified human immunogenic primary T cells and 1×10³ cancer cells are co-cultured in a 24-well plate at an E/T (effector cell/target cell) ratio of 10:1 for 24 to 72 hours, wherein the time when the co-culture starts is day 0. Then, the corresponding luciferase activity is measured by fluorescence spectrophotometer at three co-culture time points 24 hours, 48 hours and 72 hours after incubation to quantify the killing degree of human immunogenic primary T cells modified by the immune checkpoint PD-1 fusion-based chimeric antigen receptor C #3 version or C #5 version against cancer cells. Please refer to FIGS. 20A-20B. FIG. 20B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human cancer cells, wherein, at 72 hours after incubation (the average value of C #3 group is 0.270, the average value of C #5 group is 0.105, the average value of control group is 0.925, the average of C #2 group is 0.615), comparing with human immunogenic primary T cells in the control group, human immunogenic primary T cells modified by the immune checkpoint PD-1 fusion-based chimeric antigen receptor C #3 or C #5 show the greatest cancer cell clearance capacity, respectively, and the cell numbers of human cancer cells are 29% and 11% of the control group respectively. Quantitative analysis line diagrams prove that the immunogenic primary T cells modified by immune checkpoint PD-1 fusion-based chimeric antigen receptor C #3 or C #5 still have significantly different and excellent ability to identify and kill cancer cells after statistical analysis when they are co-cultured with PD-L1 positive human cancer cells, while the immunogenic primary T cells in other experimental group C #2 and the control group do not show the ability to effectively identify and kill cancer cells when they are co-cultured with the PD-L1 positive human cancer cells.

### Tumor Killing Detection of Human Immunogenic Primary T Cells Modified by Immune Checkpoint PD-1 Fusion-Based Chimeric Antigen Receptor C #3 Version or C #5 Version Against PD-L1 Positive Human Pancreatic Cancer Cells AsPC1:

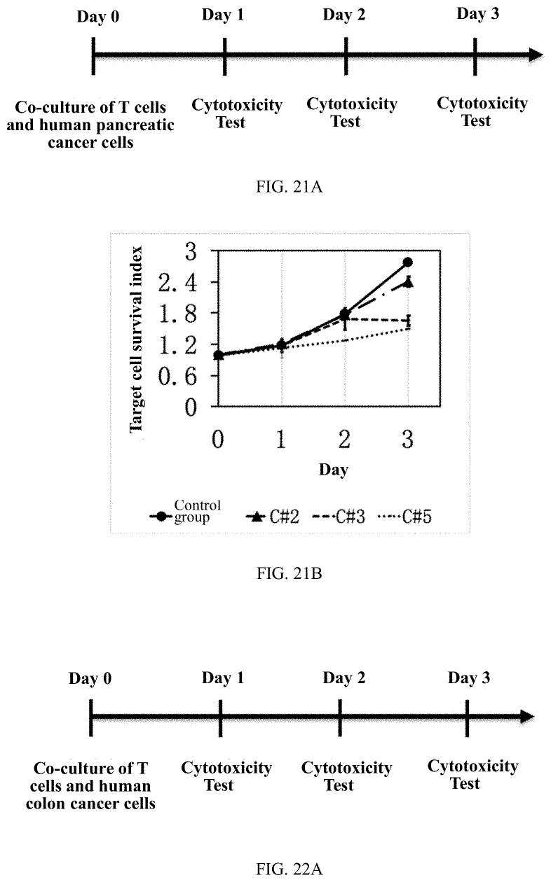

Human pancreatic cancer cells AsPC1 expressing the reporter gene firefly luciferase are first pretreated with interferon-γ for 24 hours to increase the expression of PD-L1 on the surface thereof. 1×10⁴ modified human immunogenic primary T cells and 1×10³ cancer cells are co-cultured in a 24-well plate at an E/T (effector cell/target cell) ratio of 10:1 for 24 to 72 hours, wherein the time when the co-culture starts is day 0. Then, the corresponding luciferase activity is measured by fluorescence spectrophotometer at three co-culture time points 24 hours, 48 hours and 72 hours after incubation to quantify the killing degree of human immunogenic primary T cells modified by the immune checkpoint PD-1 fusion-based chimeric antigen receptor C #3 version or C #5 version against cancer cells. Please refer to FIGS. 21A-21B. FIG. 21B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human cancer cells, wherein, at 72 hours after incubation (the average value of C #3 group is 1.653, the average value of C #5 group is 1.495, the average value of control group is 2.765, the average of C #2 group is 2.398), comparing with human immunogenic primary T cells in the control group, human immunogenic primary T cells modified by the immune checkpoint PD-1 fusion-based chimeric antigen receptor C #3 or C #5 show the greatest cancer cell clearance capacity, respectively, and the cell numbers of human cancer cells are 60% and 54% of the control group respectively. Quantitative analysis line diagrams prove that the immunogenic primary T cells modified by immune checkpoint PD-1 fusion-based chimeric antigen receptor C #3 or C #5 still have significantly different and excellent ability to identify and kill cancer cells after statistical analysis when they are co-cultured with PD-L1 positive human cancer cells, while the immunogenic primary T cells in other experimental group C #2 and the control group do not show the ability to effectively identify and kill cancer cells when they are co-cultured with the PD-L1 positive human cancer cells.

### Tumor Killing Detection of Human Immunogenic Primary T Cells Modified by Immune Checkpoint PD-1 Fusion-Based Chimeric Antigen Receptor C #3 Version or C #5 Version Against PD-L1 Positive Human Colon Cancer Cells COLO205:

Human colon cancer cells COLO205 expressing the reporter gene firefly luciferase are pretreated with interferon-γ for 24 hours to increase the expression of PD-L1 on the surface thereof. 1×10⁴ modified human immunogenic primary T cells and 1×10³ cancer cells are co-cultured in a 24-well plate at an E/T (effector cell/target cell) ratio of 10:1 for 24 to 72 hours, wherein the time when the co-culture starts is day 0. Then, the corresponding luciferase activity is measured by fluorescence spectrophotometer at three co-culture time points 24 hours, 48 hours and 72 hours after incubation to quantify the killing degree of human immunogenic primary T cells modified by the immune checkpoint PD-1 fusion-based chimeric antigen receptor C #3 version or C #5 version against cancer cells. Please refer to FIGS. 22A-22B. FIG. 22B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human cancer cells, wherein, at 72 hours after incubation (the average value of C #3 group is 0.663, the average value of C #5 group is 0.840, the average value of control group is 1.288, the average of C #2 group is 1.648), comparing with human immunogenic primary T cells in the control group, human immunogenic primary T cells modified by the immune checkpoint PD-1 fusion-based chimeric antigen receptor C #3 or C #5 show the greatest cancer cell clearance capacity, respectively, and the cell numbers of human cancer cells are 51% and 65% of the control group respectively. Quantitative analysis line diagrams prove that the immunogenic primary T cells modified by immune checkpoint PD-1 fusion-based chimeric antigen receptor C #3 or C #5 still have significantly different and excellent ability to identify and kill cancer cells after statistical analysis when they are co-cultured with PD-L1 positive human cancer cells, while the immunogenic primary T cells in other experimental group C #2 and the control group do not show the ability to effectively identify and kill cancer cells when they are co-cultured with the PD-L1 positive human cancer cells.

### Tumor Killing Detection of Human Immunogenic Primary T Cells Modified by Immune Checkpoint PD-1 Fusion-Based Chimeric Antigen Receptor C #3 Version or C #5 Version Against PD-L1 Positive Human Kidney Cancer Cells 786-O:

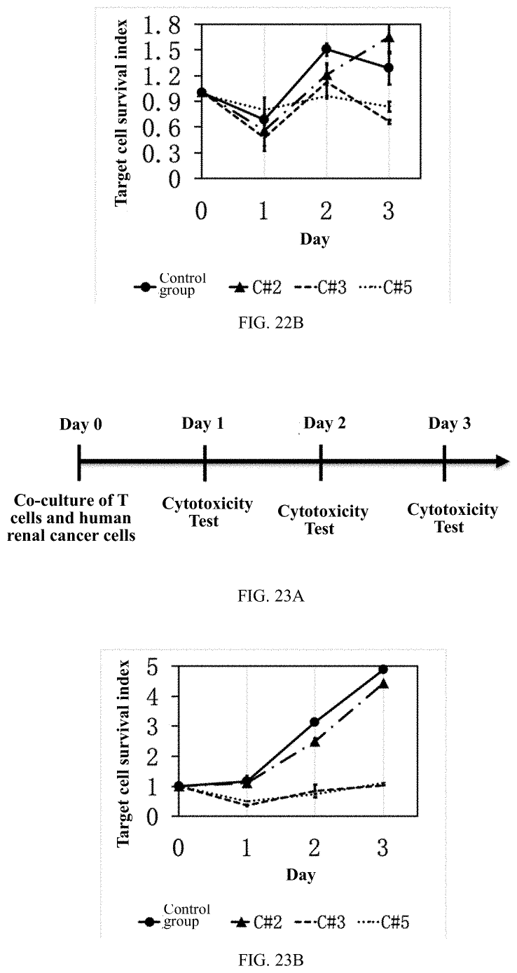

Human renal carcinoma cancer cells 786-O expressing the reporter gene firefly luciferase are pretreated with interferon-γ for 24 hours to increase the expression of PD-L1 on the surface thereof. 1×10⁴ modified human immunogenic primary T cells and 1×10³ cancer cells are co-cultured in a 24-well plate at an E/T (effector cell/target cell) ratio of 10:1 for 24 to 72 hours, wherein the time when the co-culture starts is day 0. Then, the corresponding luciferase activity is measured by fluorescence spectrophotometer at three co-culture time points 24 hours, 48 hours and 72 hours after incubation to quantify the killing degree of human immunogenic primary T cells modified by the immune checkpoint PD-1 fusion-based chimeric antigen receptor C #3 version or C #5 version against cancer cells. Please refer to FIGS. 23A-23B. FIG. 23B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human cancer cells, wherein, at 72 hours after incubation (the average value of C #3 group is 1.035, the average value of C #5 group is 1.095, the average value of control group is 4.878, the average of C #2 group is 4.418), comparing with human immunogenic primary T cells in the control group, human immunogenic primary T cells modified by the immune checkpoint PD-1 fusion-based chimeric antigen receptor C #3 or C #5 show the greatest cancer cell clearance capacity, respectively, and the cell numbers of human cancer cells are 21% and 22% of the control group respectively. Quantitative analysis line diagrams prove that the immunogenic primary T cells modified by immune checkpoint PD-1 fusion-based chimeric antigen receptor C #3 or C #5 still have significantly different and excellent ability to identify and kill cancer cells after statistical analysis when they are co-cultured with PD-L1 positive human cancer cells, while the immunogenic primary T cells in other experimental group C #2 and the control group do not show the ability to effectively identify and kill cancer cells when they are co-cultured with the PD-L1 positive human cancer cells.

### Tumor Killing Detection of Human Immunogenic Primary T Cells Modified by Immune Checkpoint PD-1 Fusion-Based Chimeric Antigen Receptor C #3 Version or C #5 Version Against PD-L1 Positive Human Lung Cancer Cells H441:

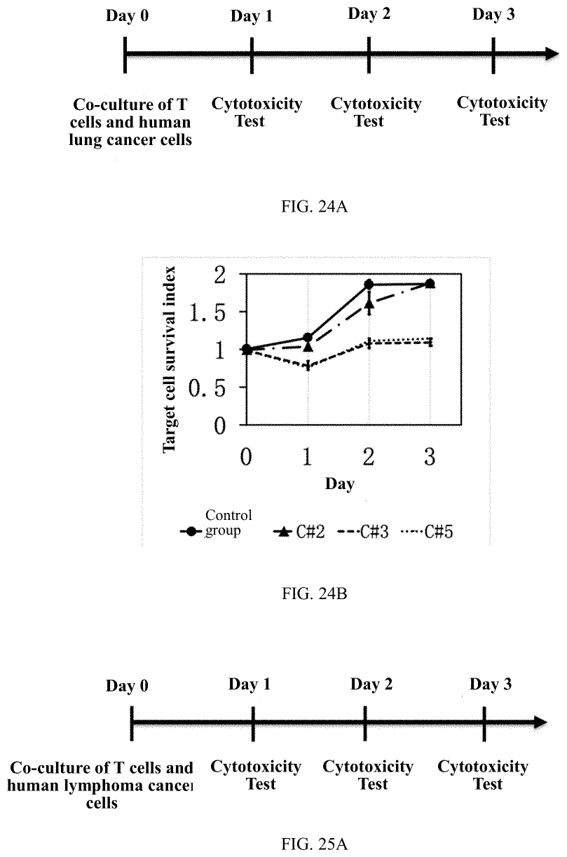

Human lung cancer cells H441 expressing the reporter gene firefly luciferase are first pretreated with interferon-γ for 24 hours to increase the expression of PD-L1 on the surface thereof. 1×10⁴ modified human immunogenic primary T cells and 1×10³ cancer cells are co-cultured in a 24-well plate at an E/T (effector cell/target cell) ratio of 10:1 for 24 to 72 hours, wherein the time when the co-culture starts is day 0. Then, the corresponding luciferase activity is measured by fluorescence spectrophotometer at three co-culture time points 24 hours, 48 hours and 72 hours after incubation to quantify the killing degree of human immunogenic primary T cells modified by the immune checkpoint PD-1 fusion-based chimeric antigen receptor C #3 version or C #5 version against cancer cells. Please refer to FIGS. 24A-24B. FIG. 24B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human cancer cells, wherein, at 72 hours after incubation (the average value of C #3 group is 1.095, the average value of C #5 group is 1.143, the average value of control group is 1.868, the average of C #2 group is 1.878), comparing with human immunogenic primary T cells in the control group, human immunogenic primary T cells modified by the immune checkpoint PD-1 fusion-based chimeric antigen receptor C #3 or C #5 show the greatest cancer cell clearance capacity, respectively, and the cell numbers of human cancer cells are 59% and 61% of the control group respectively. Quantitative analysis line diagrams prove that the immunogenic primary T cells modified by immune checkpoint PD-1 fusion-based chimeric antigen receptor C #3 or C #5 still have significantly different and excellent ability to identify and kill cancer cells after statistical analysis when they are co-cultured with PD-L1 positive human cancer cells, while the immunogenic primary T cells in other experimental group C #2 and the control group do not show the ability to effectively identify and kill cancer cells when they are co-cultured with the PD-L1 positive human cancer cells.

### Tumor Killing Detection of Human Immunogenic Primary T Cells Modified by Immune Checkpoint PD-1 Fusion-Based Chimeric Antigen Receptor C #3 Version or C #5 Version Against PD-L1 Positive Human Lymphoma Cancer Cells U937:

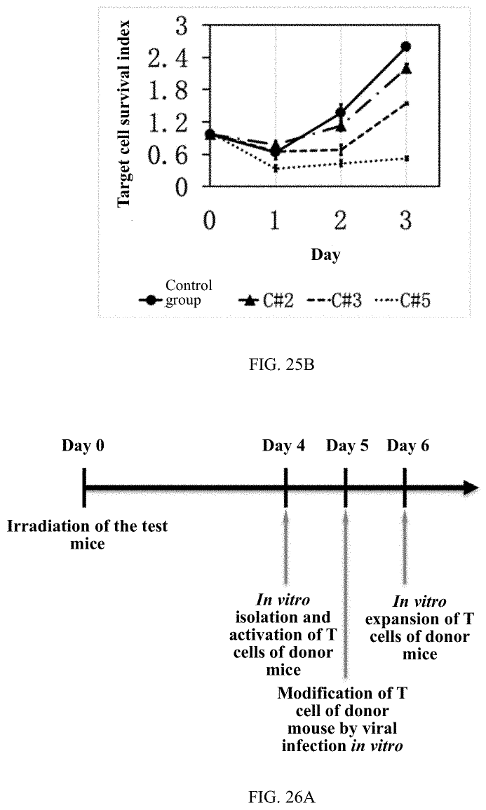

Human lymphoma cancer cells U937 expressing the reporter gene firefly luciferase are first pretreated with interferon-γ for 24 hours to increase the expression of PD-L1 on the surface thereof. 1×10⁴ modified human immunogenic primary T cells and 1×10³ cancer cells are co-cultured in a 24-well plate at an E/T (effector cell/target cell) ratio of 10:1 for 24 to 72 hours, wherein the time when the co-culture starts is day 0. Then, the corresponding luciferase activity is measured by fluorescence spectrophotometer at three co-culture time points 24 hours, 48 hours and 72 hours after incubation to quantify the killing degree of human immunogenic primary T cells modified by the immune checkpoint PD-1 fusion-based chimeric antigen receptor C #3 version or C #5 version against cancer cells. Please refer to FIGS. 25A-25B. FIG. 25B shows quantitative analysis result of the cytotoxic effect of the in vitro co-culture of human immunogenic primary T cell modified by different immune checkpoint PD-1 fusion-based chimeric antigen receptor artificial molecular machines and PD-L1 positive human cancer cells, wherein, at 72 hours after incubation (the average value of C #3 group is 1.548, the average value of C #5 group is 0.518, the average value of control group is 2.595, the average of C #2 group is 2.190), comparing with human immunogenic primary T cells in the control group, human immunogenic primary T cells modified by the immune checkpoint PD-1 fusion-based chimeric antigen receptor C #3 or C #5 show the greatest cancer cell clearance capacity, respectively, and the cell numbers of human cancer cells are 59% and 20% of the control group respectively. Quantitative analysis line diagrams prove that the immunogenic primary T cells modified by immune checkpoint PD-1 fusion-based chimeric antigen receptor C #3 or C #5 still have significantly different and excellent ability to identify and kill cancer cells after statistical analysis when they are co-cultured with PD-L1 positive human cancer cells, while the immunogenic primary T cells in other experimental group C #2 and the control group do not show the ability to effectively identify and kill cancer cells when they are co-cultured with the PD-L1 positive human cancer cells.

In summary, through the verification of cytotoxicity killing experiments of various tumors, the immunogenic primary T cells modified by the immune checkpoint PD-1 fusion-based chimeric antigen receptor exhibit excellent killing ability against cancer cells, especially against PD-L1 positive human cancer cells, as shown in FIGS. 3A-3D; wherein, the functionality of the C #3 version and C #5 version, i.e. the Truncated PD-1-Sub1-LL1-ZAP70 version and the Truncated PD-1-Sub5-LL1-SYK version, are particularly prominent. In addition, the C #4 version is an intracellular activation signaling domain mutant (ZAP70 ΔKD) of the C #3 version, that is, the intracellular activation signaling domain in the C #4 version is in a malfunctioning state. In the verification of cytotoxic killing experiments of various tumors, the immune T cells modified by C #4 version cannot effectively kill cancer cells, proving that the necessity and importance of the intracellular activation signaling domain of the chimeric antigen receptor to fully perform function for the chimeric antigen receptor. Finally, FIGS. 14A-14B and FIGS. 15A-15C prove that the C #3 and C #5 versions of the cell therapy according to the present application have excellent tumor killing ability against both PD-L1-positive cancer cells and the cancer cells that up-regulate PD-L1 expression level in response to interferon-γ. Especially, the cancer cells that up-regulate the expression level of PD-L1 in response to interferon-γ simulates the immunosuppressive tumor microenvironment in real patients to a certain extent, which provides more forward-looking supporting data in a better application of the cell therapy according to the present application in future clinical treatment.

(2) The Experiments of Phagocytosis and Killing Ability by Phagocytes (Including Monocytes and Macrophages) Modified with the Immune Checkpoint PD-1 Fusion-Based Chimeric Antigen Receptor Against the Cancer Cells

### Expression of Chimeric Antigen Receptor in Monocytes Modified by Immune Checkpoint PD-1 Fusion-Based Chimeric Antigen Receptor:

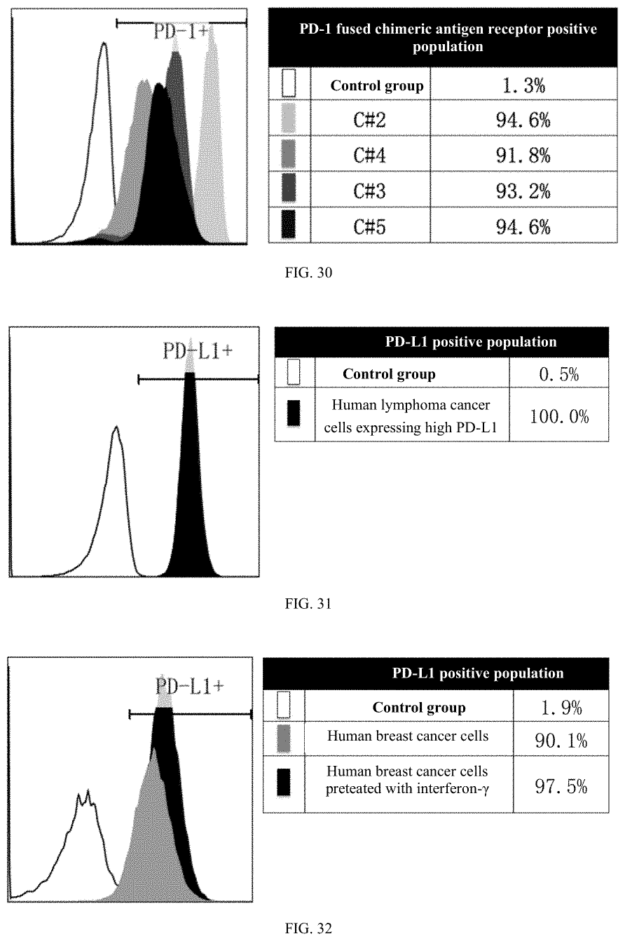

Lentivirus packaging is used to prepare viral particles of different immune checkpoint PD-1 fusion-based chimeric antigen receptor artificial molecular machines; that is, the retrovirus expression vector (such as pSIN plasmid) and the packaging plasmid (such as psPAX2 and pMD2.G, or pCMV delta R8.2 and pCMV-VSV-G) carried with different immune checkpoint PD-1 fusion-based chimeric antigen receptor artificial molecular machines are used to transfect 293T cells, to obtain the virus supernatant which is then subjected to filtration, subpackaging and freezing to determine the virus titer. A certain amount of virus supernatant is added to the culture dish of human monocytes THP1 for 24 hours, and the virus solution is discarded the next day. On day 2 to day 3 after virus infection of monocytes, PD-1 antibody staining is used to screen out the monocyte THP1 population with high expression of PD-1-fused chimeric antigen receptor on the surface thereof (see FIG. 30). Comparing with the control group, different immune checkpoint PD-1 fusion-based chimeric antigen receptors C #2, C #4, C #3 and C #5 are expressed more than 90% in monocytes THP1, and used in co-culture experiments to detect the cancer cell killing effect of the monocytes modified by different immune checkpoint PD-1 fusion-based chimeric antigen receptors respectively. Please refer to FIG. 28 and related description according to the present application for the information of each component included in the immune checkpoint PD-1 fusion-based chimeric antigen receptor C #2 version, C #4 version, C #3 version and C #5 version.

### Differentiation of Monocytes Modified by Immune Checkpoint PD-1 Fusion-Based Chimeric Antigen Receptor and the Expression of the Chimeric Antigen Receptor in Differentiated Macrophages:

Monocytes THP1 modified by different immune checkpoint PD-1 fusion-based chimeric antigen receptors respectively are induced by PMA (Phorbol 12-Myristate 13-Acetate, or phorbol ester) for at least 24 hours to be differentiated into macrophages for use in subsequent operations. In co-culture experiments, the tumor killing effect of the differentiated macrophages modified by different immune checkpoint PD-1 fusion-based chimeric antigen receptors C #2, C #3, C #4 and C #5 respectively is detected.

### Detection of the Expression Level of Immune Checkpoint Inhibitory Signaling Pathway Molecule PD-L1 on Different Cancer Cells:

PD-L1 antibody is used to stain and detect the expression of PD-L1 in human lymphoma cancer cell NALM6 modified strain, human breast cancer cells MBA-MB-231, and human colorectal cancer cell DLD1 modified strain. FIG. 31 shows that the expression ratio of PD-L1 on the surface of lymphoma cancer cell NALM6 modified strain is as high as 100% relative to the negative control group (Isotype Control), wherein, the lymphoma cancer cell NALM6 modified strain is used in cancer cell killing experiment. FIG. 32 shows the expression of PD-L1 in human breast cancer cells MBA-MB-231 and human breast cancer cells MDA-MB-231 pretreated by interferon-γ respectively, wherein, relative to the negative control group (Isotype Control), the expression ratio of PD-L1 in human breast cancer cells MBA-MB-231 is as high as 90.1%, while after pretreatment with interferon-γ of the human breast cancer cells MBA-MB-231, the expression ratio of PD-L1 increases to 97.5% which is significantly increased, further revealing that interferon-γ can promote the expression of PD-L1 on cancer cells, and the cancer cells are pretreated with interferon-γ in vitro experiments to simulate the tumor microenvironment in the body, and such pretreated human breast cancer cells MBA-MB-231 are used in cancer cell killing experiments. FIG. 33 shows the expression of PD-L1 in human colorectal cancer cell DLD1 modified strain relative to the negative control group (Isotype Control), and shows that the expression ratio of PD-L1 in colorectal cancer cells is as high as 98.7%, and the colorectal cancer cells DLD1 are used in cancer cell killing experiments.

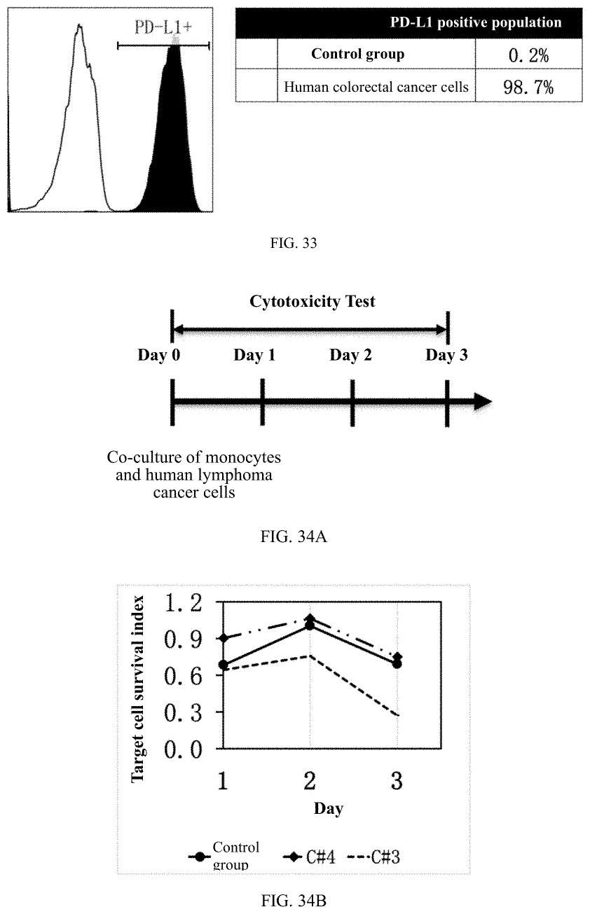

Tumor killing detection of monocytes modified by immune checkpoint PD-1 fusion-based chimeric antigen receptor C #3 version against PD-L1 positive human lymphoma cancer cells: 1×10⁴ modified human monocytes and 1×10³ cancer cells (human lymphoma cancer cell NALM6 modified strain with high PD-L1 expression) are co-cultured in a 24-well plate at an E/T (effector cell/target cell) ratio of 10:1 for 24 to 72 hours, wherein the time when the co-culture starts is day 0; wherein, all the different human cancer cells used are subjected to modification to express the reporter gene firefly luciferase. Then, the corresponding luciferase activity is measured by the fluorescence spectrophotometer at different co-culture time points, to quantify the killing degree of the differentiated monocytes against cancer cells. Please refer to FIGS. 34A-34B. FIG. 34B shows quantitative analysis result of the cytotoxic effect of the in vitro co-culture of human monocytes modified by different immune checkpoint PD-1 fusion-based chimeric antigen receptor artificial molecular machines respectively and PD-L1 positive human cancer cells, and quantitative analysis line diagrams prove that the monocytes modified by immune checkpoint PD-1 fusion-based chimeric antigen receptor C #3 version have excellent ability to identify and kill cancer cells when they are co-cultured with PD-L1 positive human cancer cells, especially on day 3 (the average of C #3 group is 0.274, and the average of the control group is 0.691), while the monocytes in other experimental group C #4 and the control group do not show the ability to effectively identify and kill cancer cells when they are co-cultured with the PD-L1 positive human cancer cells; wherein, the human monocyte in the control group is the one that has not been modified by the chimeric antigen receptor artificial molecular machine, and the target cell survival index represents relative cell number of human cancer cells expressing the reporter gene firefly luciferase in the cell culture system.

### Tumor Killing Detection of Macrophages Modified by Immune Checkpoint PD-1 Fusion-Based Chimeric Antigen Receptor C #5 Version Against PD-L1 Positive Human Breast Cancer Cells in the Presence of Antibody-Dependent Cell-Mediated Phagocytosis:

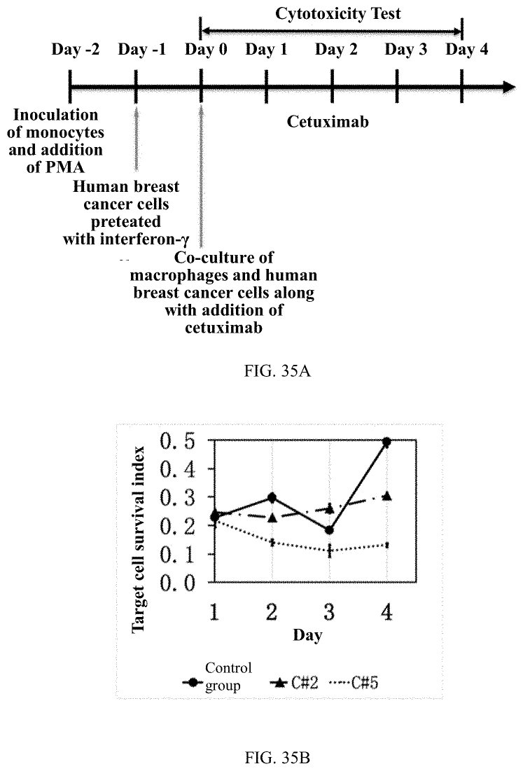

1×10⁴ modified human monocytes THP1 are first inoculated in a 24-well plate and then the phorbol ester PMA is added therein to differentiate the modified human monocytes THP1 into be macrophages; after 2 days, 1×10³ cancer cells (i.e. the modified human breast cancer cells MDA-MB-231 which are subjected to pretreatment with 500 U/mL interferon-γ for 24 hours) at an E/T (effector cell/target cell) ratio of 10:1 are inoculated in the 24-well plate to perform co-culture for 24 to 96 hours, wherein the time when the co-culture starts is day 0; wherein, all the human cancer cells used are subjected to modification to express the reporter gene firefly luciferase. Then, the corresponding luciferase activity is measured by the fluorescence spectrophotometer at different co-culture time points, to quantify the killing degree of the differentiated macrophages against cancer cells. Please refer to FIGS. 35A-35B. 2 μg/mL Erbitux (Cetuximab), which is currently used in clinical treatment, is also added to the co-culture system of macrophages and cancer cells to further detect the killing effect of the drug against tumors, wherein the role of Erbitux is to mediate the initiation of antibody-dependent macrophage-mediated phagocytosis. FIG. 35B shows quantitative analysis result of the phagocytosis killing effect of the in vitro co-culture of human macrophage modified by immune checkpoint PD-1 fusion-based chimeric antigen receptor artificial molecular machine and PD-L1 positive human cancer cell under the mediation of Erbitux (cetuximab), and quantitative analysis line diagrams prove that the macrophages modified by immune checkpoint PD-1 fusion-based chimeric antigen receptor C #5 version have significantly different and excellent ability to identify and kill cancer cells after statistical analysis when they are co-cultured with PD-L1 positive human cancer cells, especially on day 4 (the average of C #5 group is 0.131, and the average of the control group is 0.493), while the macrophages in other experimental group C #2 and the control group do not show the ability to effectively identify and kill cancer cells when they are co-cultured with the PD-L1 positive human cancer cells; wherein, the human macrophage in the control group is the one that has not been modified by the chimeric antigen receptor artificial molecular machine, and the target cell survival index represents relative cell number of human cancer cells expressing the reporter gene firefly luciferase in the cell culture system.

Tumor Killing Detection of Macrophages Modified by Immune Checkpoint PD-1 Fusion-Based Chimeric Antigen Receptor C #3 Version or C #5 Version Against PD-L1 Positive Human Colorectal Cancer Cells in the Presence of Antibody-Dependent Cell-Mediated Phagocytosis:

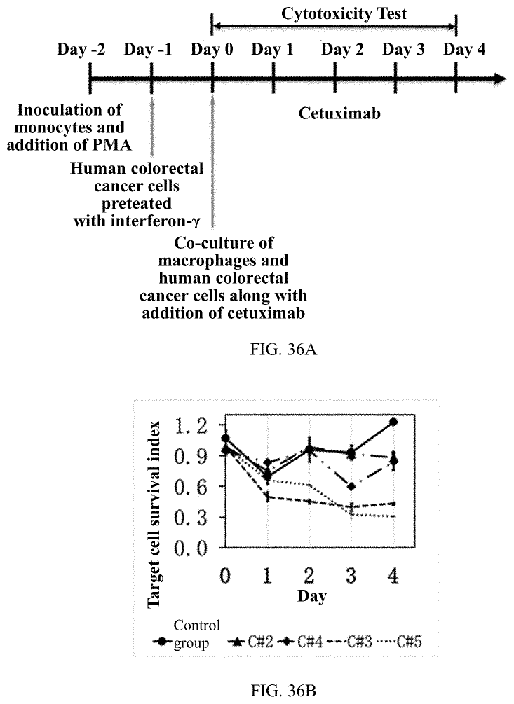

1×10⁴ modified human monocytes THP1 are first inoculated in a 24-well plate and then the phorbol ester PMA is added therein to differentiate the modified human monocytes THP1 into be macrophages; after 2 days, 1×10³ cancer cells (i.e. the modified human colorectal cancer cell DLD1 modified strain with high PD-L1 expression, which is subjected to pretreatment with 500 U/mL interferon-γ for 24 hours), at an E/T (effector cell/target cell) ratio of 10:1 are inoculated in the 24-well plate to perform co-culture for 24 to 96 hours, wherein the time when the co-culture starts is day 0; wherein, all the human cancer cells used are subjected to modification to express the reporter gene firefly luciferase. Then, the corresponding luciferase activity is measured by the fluorescence spectrophotometer at different co-culture time points, to quantify the killing degree of the differentiated macrophages against cancer cells. Please refer to FIGS. 36A-36B. 2 μg/mL Erbitux (Cetuximab), which is currently used in clinical treatment, is also added to the co-culture system of macrophages and cancer cells to further detect the killing effect of the drug against tumors, wherein the role of Erbitux is to mediate the initiation of antibody-dependent macrophage-mediated phagocytosis. FIG. 36B shows quantitative analysis result of the phagocytosis killing effect of the in vitro co-culture of human macrophage modified by immune checkpoint PD-1 fusion-based chimeric antigen receptor artificial molecular machine and PD-L1 positive human cancer cell under the mediation of Erbitux (cetuximab), and quantitative analysis line diagrams prove that the macrophages modified by immune checkpoint PD-1 fusion-based chimeric antigen receptor C #3 version or C #5 version have significantly different and excellent ability to identify and kill cancer cells after statistical analysis when they are co-cultured with PD-L1 positive human cancer cells, especially on day 4 (the average of C #3 group is 0.430, the average of C #5 group is 0.307, and the average of the control group is 1.230), while the macrophages in other experimental groups C #2 and C #4 and the control group do not show the ability to effectively identify and kill cancer cells when they are co-cultured with the PD-L1 positive human cancer cells; wherein, the human macrophage in the control group is the one that has not been modified by the chimeric antigen receptor artificial molecular machine, and the target cell survival index represents relative cell number of human cancer cells expressing the reporter gene firefly luciferase in the cell culture system.

Tumor Killing Detection of Macrophages Modified by Immune Checkpoint PD-1 Fusion-Based Chimeric Antigen Receptor C #3 Version or C #5 Version Against PD-L1 Positive Human Colorectal Cancer Cells in the Absence of Antibody-Dependent Cell-Mediated Phagocytosis:

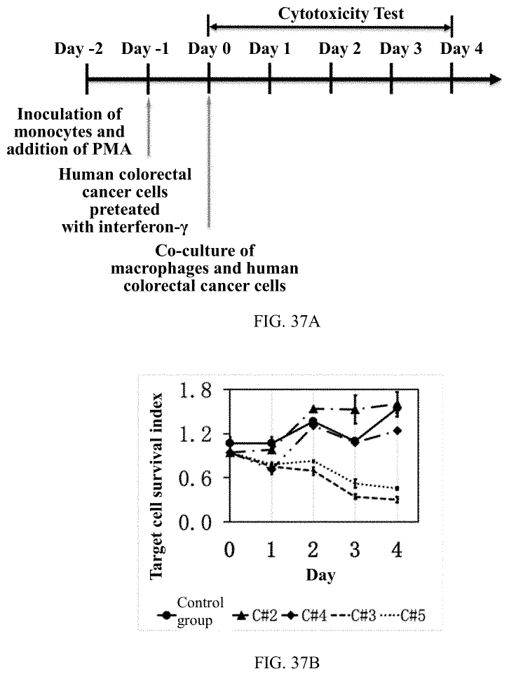

1×10⁴ modified human monocytes THP1 are first inoculated in a 24-well plate and then the phorbol ester PMA is added therein to differentiate the modified human monocytes THP1 into be macrophages; after 2 days, 1×10³ cancer cells (i.e. the modified human colorectal cancer cell DLD1 modified strain which is subjected to pretreatment with interferon-γ for 24 hours) at an E/T (effector cell/target cell) ratio of 10:1 are inoculated in the 24-well plate to perform co-culture for 24 to 96 hours, wherein the time when the co-culture starts is day 0; wherein, all the human cancer cells used are subjected to modification to express the reporter gene firefly luciferase. Then, the corresponding luciferase activity is measured by the fluorescence spectrophotometer at different co-culture time points, to quantify the killing degree of the differentiated macrophages against cancer cells. Please refer to FIGS. 37A-37B. FIG. 37B shows quantitative analysis result of the phagocytosis killing effect of the in vitro co-culture of human macrophage modified by immune checkpoint PD-1 fusion-based chimeric antigen receptor artificial molecular machine and PD-L1 positive human cancer cell, and quantitative analysis line diagrams prove that the macrophages modified by immune checkpoint PD-1 fusion-based chimeric antigen receptor C #3 version or C #5 version have significantly different and excellent ability to identify and kill cancer cells after statistical analysis when they are co-cultured with PD-L1 positive human cancer cells, especially on day 4 (the average of C #3 group is 0.301, the average of C #5 group is 0.455, and the average of the control group is 1.543), and such excellent tumor cytotoxicity could be independent of Erbitux-mediated antibody-dependent cell-mediated phagocytosis; while the macrophages in other experimental groups C #2 and C #4 and the control group do not show the ability to effectively identify and kill cancer cells when they are co-cultured with the PD-L1 positive human cancer cells; wherein, the human macrophage in the control group is the one that has not been modified by the chimeric antigen receptor artificial molecular machine, and the target cell survival index represents relative cell number of human cancer cells expressing the reporter gene firefly luciferase in the cell culture system.

Erbitux (cetuximab)-mediated antibody-dependent macrophage-mediated phagocytosis: Erbitux (cetuximab) is a therapeutic antibody targeting tumor-associated antigens, a FDA-approved therapeutic drug for treating cancer patients, and can mediate the initiation of antibody-dependent macrophage-mediated phagocytosis to identify and kill epidermal growth factor receptor (EGFR)-positive tumor target cells, such as the human colorectal cancer cell DLD1 modified strain used in the present application. The results of the in vitro tumor killing detection in the present application show that, on day 4, it can be observed that cetuximab has a certain enhancement effect in terms of the cancer cell killing effect of macrophages that have not been modified by the chimeric antigen receptor. Please see the control group with cetuximab in FIG. 36B wherein the target cell survival index is 1.230±0.016, and the control group without cetuximab in FIG. 37B wherein the target cell survival index is 1.543±0.064 and the enhancement of the killing effect is significantly different after statistical analysis. Therefore, the comprehensive results of quantitative analysis line diagrams can indicate that Erbitux-mediated antibody-dependent macrophage-mediated phagocytosis positively enhances the cancer cell killing effect of macrophages that have not been modified by the chimeric antigen receptor. In addition, from the tumor killing detection experiment of the in vitro co-culture system, it can be apparently seen the inhibitory effect of the anti-cancer drug Erbitux on cancer cells, indicating that the in vitro co-culture system used in the present application can provide clues to the effectiveness of drug treatment for sensitive tumor patients; wherein, the human macrophage in the control group is the one that has not been modified by the chimeric antigen receptor artificial molecular machine, and the target cell survival index represents relative cell number of human cancer cells expressing the reporter gene firefly luciferase in the cell culture system.

In summary, through the verification of cytotoxicity killing experiments of various tumors, the phagocytes modified by the immune checkpoint PD-1 fusion-based chimeric antigen receptor exhibit excellent phagocytosis killing ability against cancer cells, especially against PD-L1 positive human cancer cells as shown in FIGS. 3A-3D. The macrophages modified by the chimeric antigen receptor can further enhance the phagocytosis killing effect against cancer cells in the presence of antibody-dependent cell-mediated phagocytosis; wherein, the functionality of the C #3 version and C #5 version, i.e. the Truncated PD-1-Sub1-LL1-ZAP70 version and the Truncated PD-1-Sub5-LL1-SYK version, are particularly prominent. In addition, the C #4 version is an intracellular activation signaling domain mutant (ZAP70 ΔKD) of the C #3 version, that is, the intracellular activation signaling domain in the C #4 version is in a malfunctioning state. In the verification of cytotoxic killing experiments of various tumors, the phagocytes modified by C #4 version cannot effectively kill cancer cells, proving that the necessity and importance of the intracellular activation signaling domain of the chimeric antigen receptor to fully perform function for the chimeric antigen receptor.

### Example 4 PD-L1 Positive Animal Tumor Model Experiment

Taking advantage of the cross-reaction characteristics between human PD-1 and murine PD-L1 (Lázár-Molnár E et al., EBioMedicine. 2017 Mar. 1; 17:30-44.), a mouse solid tumor model with high positive expression of murine PD-L1 and well-established immune system is selected to detect and characterize the anti-tumor ability of the human immune checkpoint PD-1 fusion-based chimeric antigen receptor T cell therapy.

A mouse animal model with PD-L1 positive solid tumor and well-established immune system is established and the tumor killing effect of the human immune checkpoint PD-1 fusion-based chimeric antigen receptor artificial molecular machine modified T cell therapy.

(1) Selection of tumor target and identification of infection and expression of immune cells: in order to develop and detect the therapeutic effect of immune checkpoint (mainly PD-1)-based cell therapy, PD-L1 is selected as the tumor target, so as to detect the immunotherapy of the immune T cells modified by chimeric antigen receptor targeting PD-L1 as the target molecule in the mouse animal model with PD-L1 positive solid tumor and well-established immune system.
(2) Selection and establishment of mouse solid tumor model: B16 or MC38 is the corresponding melanoma or colon cancer cell line expressing PD-L1 which can grow into solid tumor subcutaneously in syngeneic wild-type C57BL/6 experimental mouse, is a widely used mouse PD-L1 solid tumor model, and both B16 and MC38 are PD-L1-highly expressing cancer cell that up-regulates PD-L1 expression level in response to interferon-γ (Juneja V R et al., Journal of Experimental Medicine. 2017 Apr. 3; 214(4):895-904.). The present application will use these two PD-L1-expressing solid tumor models established by being subcutaneously inoculated into wild-type mouse, and perform detection of immunotherapy of the immune T cells modified by chimeric antigen receptor targeting PD-L1 as the antigen. Thus, PD-L1-positive solid cancer cells can be recognized by the immune T cells modified by chimeric antigen receptor, thereby directly detecting the effect of cell therapy. Please refer to FIGS. 26A-26B. FIG. 26B shows the establishment, monitor and analysis flow and the treatment plan of the mouse syngeneic solid tumor model used in the present application.
(3) Package of retroviruses, infection of immune T cells and verification of the expression of chimeric antigen receptor molecular machine in immune T lymphocytes: retrovirus packaging is used to prepare viral particles of different immune checkpoint PD-1 fusion-based chimeric antigen receptor artificial molecular machines and used for subsequent infection of isolated immune T lymphocytes. The retrovirus expression vector (such as pMSCV vector) and the packaging plasmid (such as pCL-ECO viral packaging plasmid) carried with different immune checkpoint PD-1 fusion-based chimeric antigen receptor artificial molecular machines are used to transfect 293T cells, to obtain the virus supernatant which is then subjected to filtration, subpackaging and freezing to determine the virus titer. Primary mouse T lymphocytes from peripheral lymph nodes and spleen are isolated from wild-type donor mouse using a commercial mouse T lymphocyte isolation kit (such as the German Miltenyi Mouse T Lymphocyte Isolation Magnetic Bead Kit). The primary mouse T lymphocytes are then cultured and stimulated with anti-CD3/anti-CD28-coated multi-well culture dish for 24 hours, and then a certain amount of viruses are added therein for infection. After infection of 24 to 72 hours, the expression level of the chimeric antigen receptor on the surface of the modified primary T cells is detected by flow staining of antibody and continue to culture and expand the primary T cells in vitro for use in animal experiments. Additionally, the corresponding retroviral multiplicity of infection (MOI) can be optimized to support subsequent experiments. In the process of virus infection, fresh culture medium needs to be supplemented in time. Please refer to FIG. 26A. FIG. 26A shows the in vitro isolation, infection and expansion process of the lymphatic T cell of the donor mouse used in the present application.
(4) Anti-tumor effect experiment of T cell therapy modified by chimeric antigen receptor molecular machine in animal solid tumor model:

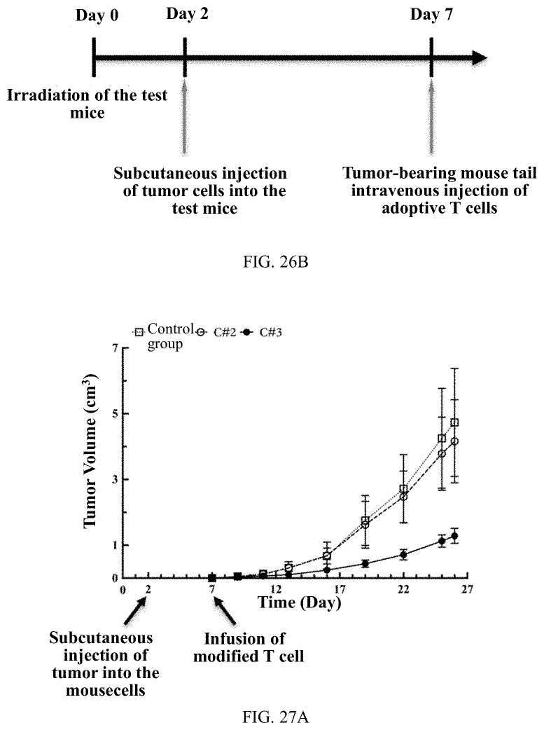

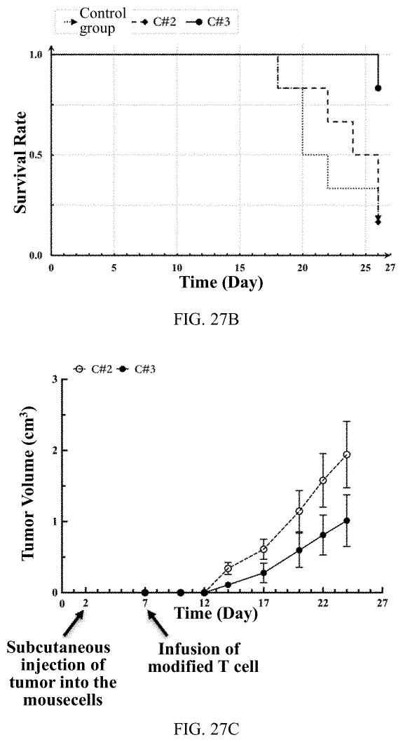

The experimental mice are irradiated (non-lethal dose, 3-5 Gy irradiation dose) 2 days before the subcutaneous injection of cancer cells (recorded as day 0) to achieve the clearing of peripheral blood lymphocytes of the experimental mice. Then, on day 2, 2×10⁵-20×10⁵ PD-L1 positive B16 or MC38 cells are inoculated into the back of the experimental mice subcutaneously to establish the mouse animal model with PD-L1 positive solid tumor and well-established immune system. The tumor growth size is continuously measured from day 5 after the subcutaneous inoculation of cancer cells in the experimental mice, the tumor-bearing mice are divided into groups and adoptively infused with different T cell subsets (such as immunogenic primary CD8-positive T lymphocytes that are modified by immune checkpoint fusion-based chimeric antigen receptor or that are not modified by immune checkpoint fusion-based chimeric antigen receptor) by tail vein injection, and the tumor size and survival rate of the mice are regularly detected. Please refer to FIG. 26B and FIGS. 27A-27C. FIG. 26B shows the establishment, monitor and analysis flow and the treatment plan of the mouse syngeneic solid tumor model used in the present application. FIG. 27A shows quantitative analysis of treatment effects of different immune checkpoint PD-1 fusion-based chimeric antigen receptor artificial molecular machine-modified T cell therapies in a mouse animal model with PD-L1 positive melanoma solid tumor and the well-established immune system.

Quantitative analysis line diagrams in FIG. 27A prove that the T cells modified by chimeric antigen receptor C #3 have significantly different and excellent anti-cancer ability to identify and kill cancer cells after statistical analysis in the mouse animal model with PD-L1 positive murine melanoma solid tumor, while the T cells in other experimental group C #2 and the control group do not show the anti-cancer ability to effectively identify and kill cancer cells in the mouse animal model with PD-L1 positive murine melanoma solid tumor. Please refer to FIG. 28 and related description according to the present application for the information of each component included in the immune checkpoint PD-1 fusion-based chimeric antigen receptor C #2 version and C #3 version; wherein, the T cell therapy in the control group refers to use of murine immunogenic primary T cell that has not been modified by chimeric antigen receptor artificial molecular machine, and the tumor volume represents the quantitative volume of solid tumor in the mouse subcutaneous solid tumor model, and the mouse tumor model is a subcutaneous B16 melanoma solid tumor model. Please refer to FIGS. 26A-26B for the specific treatment plan flow information.

FIG. 27B shows quantitative analysis of treatment effects of different immune checkpoint PD-1 fusion-based chimeric antigen receptor artificial molecular machine-modified T cell therapies in the mouse animal model with PD-L1 positive melanoma solid tumor and the well-established immune system.

Quantitative analysis line diagrams in FIG. 27B prove that the T cells modified by chimeric antigen receptor C #3 have significantly different and excellent anti-cancer effect to prolong the survival cycle of tumor-bearing mice and improve the survival rate of tumor-bearing mice in the mouse animal model with PD-L1 positive murine melanoma solid tumor, while the T cells in other experimental group C #2 and the control group do not show the anti-cancer effect to effectively prolong the survival cycle of tumor-bearing mice and improve the survival rate of tumor-bearing mice in the mouse animal model with PD-L1 positive murine melanoma solid tumor. Please refer to FIG. 28 and related description according to the present application for the information of each component included in the immune checkpoint PD-1 fusion-based chimeric antigen receptor C #2 version and C #3 version; wherein, the T cell therapy in the control group refers to use of murine immunogenic primary T cell that have not been modified by chimeric antigen receptor artificial molecular machine, the ordinate of the survival curve refers to the survival rate, the abscissa refers to the survival time, and the mouse tumor model is a subcutaneous B16 melanoma solid tumor model. Please refer to FIGS. 26A-26B for the specific treatment plan flow information.

FIG. 27C shows quantitative analysis of treatment effects of different immune checkpoint PD-1 fusion-based chimeric antigen receptor artificial molecular machine-modified T cell therapies in a mouse animal model with PD-L1 positive colon cancer solid tumor and a well-established immune system.

Quantitative analysis line diagrams in FIG. 27C prove that the T cells modified by chimeric antigen receptor C #3 have significantly different and excellent anti-cancer ability to identify and kill cancer cells after statistical analysis in the mouse animal model with PD-L1 positive colon cancer solid tumor, while the T cells in other experimental group C #2 do not show the anti-cancer ability to effectively identify and kill cancer cells in the mouse animal model with PD-L1 positive colon cancer solid tumor. Please refer to FIG. 28 and related description according to the present application for the information of each component included in the immune checkpoint PD-1 fusion-based chimeric antigen receptor C #2 version and C #3 version; wherein, the tumor volume represents the quantitative volume of the solid tumor in the mouse subcutaneous solid tumor model, and the mouse tumor model is a subcutaneous MC38 colon cancer solid tumor model. Please refer to FIGS. 26A-26B for the specific treatment plan flow information.

In summary, the experimental results of the solid tumor mouse animal model show that the adoptive therapy of T lymphocytes based on the C #3 version exhibit significant effect to inhibit the growth of PD-L1 tumor, while the other control groups do not show an anti-tumor effect, indicating that the T-cell adoptive therapy modified by version C #3 has a good anti-PD-L1-expressing tumor effect, and significantly improves the survival rate of the corresponding tumor-bearing mice.

Finally, as mentioned above, immune checkpoint blocker and cell therapy make major breakthroughs in the field of tumor immunity recently. Although CAR-T and other cell therapies have achieved exciting results in the treatment of blood cancers, their role in the treatment of solid tumors remains to be further explored. Considering the advantages and disadvantages of PD-1/PD-L1 antibody drugs and cell therapy (such as CAR-T)-based drugs, the present application combines various means such as tumor immunology, synthetic biology, and molecular cell engineering to develop a new generation of solid cancer cell therapy based on the immune checkpoint PD-1/PD-L1 signaling pathway, and has both advantages thereof. The cell therapy uses an immune checkpoint-based chimeric antigen receptor molecular machine having an immune cell regulation coding function. When the cancer cells expressing the immune checkpoint inhibitory signal PD-L1 as PD-1 molecule ligand try to inhibit the function of immune T cells or phagocytes by means of the same brake blocking mechanism to immune cells in the immune checkpoint PD-1/PD-L1 signaling pathway, the immune T cells or phagocytes modified by re-encoding with this new generation of PD-1-based chimeric antigen receptor molecular machine will not be inhibited by cancer cells, but will be further activated, generating specific immune response against the corresponding cancer cells, thereby more effectively identifying and killing the corresponding cancer cells.

The chimeric antigen receptor molecular machine modified immune cells in the present application, including immune T cells or various phagocytes, can better present activation ability of corresponding immune cells and achieve the ability to kill and clear a variety of tumors highly expressing PD-L1 (such as breast cancer, colorectal cancer, skin cancer, colon cancer, pancreatic cancer, liver cancer, ovarian cancer, prostate cancer, brain cancer, kidney cancer, lung cancer, lymphoma cancer and melanoma), which are proved by extracellular experiments, intracellular experiments and animal tumor model experiments with well-established immune system. The efficacy of immune cells modified by the chimeric antigen receptor in clearing solid tumors is much higher than that the current FDA-authorized PD-1 immune checkpoint inhibitors—Opdivo (i.e. Opdivo, Nivolumab) and Keytruda (i.e. Pembrolizumab), and also overcomes immunosuppression in the solid tumor microenvironment, which is a key problem in solid tumor immunotherapy. Therefore, the immune cells modified by chimeric antigen receptor molecular machine successfully overcome the immunosuppression in the solid tumor microenvironment, that is, solve the key problem in the solid tumor immunotherapy. It is believed that such tools could open new approaches to solid tumor treatment and provide innovative and precise therapies for human cancer treatment.

The above examples are only illustrative, and do not limit the present application in any form. Any change or modification, made by the skilled in the art based on the technical content disclosed above, without departing from the spirit of the present application, is equivalent example and falls within the scope of the present application.

What is claimed is:

1. A chimeric antigen receptor, comprising:
a) an extracellular target molecule binding domain for specifically binding to a target molecule;
b) an intracellular signaling domain comprising at least one intracellular activation signaling domain and/or at least one intracellular detection signaling domain; and
c) a transmembrane domain, used to connect the extracellular target molecule binding domain and the intracellular signaling domain, and to anchor the extracellular target molecule binding domain and the intracellular signaling domain on a cell membrane;
wherein, an activation of the intracellular activation signaling domain at least relies on a binding of the extracellular target molecule binding domain to the target molecule; and the intracellular activation signaling domain comprises a molecule or a fragment comprising a catalytic domain.

2. The chimeric antigen receptor according to claim 1, wherein the intracellular activation signaling domain comprises at least one of receptor-type tyrosine kinase, receptor-type tyrosine kinase fragment, non-receptor-type tyrosine kinase, and non-receptor-type tyrosine kinase fragment;
preferably, the receptor-type tyrosine kinase is at least one of EGFR, HER2, HER3, HER4, InsR, IGF1R, IRR, PDGFRα, PDGFRβ, Kit, CSFR, FLT3, VEGFR-1, VEGFR-2, VEGFR-3, FGFR1, FGFR2, FGFR3, FGFR4, CCK4, trkA, trkB, trkC, ROR1, ROR2, MuSK, MET, Ron, Axl, Tyro3, Mer, TIE1, TIE2, EphA1, EphA2, EphA3, EphA4, EphA5, EphA6, EphA7, EphA8, EphA10, EphB1, EphB2, EphB3, EphB4, EphB6, Ret, RYK, DDR1, DDR2, ROS, Lmr1, Lmr2, Lmr3, LTK, ALK, and STYK1; and the non-receptor tyrosine kinase is at least one of Abl, Arg, Tnk1, Ack, CSK, CTK, FAK, Pyk2, Fer, Fes, JAK1, JAK2, JAK3, Tyk2, Blk, Fgr, FRK, Fyn, Hck, Lck, Lyn, Brk, Src, Srm, Yes, Syk, ZAP70, Etk, Btk, ITK, TEC, and TXK;
preferably, the intracellular activation signaling domain comprises at least one of an amino acid sequence comprising SEQ ID NO: 042, an amino acid sequence comprising SEQ ID NO: 044, an amino acid sequence comprising SEQ ID NO: 046, an amino acid sequence comprising SEQ ID NO: 048, an amino acid sequence comprising SEQ ID NO: 050, and an amino acid sequence comprising SEQ ID NO: 052.

3-4. (canceled)

5. The chimeric antigen receptor according to claim 1, wherein the intracellular detection signaling domain comprises at least one immunoreceptor tyrosine-based activation motif ITAM;
preferably, the intracellular detection signaling domain is at least one of CD3ζ ITAM1 fragment, CD3ζ ITAM2 fragment, CD3ζ ITAM3 fragment, FcRIIA ITAM fragment, FcRγ ITAM fragment, DAP12 ITAM fragment, and CD3ε ITAM fragment;
preferably, the intracellular detection signaling domain comprises at least one signaling domain of a molecule selected from 2B4, CD244, BTLA, CD3δ, CD3γ, CD3ε, CD3ζ, CD5, CD28, CD31, CD72, CD84, CD229, CD300a, CD300f, CEACAM-1, CEACAM-3, CEACAM-4, CEACAM-19, CEACAM-20, CLEC-1, CLEC-2, CRACC, CTLA-4, DAP10, DAP12, DCAR, DCIR, Dectin-1, DNAM-1, FcεRIα, FcεRIβ, FcγRIB, FcγRI, FcγRIIA, FcγRIIB, FcγRIIC, FcγRIIIA, FCRL1, FCRL2, FCRL3, FCRL4, FCRL5, FCRL6, G6b, KIR, KIR2DL1, KIR2DL2, KIR2DL3, KIR2DL4, KIR2DL5, KIR2DL5B, KIR2DS1, KIR2DS3, KIR2DS4, KIR2DS5, KIR3DL1, KIR3DL2, KIR3DL3, KIR3DS1, KLRG1, LAIR1, LILRB1, LILRB2, LILRB3, LILRB4, LILRB5, MICL, NKG2A, NKp44, NKp65, NKp80, NTB-A, PD-1, PDCD6, PILR-α, Siglec-2, Siglec-3, Siglec-5, Siglec-6, Siglec-7, Siglec-8, Siglec-9, Siglec-10, Siglec-11, Siglec-12, Siglec-14, Siglec-15, Siglec-16, SIRPα, SLAM, TIGIT, TREML1, and TREML2,
preferably, the intracellular detection signaling domain comprises at least one of an amino acid sequence comprising SEQ ID NO: 020, an amino acid sequence comprising SEQ ID NO: 022, an amino acid sequence comprising SEQ ID NO: 024, an amino acid sequence comprising SEQ ID NO: 026, an amino acid sequence comprising SEQ ID NO: 028, an amino acid sequence comprising SEQ ID NO: 030, an amino acid sequence comprising SEQ ID NO: 032, an amino acid sequence comprising SEQ ID NO: 034, an amino acid sequence comprising SEQ ID NO: 036, an amino acid sequence comprising SEQ ID NO: 038, and an amino acid sequence comprising SEQ ID NO: 040.

6-8. (canceled)

9. The chimeric antigen receptor according to claim 1, wherein the target molecule bound by the extracellular target molecule binding domain comprises at least one of the following molecules: immunosuppressive signal-related molecule, tumor surface antigen molecular marker, and specific antigen peptide-histocompatibility complex molecule on cell surface;
preferably, the extracellular target molecule binding domain comprises at least one of target molecule binding domain of a molecule selected from PD-1, truncated PD-1, PD-1 variant, antibody against PD-L1, and PD-L1-binding fragment;
preferably, the extracellular target molecule binding domain comprises at least one of an amino acid sequence comprising SEQ ID NO: 001, an amino acid sequence comprising SEQ ID NO: 003, an amino acid sequence comprising SEQ ID NO: 005, an amino acid sequence comprising SEQ ID NO: 007, an amino acid sequence comprising SEQ ID NO: 009, and an amino acid sequence comprising SEQ ID NO: 011.

10-11. (canceled)

12. The chimeric antigen receptor according to claim 1, wherein the transmembrane domain is at least one transmembrane domain of a transmembrane protein selected from PD-1, PD-L1, PD-L2, 4-1BB, 4-1BBL, ICOS, GITR, GITRL, OX40, OX40L, CD40, CD40L, CD86, CD80, CD2, CD28, B7-DC, B7-H2, B7-H3, B7-H4, B7-H5, B7-H6, B7-H7, VSIG-3, VISTA, SIRPα, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, Siglec-15, Siglec-16, DAP10, DAP12, NKG2A, NKG2C, NKG2D, LIR1, KIR, KIR2DL1, KIR2DL2, KIR2DL3, KIR2DL4, KIR2DL5A, KIR2DL5B, KIR2DS1, KIR2DS3, KIR2DS4, KIR2DS5, KIR3DL1, KIR3DL2, KIR3DL3, KIR3DS1, KLRG1, KLRG2, LAIR1, LAIR2, LILRA1, LILRA2, LILRA3, LILRA4, LILRA5, LILRB1, LILRB2, LILRB3, LILRB4, LILRB5, 2B4, BTLA, CD160, LAG-3, CTLA-4, CD155, CD112, CD113, TIGIT, CD96, CD226, TIM-1, TIM-3, TIM-4, Galectin-9, CEACAM-1, CD8a, CD8b, CD4, MERTK, AXL, Tyro3, BAIL MRC1, MRC2, FcγR1, FcγR2A, FcγR2B1, FcγR2B2, FcγR2C, FcγR3A, FcγR3B, FcεR2, FcεR1, FcRn, Fcα/μR, and FcαR1;
preferably, the transmembrane domain comprises at least one of an amino acid sequence comprising SEQ ID NO: 012 and an amino acid sequence comprising SEQ ID NO: 014.

13. (canceled)

14. The chimeric antigen receptor according to claim 1, wherein the intracellular detection signaling domain is connected to the intracellular activation signaling domain, and the intracellular detection signaling domain is located between the transmembrane domain and the intracellular activation signaling domain.

15. The chimeric antigen receptor according to claim 1, wherein an extracellular spacer domain is further included between the extracellular target molecule binding domain and the transmembrane domain;
preferably, the extracellular spacer domain comprises at least one of an amino acid sequence comprising SEQ ID NO: 016 and an amino acid sequence comprising SEQ ID NO: 018.

16. (canceled)

17. The chimeric antigen receptor according to claim 1, wherein the chimeric antigen receptor further comprises an intracellular spacer domain, and the intracellular spacer domain is located between the transmembrane domain and the intracellular signaling domain and connects the transmembrane domain and the intracellular signaling domain together;
preferably, the intracellular spacer domain is an extension of the transmembrane domain, comprising at least one molecule selected from PD-1, PD-L1, PD-L2, 4-1BB, 4-1BBL, ICOS, GITR, GITRL, OX40, OX40L, CD40, CD40L, CD86, CD80, CD2, CD28, B7-DC, B7-H2, B7-H3, B7-H4, B7-H5, B7-H6, B7-H7, VSIG-3, VISTA, SIRPα, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, Siglec-15, Siglec-16, DAP10, DAP12, NKG2A, NKG2C, NKG2D, LIR1, KIR, KIR2DL1, KIR2DL2, KIR2DL3, KIR2DL4, KIR2DL5A, KIR2DL5B, KIR2DS1, KIR2DS3, KIR2DS4, KIR2DS5, KIR3DL1, KIR3DL2, KIR3DL3, KIR3DS1, KLRG1, KLRG2, LAIR1, LAIR2, LILRA1, LILRA2, LILRA3, LILRA4, LILRA5, LILRB1, LILRB2, LILRB3, LILRB4, LILRB5, 2B4, BTLA, CD160, LAG-3, CTLA-4, CD155, CD112, CD113, TIGIT, CD96, CD226, TIM-1, TIM-3, TIM-4, Galectin-9, CEACAM-1, CD8a, CD8b, CD4, MERTK, AXL, Tyro3, BAIL MRC1, MRC2, FcγR1, FcγR2A, FcγR2B1, FcγR2B2, FcγR2C, FcγR3A, FcγR3B, FcεR2, FcεR1, FcRn, Fcα/μR, and FcαR1;
preferably, the intracellular spacer domain comprises at least one of an amino acid sequence comprising SEQ ID NO: 054 and an amino acid sequence comprising SEQ ID NO: 056.

18-19. (canceled)

20. The chimeric antigen receptor according to claim 1, wherein the chimeric antigen receptor further comprises an intracellular linker domain, and the intracellular linker domain connects the intracellular detection signaling domain with the intracellular activation signaling domain;
preferably, the intracellular linker domain comprises at least one of an amino acid sequence comprising SEQ ID NO: 058, an amino acid sequence comprising SEQ ID NO: 060, an amino acid sequence comprising SEQ ID NO: 062, an amino acid sequence comprising SEQ ID NO: 064, and an amino acid sequences comprising SEQ ID NO: 066.

21. (canceled)

22. The chimeric antigen receptor according to claim 1, wherein the chimeric antigen receptor is an immune cell chimeric antigen receptor.

23. The chimeric antigen receptor according to claim 22, wherein the immune cell comprises T lymphocyte;
preferably, the T lymphocyte comprises at least one of inflammatory T lymphocyte, cytotoxic T lymphocyte, regulatory T lymphocyte, and helper T lymphocyte;
preferably, the T lymphocyte comprises at least one of CD4⁺ T lymphocyte, CD8⁺ T lymphocyte, γδ T lymphocyte, and NKT lymphocyte.

24-25. (canceled)

26. The chimeric antigen receptor according to claim 22, wherein the immune cell comprises phagocyte;
preferably, the phagocyte comprises at least one of macrophage, monocyte, neutrophil, mast cell, dendritic cell, and B cell.

27. (canceled)

28. The chimeric antigen receptor according to claim 1 comprising
a) the extracellular target molecule binding domain comprising at least one of an amino acid sequence comprising SEQ ID NO: 001, an amino acid sequence comprising SEQ ID NO: 003, an amino acid sequence comprising SEQ ID NO: 005, an amino acid sequence comprising SEQ ID NO: 007, an amino acid sequence comprising SEQ ID NO: 009, and an amino acid sequence comprising SEQ ID NO: 011;
b) the transmembrane domain comprising at least one of an amino acid sequence comprising SEQ ID NO: 012 and an amino acid sequence comprising SEQ ID NO: 014;
c) an extracellular spacer domain through which the extracellular target molecule binding domain and the transmembrane domain are connected, wherein the extracellular spacer domain comprises at least one of an amino acid sequence comprising SEQ ID NO: 016 and an amino acid sequence comprising SEQ ID NO: 018; and
d) the intracellular signaling domain comprising at least one of an amino acid sequence comprising SEQ ID NO: 020, an amino acid sequence comprising SEQ ID NO: 022, an amino acid sequence comprising SEQ ID NO: 024, an amino acid sequence comprising SEQ ID NO: 026, an amino acid sequence comprising SEQ ID NO: 028, an amino acid sequence comprising SEQ ID NO: 030, an amino acid sequence comprising SEQ ID NO: 032, an amino acid sequence comprising SEQ ID NO: 034, an amino acid sequence comprising SEQ ID NO: 036, an amino acid sequence comprising SEQ ID NO: 038, an amino acid sequence comprising SEQ ID NO: 040, an amino acid sequence comprising SEQ ID NO: 042, an amino acid sequence comprising SEQ ID NO: 044, an amino acid sequence comprising SEQ ID NO: 046, an amino acid sequence comprising SEQ ID NO: 048, an amino acid sequence comprising SEQ ID NO: 050, and an amino acid sequence comprising SEQ ID NO: 052;
or
the chimeric antigen receptor comprising
a) the extracellular target molecule binding domain comprising at least one of an amino acid sequence comprising SEQ ID NO: 001, an amino acid sequence comprising SEQ ID NO: 003, an amino acid sequence comprising SEQ ID NO: 005, an amino acid sequence comprising SEQ ID NO: 007, an amino acid sequence comprising SEQ ID NO: 009, and an amino acid sequence comprising SEQ ID NO: 011;
b) the transmembrane domain comprising at least one of an amino acid sequence comprising SEQ ID NO: 012 and an amino acid sequence comprising SEQ ID NO: 014;
c) an extracellular spacer domain through which the extracellular target molecule binding domain and the transmembrane domain are connected, wherein the extracellular spacer domain comprises at least one of an amino acid sequence comprising SEQ ID NO: 016 and an amino acid sequence comprising SEQ ID NO: 018; and
d) the intracellular activation signaling domain comprising at least one of an amino acid sequence comprising SEQ ID NO: 042, an amino acid sequence comprising SEQ ID NO: 044, an amino acid sequence comprising SEQ ID NO: 046, an amino acid sequence comprising SEQ ID NO: 048, an amino acid sequence comprising SEQ ID NO: 050, and an amino acid sequence comprising SEQ ID NO: 052;
or
the chimeric antigen receptor comprising
a) the extracellular target molecule binding domain comprising at least one of an amino acid sequence comprising SEQ ID NO: 001, an amino acid sequence comprising SEQ ID NO: 003, an amino acid sequence comprising SEQ ID NO: 005, an amino acid sequence comprising SEQ ID NO: 007, an amino acid sequence comprising SEQ ID NO: 009, and an amino acid sequence comprising SEQ ID NO: 011;
b) the transmembrane domain comprising at least one of an amino acid sequence comprising SEQ ID NO: 012 and an amino acid sequence comprising SEQ ID NO: 014;
c) an extracellular spacer domain through which the extracellular target molecule binding domain and the transmembrane domain are connected, wherein the extracellular spacer domain comprises at least one of an amino acid sequence comprising SEQ ID NO: 016 and an amino acid sequence comprising SEQ ID NO: 018;
d) the intracellular detection signaling domain comprising at least one of an amino acid sequence comprising SEQ ID NO: 020, an amino acid sequence comprising SEQ ID NO: 022, an amino acid sequence comprising SEQ ID NO: 024, an amino acid sequence comprising SEQ ID NO: 026, an amino acid sequence comprising SEQ ID NO: 028, an amino acid sequence comprising SEQ ID NO: 030, an amino acid sequence comprising SEQ ID NO: 032, an amino acid sequence comprising SEQ ID NO: 034, an amino acid sequence comprising SEQ ID NO: 036, an amino acid sequence comprising SEQ ID NO: 038, and an amino acid sequence comprising SEQ ID NO: 040; and
e) the intracellular activation signaling domain comprising at least one selected from the group consisting of an amino acid sequence comprising SEQ ID NO: 042, an amino acid sequence comprising SEQ ID NO: 044, an amino acid sequence comprising SEQ ID NO: 046, an amino acid sequence comprising SEQ ID NO: 048, an amino acid sequence comprising SEQ ID NO: 050, and an amino acid sequence comprising SEQ ID NO: 052;
or
the chimeric antigen receptor comprising
a) the extracellular target molecule binding domain comprising at least one of an amino acid sequence comprising SEQ ID NO: 001, an amino acid sequence comprising SEQ ID NO: 003, an amino acid sequence comprising SEQ ID NO: 005, an amino acid sequence comprising SEQ ID NO: 007, an amino acid sequence comprising SEQ ID NO: 009, and an amino acid sequence comprising SEQ ID NO: 011;
b) the transmembrane domain comprising at least one of an amino acid sequence comprising SEQ ID NO: 012 and an amino acid sequence comprising SEQ ID NO: 014;
c) an extracellular spacer domain through which the extracellular target molecule binding domain and the transmembrane domain are connected, wherein the extracellular spacer domain comprises at least one of an amino acid sequence comprising SEQ ID NO: 016 and an amino acid sequence comprising SEQ ID NO: 018;
d) the intracellular detection signaling domain comprising at least one of an amino acid sequence comprising SEQ ID NO: 020, an amino acid sequence comprising SEQ ID NO: 022, an amino acid sequence comprising SEQ ID NO: 024, an amino acid sequence comprising SEQ ID NO: 026, an amino acid sequence comprising SEQ ID NO: 028, an amino acid sequence comprising SEQ ID NO: 030, an amino acid sequence comprising SEQ ID NO: 032, an amino acid sequence comprising SEQ ID NO: 034, an amino acid sequence comprising SEQ ID NO: 036, an amino acid sequence comprising SEQ ID NO: 038, and an amino acid sequence comprising SEQ ID NO: 040;
e) the intracellular activation signaling domain comprising at least one of an amino acid sequence comprising SEQ ID NO: 042, an amino acid sequence comprising SEQ ID NO: 044, an amino acid sequence comprising SEQ ID NO: 046, an amino acid sequence comprising SEQ ID NO: 048, an amino acid sequence comprising SEQ ID NO: 050, and an amino acid sequence comprising SEQ ID NO: 052; and
f) an intracellular linker domain through which the intracellular detection signaling domain and the intracellular activation signaling domain are connected, wherein the intracellular linker domain comprises at least one of an amino acid sequence comprising SEQ ID NO: 058, an amino acid sequence comprising SEQ ID NO: 060, an amino acid sequence comprising SEQ ID NO: 062, an amino acid sequence comprising SEQ ID NO: 064, and an amino acid sequence comprising SEQ ID NO: 066;
or
the chimeric antigen receptor comprising
a) the extracellular target molecule binding domain comprising at least one of an amino acid sequence comprising SEQ ID NO: 001, an amino acid sequence comprising SEQ ID NO: 003, an amino acid sequence comprising SEQ ID NO: 005, an amino acid sequence comprising SEQ ID NO: 007, an amino acid sequence comprising SEQ ID NO: 009, and an amino acid sequence comprising SEQ ID NO: 011;
b) the transmembrane domain comprising at least one of an amino acid sequence comprising SEQ ID NO: 012 and an amino acid sequence comprising SEQ ID NO: 014;
c) an extracellular spacer domain through which the extracellular target molecule binding domain and the transmembrane domain are connected, wherein the extracellular spacer domain comprises at least one of an amino acid sequence comprising SEQ ID NO: 016 and an amino acid sequence comprising SEQ ID NO: 018;
d) an intracellular signaling domain comprising at least one of an amino acid sequence comprising SEQ ID NO: 020, an amino acid sequence comprising SEQ ID NO: 022, an amino acid sequence comprising SEQ ID NO: 024, an amino acid sequence comprising SEQ ID NO: 026, an amino acid sequence comprising SEQ ID NO: 028, an amino acid sequence comprising SEQ ID NO: 030, an amino acid sequence comprising SEQ ID NO: 032, an amino acid sequence comprising SEQ ID NO: 034, an amino acid sequence comprising SEQ ID NO: 036, an amino acid sequence comprising SEQ ID NO: 038, an amino acid sequence comprising SEQ ID NO: 040, an amino acid sequence comprising SEQ ID NO: 042, an amino acid sequence comprising SEQ ID NO: 044, an amino acid sequence comprising SEQ ID NO: 046, an amino acid sequence comprising SEQ ID NO: 048, an amino acid sequence comprising SEQ ID NO: 050, and an amino acid sequence comprising SEQ ID NO: 052; and
e) an intracellular spacer domain through which the transmembrane domain and the intracellular signaling domain are connected, wherein the intracellular spacer domain comprises at least one of an amino acid sequence comprising SEQ ID NO: 054 and an amino acid sequence comprising SEQ ID NO: 056;
or
the chimeric antigen receptor comprising
a) an extracellular target molecule binding domain comprising at least one of an amino acid sequence comprising SEQ ID NO: 001, an amino acid sequence comprising SEQ ID NO: 003, an amino acid sequence comprising SEQ ID NO:005, an amino acid sequence comprising SEQ ID NO:007, an amino acid sequence comprising SEQ ID NO:009, and an amino acid sequence comprising SEQ ID NO:011;
b) the transmembrane domain comprising at least one of an amino acid sequence comprising SEQ ID NO: 012 and an amino acid sequence comprising SEQ ID NO: 014;
c) an extracellular spacer domain through which the extracellular target molecule binding domain and the transmembrane domain are connected, wherein the extracellular spacer domain comprises at least one of an amino acid sequence comprising SEQ ID NO: 016 and an amino acid sequence comprising SEQ ID NO: 018; and
d) an intracellular activation signaling domain comprising at least one of an amino acid sequence comprising SEQ ID NO: 042, an amino acid sequence comprising SEQ ID NO: 044, an amino acid sequence comprising SEQ ID NO: 046, an amino acid sequence comprising SEQ ID NO: 048, an amino acid sequence comprising SEQ ID NO: 050, and an amino acid sequence comprising SEQ ID NO: 052; and
e) an intracellular spacer domain through which the transmembrane domain and the intracellular activation signaling domain are connected, wherein the intracellular spacer domain comprises at least one of an amino acid sequence comprising SEQ ID NO: 054 and an amino acid sequence comprising SEQ ID NO: 056;
or
the chimeric antigen receptor comprising
a) the extracellular target molecule binding domain comprising at least one of an amino acid sequence comprising SEQ ID NO: 001, an amino acid sequence comprising SEQ ID NO: 003, an amino acid sequence comprising SEQ ID NO: 005, an amino acid sequence comprising SEQ ID NO: 007, an amino acid sequence comprising SEQ ID NO: 009, and an amino acid sequence comprising SEQ ID NO: 011;
b) the transmembrane domain comprising at least one of an amino acid sequence comprising SEQ ID NO: 012 and an amino acid sequence comprising SEQ ID NO: 014;
c) an extracellular spacer domain through which the extracellular target molecule binding domain and the transmembrane domain are connected, wherein the extracellular spacer domain comprises at least one of an amino acid sequence comprising SEQ ID NO: 016 and an amino acid sequence comprising SEQ ID NO: 018;
d) the intracellular detection signaling domain comprising at least one of an amino acid sequence comprising SEQ ID NO: 020, an amino acid sequence comprising SEQ ID NO: 022, an amino acid sequence comprising SEQ ID NO: 024, an amino acid sequence comprising SEQ ID NO: 026, an amino acid sequence comprising SEQ ID NO: 028, an amino acid sequence comprising SEQ ID NO: 030, an amino acid sequence comprising SEQ ID NO: 032, an amino acid sequence comprising SEQ ID NO: 034, an amino acid sequence comprising SEQ ID NO: 036, an amino acid sequence comprising SEQ ID NO: 038 and an amino acid sequence comprising SEQ ID NO: 040;
e) an intracellular activation signaling domain comprising at least one of an amino acid sequence comprising SEQ ID NO: 042, an amino acid sequence comprising SEQ ID NO: 044, an amino acid sequence comprising SEQ ID NO: 046, an amino acid sequence comprising SEQ ID NO: 048, an amino acid sequence comprising SEQ ID NO: 050, and an amino acid sequence comprising SEQ ID NO: 052; and
f) an intracellular spacer domain through which the transmembrane domain and the intracellular detection signaling domain are connected, wherein the intracellular spacer domain comprises at least one of an amino acid sequence comprising SEQ ID NO: 054 and an amino acid sequence comprising SEQ ID NO: 056;
or
the chimeric antigen receptor comprising
a) the extracellular target molecule binding domain comprising at least one of an amino acid sequence comprising SEQ ID NO: 001, an amino acid sequence comprising SEQ ID NO: 003, an amino acid sequence comprising SEQ ID NO: 005, an amino acid sequence comprising SEQ ID NO: 007, an amino acid sequence comprising SEQ ID NO: 009, and an amino acid sequence comprising SEQ ID NO: 011;
b) the transmembrane domain comprising at least one of an amino acid sequence comprising SEQ ID NO: 012 and an amino acid sequence comprising SEQ ID NO: 014;
c) an extracellular spacer domain through which the extracellular target molecule binding domain and the transmembrane domain are connected, wherein the extracellular spacer domain comprises at least one of an amino acid sequence comprising SEQ ID NO: 016 and an amino acid sequence comprising SEQ ID NO: 018;
d) the intracellular detection signaling domain comprising at least one of an amino acid sequence comprising SEQ ID NO: 020, an amino acid sequence comprising SEQ ID NO: 022, an amino acid sequence comprising SEQ ID NO: 024, an amino acid sequence comprising SEQ ID NO: 026, an amino acid sequence comprising SEQ ID NO: 028, an amino acid sequence comprising SEQ ID NO: 030, an amino acid sequence comprising SEQ ID NO: 032, an amino acid sequence comprising SEQ ID NO: 034, an amino acid sequence comprising SEQ ID NO: 036, an amino acid sequence comprising SEQ ID NO: 038, and an amino acid sequence comprising SEQ ID NO:040;
e) an intracellular activation signaling domain comprising at least one of an amino acid sequence comprising SEQ ID NO: 042, an amino acid sequence comprising SEQ ID NO: 044, an amino acid sequence comprising SEQ ID NO: 046, an amino acid sequence comprising SEQ ID NO: 048, an amino acid sequence comprising SEQ ID NO: 050, and an amino acid sequence comprising SEQ ID NO: 052;
f) an intracellular spacer domain through which the transmembrane domain and the intracellular detection signaling domain are connected, wherein the intracellular spacer domain comprises at least one of an amino acid sequence comprising SEQ ID NO: 054 and an amino acid sequence comprising SEQ ID NO: 056; and
g) an intracellular linker domain through which the intracellular detection signaling domain and the intracellular activation signaling domain are connected, wherein the intracellular linker domain comprises at least one of an amino acid sequence comprising SEQ ID NO: 058, an amino acid sequence comprising SEQ ID NO: 060, an amino acid sequence comprising SEQ ID NO: 062, an amino acid sequence comprising SEQ ID NO: 064, and an amino acid sequence comprising SEQ ID NO:066.

29-35. (canceled)

36. A nucleic acid molecule, wherein the nucleic acid molecule encodes the chimeric antigen receptor according to claim 1.

37. The nucleic acid molecule according to claim 36, wherein the nucleic acid molecule comprises at least one of an extracellular target molecule binding domain nucleic acid fragment, a transmembrane domain nucleic acid fragment, an intracellular activation signaling domain nucleic acid fragment, an extracellular spacer domain nucleic acid fragment, an intracellular detection signaling nucleic acid fragment, an intracellular spacer domain nucleic acid fragment, and an intracellular linker domain nucleic acid fragment.

38. The nucleic acid molecule of claim 37, wherein the extracellular target molecule binding domain nucleic acid fragment comprises at least one of a nucleic acid sequence comprising SEQ ID NO:_002, a nucleic acid sequence comprising SEQ ID NO:_004, a nucleic acid sequence comprising SEQ ID NO:_006, a nucleic acid sequence comprising SEQ ID NO: 008, and a nucleic acid sequence comprising SEQ ID NO: 010;
preferably, the transmembrane domain nucleic acid fragment comprises at least one of a nucleic acid sequence comprising SEQ ID NO: 013 and a nucleic acid sequence comprising SEQ ID NO:015;
preferably, the intracellular activation signaling domain nucleic acid fragment comprises at least one of a nucleic acid sequence comprising SEQ ID NO: 043, a nucleic acid sequence comprising SEQ ID NO: 045, a nucleic acid sequence comprising SEQ ID NO: 047, a nucleic acid sequence comprising SEQ ID NO: 049, a nucleic acid sequence comprising SEQ ID NO: 051, and a nucleic acid sequence comprising SEQ ID NO: 053;
preferably, the extracellular spacer domain nucleic acid fragment comprises at least one of a nucleic acid sequence comprising SEQ ID NO: 017 and a nucleic acid sequence comprising SEQ ID NO: 019;
preferably, the intracellular detection signaling domain nucleic acid fragment comprises at least one of a nucleic acid sequence comprising SEQ ID NO: 021, a nucleic acid sequence comprising SEQ ID NO: 023, a nucleic acid sequence comprising SEQ ID NO: 025, a nucleic acid sequence comprising SEQ ID NO: 027, a nucleic acid sequence comprising SEQ ID NO: 029, a nucleic acid sequence comprising SEQ ID NO: 031, a nucleic acid sequence comprising SEQ ID NO: 033, a nucleic acid sequence comprising SEQ ID NO: 035, a nucleic acid sequence comprising SEQ ID NO: 037, a nucleic acid sequence comprising SEQ ID NO: 039, and a nucleic acid sequence comprising SEQ ID NO:041;
preferably, the intracellular spacer domain nucleic acid fragment comprises at least one of a nucleic acid sequence comprising SEQ ID NO: 055 and a nucleic acid sequence comprising SEQ ID NO: 057;
preferably, the intracellular linker domain nucleic acid fragment comprises at least one of a nucleic acid sequence comprising SEQ ID NO: 059, a nucleic acid sequence comprising SEQ ID NO: 061, a nucleic acid sequence comprising SEQ ID NO: 063, and a nucleic acid sequence comprising SEQ ID NO: 065.

39-48. (canceled)

49. A pharmaceutical composition, comprising the chimeric antigen receptor according to claim 1.

50. The pharmaceutical composition according to claim 49, further comprising cytokine;
preferably, the cytokine is at least one of interferon-γ and interleukin.

51. (canceled)

52. The pharmaceutical composition according to claim 49, further comprising monoclonal antibody.

53. The pharmaceutical composition according to claim 52, wherein the monoclonal antibody is at least one of cetuximab, alemtuzumab, ipilimumab, and ofatumumab.

54-67. (canceled)


SEQUENCE LISTING (66 sequences)

SEQ ID NO: 1 - protein, 117 aa, Artificial Sequence
  The sequence is synthetized.
PPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAE

SEQ ID NO: 2 - DNA, 351 bp, Artificial Sequence
  The sequence is synthetized.
ccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaa

SEQ ID NO: 3 - protein, 110 aa, Artificial Sequence
  The sequence is synthetized.
PTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRV

SEQ ID NO: 4 - DNA, 330 bp, Artificial Sequence
  The sequence is synthetized.
cccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtg

SEQ ID NO: 5 - protein, 121 aa, Artificial Sequence
  The sequence is synthetized.
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRV

SEQ ID NO: 6 - DNA, 363 bp, Artificial Sequence
  The sequence is synthetized.
ttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtg

SEQ ID NO: 7 - protein, 251 aa, Artificial Sequence
  The sequence is synthetized.
QVQLQQSGPGLVKPSQTLSLTCAISGDSVSSTKAAWYWIRQSPSRGLEWLGRTYFRSKWYNDYADSVKSRLTINPDTSKNQFSLQLKSVSPEDTAVYYCARGQYTAFDIWGQGTMVTVSSGILGSGGGGSGGGGSGGGGSQSALIQPASVSGSPGQSITISCTGTSSDVGGYDLVSWYQQYPGQAPRLIIYEVIKRPSGISDRFSGSKSGNTASLTISGLQAEDEADYYCCSYAGRRLHGVFGGGTQLTVL

SEQ ID NO: 8 - DNA, 753 bp, Artificial Sequence
  The sequence is synthetized.
caggtacagctgcagcagtcaggtccaggactggtgaagccctcgcagaccctctcactcacctgtgccatctccggggacagtgtctctagcaccaaggctgcttggtactggatcaggcagtccccttcgagaggccttgagtggctgggaaggacatacttccggtccaagtggtataatgactatgccgactctgtgaaaagtcgattaaccatcaacccagacacatccaagaaccagttctccctgcaacttaagtctgtgagtcccgaggacacggctgtgtattactgtgcaagagggcaatacactgcttttgatatctggggccaagggacaatggtcaccgtctcttcaggaattctaggatccggtggcggtggcagcggcggtggtggttccggaggcggcggttctcagtctgctctgattcagcctgcctccgtgtctgggtcccctggacagtcgatcactatctcctgtactggcaccagtagtgatgttggaggttatgaccttgtctcctggtaccaacagtacccgggccaagcccccagactcatcatttatgaggtcattaagcggccctcagggatttctgatcgcttctctggttccaagtctggcaacacggcctccctgacaatctctgggctccaggctgaggacgaggctgattattattgctgctcatatgcaggtagacgtcttcatggtgtgttcggaggaggcacccagctgaccgtcctc

SEQ ID NO: 9 - protein, 245 aa, Artificial Sequence
  The sequence is synthetized.
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGGGGSGGGGSGGGGSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 10 - DNA, 735 bp, Artificial Sequence
  The sequence is synthetized.
cagtccgccctgacccagcctgcctccgtgtctggctcccctggccagtccatcaccatcagctgcaccggcacctccagcgacgtgggcggctacaactacgtgtcctggtatcagcagcaccccggcaaggcccccaagctgatgatctacgacgtgtccaaccggccctccggcgtgtccaacagattctccggctccaagtccggcaacaccgcctccctgaccatcagcggactgcaggcagaggacgaggccgactactactgctcctcctacacctcctccagcaccagagtgttcggcaccggcacaaaagtgaccgtgctgggagggggcggttccggaggaggcggcagcgggggaggaggtagcgaggtgcagctgctggaatccggcggaggactggtgcagcctggcggctccctgagactgtcttgcgccgcctccggcttcaccttctccagctacatcatgatgtgggtgcgacaggcccctggcaagggcctggaatgggtgtcctccatctacccctccggcggcatcaccttctacgccgacaccgtgaagggccggttcaccatctcccgggacaactccaagaacaccctgtacctgcagatgaactccctgcgggccgaggacaccgccgtgtactactgcgcccggatcaagctgggcaccgtgaccaccgtggactactggggccagggcaccctggtgacagtgtcctcc

SEQ ID NO: 11 - protein, 254 aa, Artificial Sequence
  The sequence is synthetized.
EIVMTQSPSTLSASVGDRVIITCQASEDIYSLLAWYQQKPGKAPKLLIYDASDLASGVPSRFSGSGSGAEFTLTISSLQPDDFATYYCQGNYGSSSSSSYGAVFGQGTKLTVLGGGGGSGGGGSGGGGSGGGGSEVQLVESGGGLVQPGGSLRLSCTVSGIDLSSYTMGWVRQAPGKGLEWVGIISSGGRTYYASWAKGRFTISRDTSKNTVYLQMNSLRAEDTAVYYCARGRYTGYPYYFALWGQGTLVTVSS

SEQ ID NO: 12 - protein, 21 aa, Artificial Sequence
  The sequence is synthetized.
VGVVGGLLGSLVLLVWVLAVI

SEQ ID NO: 13 - DNA, 63 bp, Artificial Sequence
  The sequence is synthetized.
gttggtgtcgtgggcggcctgctgggcagcctggtgctgctagtctgggtcctggccgtcatc

SEQ ID NO: 14 - protein, 24 aa, Artificial Sequence
  The sequence is synthetized.
TLVVGVVGGLLGSLVLLVWVLAVI

SEQ ID NO: 15 - DNA, 72 bp, Artificial Sequence
  The sequence is synthetized.
accctggtggttggtgtcgtgggcggcctgctgggcagcctggtgctgctagtctgggtcctggccgtcatc

SEQ ID NO: 16 - protein, 20 aa, Artificial Sequence
  The sequence is synthetized.
VPTAHPSPSPRPAGQFQTLV

SEQ ID NO: 17 - DNA, 60 bp, Artificial Sequence
  The sequence is synthetized.
gtgcccacagcccaccccagcccctcacccaggccagccggccagttccaaaccctggtg

SEQ ID NO: 18 - protein, 23 aa, Artificial Sequence
  The sequence is synthetized.
TERRAEVPTAHPSPSPRPAGQFQ

SEQ ID NO: 19 - DNA, 69 bp, Artificial Sequence
  The sequence is synthetized.
acagagagaagggcagaagtgcccacagcccaccccagcccctcacccaggccagccggccagttccaa

SEQ ID NO: 20 - protein, 20 aa, Artificial Sequence
  The sequence is synthetized.
NQLYNELNLGRREEYDVLDK

SEQ ID NO: 21 - DNA, 60 bp, Artificial Sequence
  The sequence is synthetized.
aaccagctctataacgagctcaatctaggacgaagagaggagtacgatgttttggacaag

SEQ ID NO: 22 - protein, 20 aa, Artificial Sequence
  The sequence is synthetized.
NQLFNELNLGRREEFDVLDK

SEQ ID NO: 23 - DNA, 60 bp, Artificial Sequence
  The sequence is synthetized.
aaccagctctttaacgagctcaatctaggacgaagagaggagttcgatgttttggacaag

SEQ ID NO: 24 - protein, 21 aa, Artificial Sequence
  The sequence is synthetized.
EGLYNELQKDKMAEAYSEIGM

SEQ ID NO: 25 - DNA, 63 bp, Artificial Sequence
  The sequence is synthetized.
gaaggcctgtacaatgaactgcagaaagataagatggcggaggcctacagtgagattgggatg

SEQ ID NO: 26 - protein, 21 aa, Artificial Sequence
  The sequence is synthetized.
EGLFNELQKDKMAEAFSEIGM

SEQ ID NO: 27 - DNA, 63 bp, Artificial Sequence
  The sequence is synthetized.
gaaggcctgttcaatgaactgcagaaagataagatggcggaggccttcagtgagattgggatg

SEQ ID NO: 28 - protein, 20 aa, Artificial Sequence
  The sequence is synthetized.
DGLYQGLSTATKDTYDALHM

SEQ ID NO: 29 - DNA, 60 bp, Artificial Sequence
  The sequence is synthetized.
gatggcctttaccagggactcagtacagccaccaaggacacctacgacgcccttcacatg

SEQ ID NO: 30 - protein, 20 aa, Artificial Sequence
  The sequence is synthetized.
DGLFQGLSTATKDTFDALHM

SEQ ID NO: 31 - DNA, 60 bp, Artificial Sequence
  The sequence is synthetized.
gatggccttttccagggactcagtacagccaccaaggacaccttcgacgcccttcacatg

SEQ ID NO: 32 - protein, 20 aa, Artificial Sequence
  The sequence is synthetized.
PDYEPIRKGQRDLYSGLNQR

SEQ ID NO: 33 - DNA, 60 bp, Artificial Sequence
  The sequence is synthetized.
ccagactatgagcccatccggaaaggccagcgggacctgtattctggcctgaatcagaga

SEQ ID NO: 34 - protein, 20 aa, Artificial Sequence
  The sequence is synthetized.
PDFEPIRKGQRDLFSGLNQR

SEQ ID NO: 35 - DNA, 60 bp, Artificial Sequence
  The sequence is synthetized.
ccagactttgagcccatccggaaaggccagcgggacctgttttctggcctgaatcagaga

SEQ ID NO: 36 - protein, 27 aa, Artificial Sequence
  The sequence is synthetized.
GGYMTLNPRAPTDDDKNIYLTLPPNGT

SEQ ID NO: 37 - DNA, 81 bp, Artificial Sequence
  The sequence is synthetized.
ggcggctacatgactctgaaccccagggcacctactgacgatgataaaaacatctacctgactcttcctcccaacggtacc

SEQ ID NO: 38 - protein, 20 aa, Artificial Sequence
  The sequence is synthetized.
GVYTGLSTRNQETYETLKHE

SEQ ID NO: 39 - DNA, 60 bp, Artificial Sequence
  The sequence is synthetized.
ggtgtttacacgggcctgagcaccaggaaccaggagacttacgagactctgaagcatgag

SEQ ID NO: 40 - protein, 22 aa, Artificial Sequence
  The sequence is synthetized.
SPYQELQGQRSDVYSDLNTQGT

SEQ ID NO: 41 - DNA, 66 bp, Artificial Sequence
  The sequence is synthetized.
tcgccttatcaggaactccagggtcagaggtcggatgtctacagcgacctcaacacacagggtacc

SEQ ID NO: 42 - protein, 618 aa, Artificial Sequence
  The sequence is synthetized.
PDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 43 - DNA, 1854 bp, Artificial Sequence
  The sequence is synthetized.
ccagaccccgcggcgcacctgcccttcttctacggcagcatctcgcgtgccgaggccgaggagcacctgaagctggcgggcatggcggacgggctcttcctgctgcgccagtgcctgcgctcgctgggcggctatgtgctgtcgctcgtgcacgatgtgcgcttccaccactttcccatcgagcgccagctcaacggcacctacgccattgccggcggcaaagcgcactgtggaccggcagagctctgcgagttctactcgcgcgaccccgacgggctgccctgcaacctgcgcaagccgtgcaaccggccgtcgggcctcgagccgcagccgggggtcttcgactgcctgcgagacgccatggtgcgtgactacgtgcgccagacgtggaagctggagggcgaggccctggagcaggccatcatcagccaggccccgcaggtggagaagctcattgctacgacggcccacgagcggatgccctggtaccacagcagcctgacgcgtgaggaggccgagcgcaaactttactctggggcgcagaccgacggcaagttcctgctgaggccgcggaaggagcagggcacatacgccctgtccctcatctatgggaagacggtgtaccactacctcatcagccaagacaaggcgggcaagtactgcattcccgagggcaccaagtttgacacgctctggcagctggtggagtatctgaagctgaaggcggacgggctcatctactgcctgaaggaggcctgccccaacagcagtgccagcaacgcctcaggggctgctgctcccacactcccagcccacccatccacgttgactcatcctcagagacgaatcgacaccctcaactcagatggatacacccctgagccagcacgcataacgtccccagacaaaccgcggccgatgcccatggacacgagcgtgtatgagagcccctacagcgacccagaggagctcaaggacaagaagctcttcctgaagcgcgataacctcctcatagctgacattgaacttggctgcggcaactttggctcagtgcgccagggcgtgtaccgcatgcgcaagaagcagatcgacgtggccatcaaggtgctgaagcagggcacggagaaggcagacacggaagagatgatgcgcgaggcgcagatcatgcaccagctggacaacccctacatcgtgcggctcattggcgtctgccaggccgaggccctcatgctggtcatggagatggctgggggcgggccgctgcacaagttcctggtcggcaagagggaggagatccctgtgagcaatgtggccgagctgctgcaccaggtgtccatggggatgaagtacctggaggagaagaactttgtgcaccgtgacctggcggcccgcaacgtcctgctggttaaccggcactacgccaagatcagcgactttggcctctccaaagcactgggtgccgacgacagctactacactgcccgctcagcagggaagtggccgctcaagtggtacgcacccgaatgcatcaacttccgcaagttctccagccgcagcgatgtctggagctatggggtcaccatgtgggaggccttgtcctacggccagaagccctacaagaagatgaaagggccggaggtcatggccttcatcgagcagggcaagcggatggagtgcccaccagagtgtccacccgaactgtacgcactcatgagtgactgctggatctacaagtgggaggatcgccccgacttcctgaccgtggagcagcgcatgcgagcctgttactacagcctggccagcaaggtggaagggcccccaggcagcacacagaaggctgaggctgcctgtgcc

SEQ ID NO: 44 - protein, 634 aa, Artificial Sequence
  The sequence is synthetized.
ASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKIGTQGNVNFGGRPQLPGSHPATWSAGGIISRIKSYSFPKPGHRKSSPAQGNRQESTVSFNPYEPELAPWAADKGPQREALPMDTEVYESPYADPEEIRPKEVYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYYDVVN

SEQ ID NO: 45 - DNA, 1902 bp, Artificial Sequence
  The sequence is synthetized.
gccagcagcggcatggctgacagcgccaaccacctgcccttctttttcggcaacatcacccgggaggaggcagaagattacctggtccaggggggcatgagtgatgggctttatttgctgcgccagagccgcaactacctgggtggcttcgccctgtccgtggcccacgggaggaaggcacaccactacaccatcgagcgggagctgaatggcacctacgccatcgccggtggcaggacccatgccagccccgccgacctctgccactaccactcccaggagtctgatggcctggtctgcctcctcaagaagcccttcaaccggccccaaggggtgcagcccaagactgggccctttgaggatttgaaggaaaacctcatcagggaatatgtgaagcagacatggaacctgcagggtcaggctctggagcaggccatcatcagtcagaagcctcagctggagaagctgatcgctaccacagcccatgaaaaaatgccttggttccatggaaaaatctctcgggaagaatctgagcaaattgtcctgataggatcaaagacaaatggaaagttcctgatccgagccagagacaacaacggctcctacgccctgtgcctgctgcacgaagggaaggtgctgcactatcgcatcgacaaagacaagacagggaagctctccatccccgagggaaagaagttcgacacgctctggcagctagtcgagcattattcttataaagcagatggtttgttaagagttcttactgtcccatgtcaaaaaatcggcacacagggaaatgttaattttggaggccgtccacaacttccaggttcccatcctgcgacttggtcagcgggtggaataatctcaagaatcaaatcatactccttcccaaagcctggccacagaaagtcctcccctgcccaagggaaccggcaagagagtactgtgtcattcaatccgtatgagccagaacttgcaccctgggctgcagacaaaggcccccagagagaagccctacccatggacacagaggtgtacgagagcccctacgcggaccccgaggagatcaggcccaaggaggtttacctggaccgaaagctgctgacgctggaagacaaagaactgggctctggtaattttggaactgtgaaaaagggctactaccaaatgaaaaaagttgtgaaaaccgtggctgtgaaaatactgaaaaacgaggccaatgaccccgctcttaaagatgagttattagcagaagcaaatgtcatgcagcagctggacaacccgtacatcgtgcggatgatcgggatatgcgaggccgagtcctggatgctggttatggagatggcagaacttggtcccctcaataagtatttgcagcagaacagacatgtcaaggataagaacatcatagaactggttcatcaggtttccatgggcatgaagtacttggaggagagcaattttgtgcacagagatctggctgcaagaaatgtgttgctagttacccaacattacgccaagatcagtgatttcggactttccaaagcactgcgtgctgatgaaaactactacaaggcccagacccatggaaagtggcctgtcaagtggtacgctccggaatgcatcaactactacaagttctccagcaaaagcgatgtctggagctttggagtgttgatgtgggaagcattctcctatgggcagaagccatatcgagggatgaaaggaagtgaagtcaccgctatgttagagaaaggagagcggatggggtgccctgcagggtgtccaagagagatgtacgatctcatgaatctgtgctggacatacgatgtggaaaacaggcccggattcgcagcagtggaactgcggctgcgcaattactactatgacgtggtgaac

SEQ ID NO: 46 - protein, 336 aa, Artificial Sequence
  The sequence is synthetized.
PDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNL

SEQ ID NO: 47 - DNA, 1008 bp, Artificial Sequence
  The sequence is synthetized.
ccagaccccgcggcgcacctgcccttcttctacggcagcatctcgcgtgccgaggccgaggagcacctgaagctggcgggcatggcggacgggctcttcctgctgcgccagtgcctgcgctcgctgggcggctatgtgctgtcgctcgtgcacgatgtgcgcttccaccactttcccatcgagcgccagctcaacggcacctacgccattgccggcggcaaagcgcactgtggaccggcagagctctgcgagttctactcgcgcgaccccgacgggctgccctgcaacctgcgcaagccgtgcaaccggccgtcgggcctcgagccgcagccgggggtcttcgactgcctgcgagacgccatggtgcgtgactacgtgcgccagacgtggaagctggagggcgaggccctggagcaggccatcatcagccaggccccgcaggtggagaagctcattgctacgacggcccacgagcggatgccctggtaccacagcagcctgacgcgtgaggaggccgagcgcaaactttactctggggcgcagaccgacggcaagttcctgctgaggccgcggaaggagcagggcacatacgccctgtccctcatctatgggaagacggtgtaccactacctcatcagccaagacaaggcgggcaagtactgcattcccgagggcaccaagtttgacacgctctggcagctggtggagtatctgaagctgaaggcggacgggctcatctactgcctgaaggaggcctgccccaacagcagtgccagcaacgcctcaggggctgctgctcccacactcccagcccacccatccacgttgactcatcctcagagacgaatcgacaccctcaactcagatggatacacccctgagccagcacgcataacgtccccagacaaaccgcggccgatgcccatggacacgagcgtgtatgagagcccctacagcgacccagaggagctcaaggacaagaagctcttcctgaagcgcgataacctc

SEQ ID NO: 48 - protein, 369 aa, Artificial Sequence
  The sequence is synthetized.
ASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKIGTQGNVNFGGRPQLPGSHPATWSAGGIISRIKSYSFPKPGHRKSSPAQGNRQESTVSFNPYEPELAPWAADKGPQREALPMDTEVYESPYADPEEIRPKEVYLDRKLL

SEQ ID NO: 49 - DNA, 1107 bp, Artificial Sequence
  The sequence is synthetized.
gccagcagcggcatggctgacagcgccaaccacctgcccttctttttcggcaacatcacccgggaggaggcagaagattacctggtccaggggggcatgagtgatgggctttatttgctgcgccagagccgcaactacctgggtggcttcgccctgtccgtggcccacgggaggaaggcacaccactacaccatcgagcgggagctgaatggcacctacgccatcgccggtggcaggacccatgccagccccgccgacctctgccactaccactcccaggagtctgatggcctggtctgcctcctcaagaagcccttcaaccggccccaaggggtgcagcccaagactgggccctttgaggatttgaaggaaaacctcatcagggaatatgtgaagcagacatggaacctgcagggtcaggctctggagcaggccatcatcagtcagaagcctcagctggagaagctgatcgctaccacagcccatgaaaaaatgccttggttccatggaaaaatctctcgggaagaatctgagcaaattgtcctgataggatcaaagacaaatggaaagttcctgatccgagccagagacaacaacggctcctacgccctgtgcctgctgcacgaagggaaggtgctgcactatcgcatcgacaaagacaagacagggaagctctccatccccgagggaaagaagttcgacacgctctggcagctagtcgagcattattcttataaagcagatggtttgttaagagttcttactgtcccatgtcaaaaaatcggcacacagggaaatgttaattttggaggccgtccacaacttccaggttcccatcctgcgacttggtcagcgggtggaataatctcaagaatcaaatcatactccttcccaaagcctggccacagaaagtcctcccctgcccaagggaaccggcaagagagtactgtgtcattcaatccgtatgagccagaacttgcaccctgggctgcagacaaaggcccccagagagaagccctacccatggacacagaggtgtacgagagcccctacgcggaccccgaggagatcaggcccaaggaggtttacctggaccgaaagctgctg

SEQ ID NO: 50 - protein, 263 aa, Artificial Sequence
  The sequence is synthetized.
LIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSL

SEQ ID NO: 51 - DNA, 789 bp, Artificial Sequence
  The sequence is synthetized.
ctcatagctgacattgaacttggctgcggcaactttggctcagtgcgccagggcgtgtaccgcatgcgcaagaagcagatcgacgtggccatcaaggtgctgaagcagggcacggagaaggcagacacggaagagatgatgcgcgaggcgcagatcatgcaccagctggacaacccctacatcgtgcggctcattggcgtctgccaggccgaggccctcatgctggtcatggagatggctgggggcgggccgctgcacaagttcctggtcggcaagagggaggagatccctgtgagcaatgtggccgagctgctgcaccaggtgtccatggggatgaagtacctggaggagaagaactttgtgcaccgtgacctggcggcccgcaacgtcctgctggttaaccggcactacgccaagatcagcgactttggcctctccaaagcactgggtgccgacgacagctactacactgcccgctcagcagggaagtggccgctcaagtggtacgcacccgaatgcatcaacttccgcaagttctccagccgcagcgatgtctggagctatggggtcaccatgtgggaggccttgtcctacggccagaagccctacaagaagatgaaagggccggaggtcatggccttcatcgagcagggcaagcggatggagtgcccaccagagtgtccacccgaactgtacgcactcatgagtgactgctggatctacaagtgggaggatcgccccgacttcctgaccgtggagcagcgcatgcgagcctgttactacagcctg

SEQ ID NO: 52 - protein, 261 aa, Artificial Sequence
  The sequence is synthetized.
TLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYY

SEQ ID NO: 53 - DNA, 783 bp, Artificial Sequence
  The sequence is synthetized.
acgctggaagacaaagaactgggctctggtaattttggaactgtgaaaaagggctactaccaaatgaaaaaagttgtgaaaaccgtggctgtgaaaatactgaaaaacgaggccaatgaccccgctcttaaagatgagttattagcagaagcaaatgtcatgcagcagctggacaacccgtacatcgtgcggatgatcgggatatgcgaggccgagtcctggatgctggttatggagatggcagaacttggtcccctcaataagtatttgcagcagaacagacatgtcaaggataagaacatcatagaactggttcatcaggtttccatgggcatgaagtacttggaggagagcaattttgtgcacagagatctggctgcaagaaatgtgttgctagttacccaacattacgccaagatcagtgatttcggactttccaaagcactgcgtgctgatgaaaactactacaaggcccagacccatggaaagtggcctgtcaagtggtacgctccggaatgcatcaactactacaagttctccagcaaaagcgatgtctggagctttggagtgttgatgtgggaagcattctcctatgggcagaagccatatcgagggatgaaaggaagtgaagtcaccgctatgttagagaaaggagagcggatggggtgccctgcagggtgtccaagagagatgtacgatctcatgaatctgtgctggacatacgatgtggaaaacaggcccggattcgcagcagtggaactgcggctgcgcaattactactat

SEQ ID NO: 54 - protein, 29 aa, Artificial Sequence
  The sequence is synthetized.
CSRAARGTIGARRTGQPLKEDPSAVPVFS

SEQ ID NO: 55 - DNA, 87 bp, Artificial Sequence
  The sequence is synthetized.
tgctcccgggccgcacgagggacaataggagccaggcgcaccggccagcccctgaaggaggacccctcagccgtgcctgtgttctct

SEQ ID NO: 56 - protein, 97 aa, Artificial Sequence
  The sequence is synthetized.
CSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 57 - DNA, 291 bp, Artificial Sequence
  The sequence is synthetized.
tgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctc

SEQ ID NO: 58 - protein, 12 aa, Artificial Sequence
  The sequence is synthetized.
GGSGGTGGSGGT

SEQ ID NO: 59 - DNA, 36 bp, Artificial Sequence
  The sequence is synthetized.
ggcgggtctggcgggacaggaggttcaggtggcaca

SEQ ID NO: 60 - protein, 34 aa, Artificial Sequence
  The sequence is synthetized.
GSTSGSGKPGSGEGSTKGSTSGSGKPGSGEGSTK

SEQ ID NO: 61 - DNA, 102 bp, Artificial Sequence
  The sequence is synthetized.
ggttcaacttctggctctgggaaaccaggaagcggcgaagggtccaccaagggaagcaccagtggttcaggtaagcctggttctggtgaaggtagcactaaa

SEQ ID NO: 62 - protein, 128 aa, Artificial Sequence
  The sequence is synthetized.
SAGGSAGGSAGGSAGGSAGGSGSAGGSAGGSTSAGGSAGGSAGGSAGGSAGGSGSAGGSAGGSTSAGGSAGGSAGGSAGGSAGGSGSAGGSAGGSTSAGGSAGGSAGGSAGGSAGGGGSGGTGGSGGT

SEQ ID NO: 63 - DNA, 384 bp, Artificial Sequence
  The sequence is synthetized.
agcgcaggcggatcagctggagggtctgcagggggtagtgcaggtggctcagctggcgggagcggctcagctgggggatctgctggtggcagtacctcagcaggcggtagcgccggaggttctgctggtggctccgcaggagggtctgcaggcggttccgggagtgcaggtggatctgcaggtgggtcaacaagtgctggtggatccgcaggaggttcagcaggcgggagtgctggaggctctgcaggcggtagcgggagtgccggtggcagcgcagggggaagcactagtgctggaggcagtgcaggtggcagcgcaggaggctctgccgggggaagcgccgggggcggcgggtctggcgggacaggaggttcaggtggcaca

SEQ ID NO: 64 - protein, 116 aa, Artificial Sequence
  The sequence is synthetized.
SAGGSAGGSAGGSAGGSAGGSGSAGGSAGGSTSAGGSAGGSAGGSAGGSAGGSGSAGGSAGGSTSAGGSAGGSAGGSAGGSAGGSGSAGGSAGGSTSAGGSAGGSAGGSAGGSAGG

SEQ ID NO: 65 - DNA, 348 bp, Artificial Sequence
  The sequence is synthetized.
agcgcaggcggatcagctggagggtctgcagggggtagtgcaggtggctcagctggcgggagcggctcagctgggggatctgctggtggcagtacctcagcaggcggtagcgccggaggttctgctggtggctccgcaggagggtctgcaggcggttccgggagtgcaggtggatctgcaggtgggtcaacaagtgctggtggatccgcaggaggttcagcaggcgggagtgctggaggctctgcaggcggtagcgggagtgccggtggcagcgcagggggaagcactagtgctggaggcagtgcaggtggcagcgcaggaggctctgccgggggaagcgccgggggc

SEQ ID NO: 66 - protein, 6 aa, Artificial Sequence
  The sequence is synthetized.
GGSGGT